SEQUENCE LISTING (47 sequences)

SEQ ID NO: 1 - DNA, 1983 bp, Escherichia coli
  source 1..1983: mol_type = genomic DNA
atgtcacgtccgcaatttacctcgttgcgtttgagtttgttggctttggctgtttctgccaccttgccaacgtttgcttttgctactgaaaccatgaccgttacggcaacggggaatgcacgtagttccttcgaagcgcctatgatggtcagcgttatcgacacttccgctcctgaaaatcaaactgctacttcagccactgatttgctgcgtcatgttcctggaattactcttgatggtaccggacgaaccaacggtcaggatgtaaatatgcgtggctatgatcatcgcggcgtgctggttcttgtcgatggtgttcgccagggaacggataccggacacctgaatggcacttttctcgatccggcgctgatcaagcgtgttgagattgttcgcggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgaatccattaataacatgctggcgaaagggacctggcaaattgattcagcccagtctctgagcggtttagtgcgttactacaacaacgacgcgcgtgaaccaaaaaatccgcagaccgttgaagcttctgatagcagtaacccgatggtcgatcgttcaacaattcaacgcgatgcgcagctttcttataaactcgccccgcagggtaacgactggttaaatgcagatgcaaaaatttactggtcggaagtccgtattaatgcgcaaaacacggggagttccggcgagtatcgtgaacagataacaaaaggagcaaggctggagaaccgttccactctatttgccgacagtttcgcttctcacttactgacatatggcggtgagtattatcgtcaggaacaacatccgggtggcgcgacgacgggcttcccgcaagcaaaaatcgattttagctctggctggctacaagatgagatcaccttacgcgatctgccgattaccctgcttggcggaacccgctatgacagttatcgcggtagcagcgacggctacaaagatgttgatgccgacaaatggtcatctcgtgcggggatgactatcaacccgaccaactggctgatgttatttggctcatatgctcaggcattccgcgccccgacgatgggcgaaatgtataacgattctaaacacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaagattatatctccacgaccgtcgatttcgcggcggcgacaactatgtcgtataacgtcccgaacgccaaaatctggggctgggatgtgatgacgaaatataccactgatctgtttagccttgatgtggcctataaccgtacccgcggcaaagacaccgataccggggaatatatctccagcattaacccggataccgttaccagtaccctgaatattccgatcgctcacagtggcttctctgttggttgggtcggtacgtttgccgatcgctcaacacatatcagcagcagctacagcaaacaacctggctatggtgtgaatgatttctacgtcagttatcaagggcagcaggcgctcaaaggcatgaccactactctggtattgggcaacgccttcgataaagagtactggtcgccgcaaggcatcccacaggatggtcgtaacggaaaaattttcgtgagttatcaatggtaa

SEQ ID NO: 2 - protein, 632 aa, Escherichia coli
TETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 3 - DNA, 2049 bp, Escherichia coli
  source 1..2049: mol_type = genomic DNA
atgaagaacaaatatatcattgctccgggcattgccgtgatgtgttctgcagttatatcatcaggttatgccagttctgataaaaaagaagatacgcttgttgttactgcctccgggttcactcagcagctcagaaatgccccggccagtgtctcagtcattacttcagaacaactgcaaaaaaaaccggtttcagatctggtcgatgcagtaaaagatgttgaagggattagtatcactggtgggaatgaaaaaccggatatcagtatacgtggtctaagtggcgattacacgctgattctggtcgatggacgacgtcagagcggtcgggaatccagaccaaacggcagcggcggttttgaagccggatttatccctcctgtggaagcaattgaacgcattgaagtgatccgtggccctatgtcttccctgtatggttctgatgccatcggaggggtcattaatatcataaccaaaccagttaataaccaaacatgggatggcgtacttggacttggggggattattcaggaacatgggaaatttggtaactcaaccacaaatgacttctatctgtcaggcccattgattaaggataaacttggtcttcagctatatggaggaatgaactatcgcaaggaagatagtatctctcagggaacaccggcaaaagataataagaatataacggcaacgctccagtttactccgactgaaagccagaagtttgtttttgaatatggaaaaaataaccaggtgcatacattaacacctggtgagtctctcgatgcctggactatgcggggaaatcttaaacaaccaaacagtaaaagagaaacgcataattcacgtagtcactgggtagcagcatggaatgcccagggcgaaatactgcatcctgaaattgctgtttatcaggagaaagttattcgtgagattaaatcaggtaaaaaagataaatataatcattgggatcttaattacgagtcaagaaaaccggaaataaccaacacaatcatagatgcaaaagtgacggcatttctgccggaaaatgtactgaccatcggaggtcaatttcagcatgcagagctccgtgatgactcagccacgggtaaaaaaacgacagaaacacagtctgtttcaattaaacagaaagctgtttttatagaaaatgaatatgcagcaacggattctctcgccctgactggaggactgcgtctcgataatcatgaaatctatggcagttactggaatccaagattgtacgctgtttataacctgaccgataatctcacactcaaaggggggatcgcaaaagcatttcgggctccttcaattcgtgaggtgagtcctggatttggaacactgacgcagggtggtgcctctattatgtatggaaacagggacctgaaaccggagaccagtgtaaccgaagagatcggtattatttatagtaatgatagtggtttttcggcgagcgcgacgctgtttaatactgattttaaaaataagttgaccagttacgatataggtacaaaagatccagtcaccgggttaaacacttttatttatgataatgtaggtgaggcaaatatcagaggggtggagcttgcaactcagattcctgtgtatgataaatggcatgtatctgcaaactatacatttactgactctcgtcgaaaaagtgatgacgaaagtctcaatggcaagtcgctgaaaggggaacctctggaaagaactcccagacatgcagccaatgcaaaactggaatgggattacactcaggatattacattttattcatctctgaattatacgggaaaacaaatctgggcagcacaaagaaatggtgctaaggttccccgcgttcgtaatggattcacatctatggatattggtctaaattaccagattctgccagacacgctgattaattttgccgttcttaacgtcacagacagaaagagcgaggatatcgataccattgatggtaactggcaggtcgatgaaggacgccgttattgggctaatgtaagagtatccttctga

SEQ ID NO: 4 - protein, 658 aa, Escherichia coli
SSDKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREIKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 5 - DNA, 2178 bp, Escherichia coli
  source 1..2178: mol_type = genomic DNA
atgagaactcacaagctcctctggccgctaaccgcgctcctggttgggtttaatagccaggtatcagtagctaaatcctccgacgacgataatgacgagactctggtggtggaagccaccgctgagcagatattaaaacagcagccgggagtgtcggttattaccagcgaggatattaaaaagtcccctccggtaaacgacatttcagatattattcgtaaaatgcctggtgtcaatcttaccggcaatagcgcctcgggcacacgcggtaataaccgccaaatcgatattcgcggtatggggccggaaaacaccttaattttaattgatggtgtaccggtgacgtcacgtaactccgtgcgttatagctggcgtggagagcgtgatacccgcggtgacaccaactgggtgccaccggaacaggttgagcgtattgaagtgatccgcggccctgcggcggcgcgctacggttcaggggcagccgggggggtggtgaacatcattaccaaacgtcccaccaacgactggcacggttcgctgtcgttatacaccaaccagccggaaagtagcgatgagggcgctacgcgtcgcgccaatttcagccttagtgggcctctggctggtaatgctcttaccacgcgtttgtacggtaacctgaataaaacggatgccgacagctgggatattaactcctcggccggtacgaaaaacgcagccgggcatgaaggggtacgtaacaaagatattaacggcgttgtctcgtggaaattaaatccgcagcagattctcgatttcgaagccggatatagccgccaggggaatatctatgcgggcgatacgcaaaacagttcttccagtgcagttaccgaaagcctggcaaaatccggcaaagagacgaaccgcctgtaccgacagaattatggcattacgcataatggtatctgggactggggacaaagtcgctttggtgtttattacgagaaaaccaataatacccgcatgaatgaaggattatccggcggtggtgaagggcgtattttagcgggtgaaaagtttacgaccaatcgcctgagttcctggcgaaccagcggcgagcttaatattcctttgaatgtgatggttgatcaaacgctgaccgttggtgcagagtggaaccgcgataagctcgatgatccttcctctaccagcctgacggtgaatgacagatatatcagcggtatttctggctctgctgcggatcgcagcagtaaaaatcattctcaaatcagtgcgctgtatattgaagataacattgagccggttcctggcacgaatatcattcccggcctgcgctttgattatctcagcgactccggcgggaacttcagccccagtctgaatctttcgcaggaattgggcgattatttcaaagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattgggctggagttcacctgggaagattaccacgcaagtgtgacctacttccgcaatgattaccagaataagatcgtggccggggataacgttatcgggcaaaccgcttcaggcgcatatatcctcaagtggcagaatggcggaaaagctctggtggacggtatcgaagccagtatgtctttcccactggtgaaagatcgtctgaactggaataccaatgccacatggatgatcacttcggagcaaaaagacaccggtaatcctctgtcggtcatcccgaaatatactatcaataactcgcttaactggaccatcacccaggcgttttctgccagcgtcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtcagtaatatcctcaataaacagatcttccgatcttctgaaggggcgaatacctataacgagccaggccgggcttactatgccggagttaccgcatcattctga

SEQ ID NO: 6 - protein, 701 aa, Escherichia coli
KSSDDDNDETLVVEATAEQILKQQPGVSVITSEDIKKSPPVNDISDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSDEGATRRANFSLSGPLAGNALTTRLYGNLNKTDADSWDINSSAGTKNAAGHEGVRNKDINGVVSWKLNPQQILDFEAGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRYISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKDRLNWNTNATWMITSEQKDTGNPLSVIPKYTINNSLNWTITQAFSASVNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 7 - DNA, 2241 bp, Escherichia coli
  source 1..2241: mol_type = genomic DNA
atgaacaagaagattcattccctggccttgttggtcaatctggggatttatggggtagcacaggcacaagagccgaccgatactcctgtttcacatgacgatactattgttgttaccgccgccgagcagaacttgcaggcgcccggcgtttcgaccatcaccgcagatgaaatccgcaaaaacccggttgcccgcgatgtatcggagattatccgtaccatgcctggcgttaacctgaccggtaattccaccagtggtcagcgtggtaataaccgccagattgatattcgcggcatgggtccggaaaacacgctgattttgattgacggcaagccagtaagcagccgtaactcagtgcgtcagggctggcgtggcgagcgcgatacccgtggtgatacctcctgggtgccgcctgaaatgattgaacgtattgaagttctgcgtggtccggcagctgcgcgttatggcaacggcgcggcgggcggcgtggttaacatcattaccaaaaaaggcagcggtgagtggcacggctcctgggatgcttatttcaatgcgccagaacataaagaggaaggtgccaccaaacgcaccaacttcagcctgaccggtccgctgggcgacgaattcagcttccgcttgtatggcaacctcgacaaaacccaggctgacgcgtgggatatcaaccagggccatcagtccgcgcgtgccggaacgtatgccacgacgttaccagccgggcgcgaaggggtgatcaacaaagatattaatggcgtggtgcgctgggacttcgcgcctctgcagtcactcgaactggaagcgggctacagccgccagggtaacctgtatgcgggtgatacgcagaacaccaactctgacgcttacactcgatcgaaatatggcgatgaaaccaaccgcctgtatcgccagaactactcgctgacctggaacggtggctgggataacggcgtgaccaccagcaactgggtgcagtacgaacacacccgtaactcgcgtattccggaaggtctggcgggcggtaccgaagggaaatttaacgaaaaagcggcacaggattttgtagatatcgatcttgatgacgtgatgctgcacagcgaagttaacctgccgattgatttcctcgtaaaccaaacgctgacgctgggtacagagtggaatcagcaacggatgaaggacttaagttccaacacccaggcgctgaccggaacgaataccggcggtgctattgatggtgtgagtgccaccgaccgtagcccgtattcaaaagcagaaattttctcgctgtttgccgaaaacaatatggagctgactgacagcaccatcgtaacgccggggctgcgtttcgatcatcacagtattgtcggcaataactggagcccggcgctgaacatatcgcaaggtttaggcgatgacttcacgctgaaaatgggcatcgcccgcgcctataaagcgccgagtctgtaccagactaacccaaactacattctctacagtaaaggtcagggctgctatgccagcgcgggcggctgctatctgcaaggtaatgatgacctgaaagcagaaaccagcatcaacaaggagattggtctggagttcaaacgcgacggctggctggcgggcgtgacctggttccgtaacgattatcgcaataagattgaagcgggctatgtggctgtagggcaaaacgcagtcggcaccgatctctatcagtgggataacgtaccgaaagcggtggttgaaggtctggaaggatcgttaaacgtaccggttagcgaaacggtgatgtggaccaataacatcacttatatgctgaagagtgaaaacaaaaccacgggcgaccgtttgtcgatcatcccggaatatacgttgaactcaacgctgagctggcaggcacgggaagatttgtcgatgcaaacgaccttcacctggtacggcaaacagcagccgaagaagtacaactataaaggtcagccagcggttggaccggaaaccaaagaaatcagtccgtacagcattgttggcctgagcgcgacctgggatgtgaccaagaatgtcagtctgaccggcggcgtggacaacctgttcgacaaacgtttgtggcgtgcgggtaatgcccagaccacgggcgatctggcaggagccaactatatcgcaggtgccggggcgtatacctataacgagccgggacgtacgtggtatatgagcattaatactcacttctga

SEQ ID NO: 8 - protein, 724 aa, Escherichia coli
QEPTDTPVSHDDTIVVTAAEQNLQAPGVSTITADEIRKNPVARDVSEIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDAYTRSKYGDETNRLYRQNYSLTWNGGWDNGVTTSNWVQYEHTRNSRIPEGLAGGTEGKFNEKAAQDFVDIDLDDVMLHSEVNLPIDFLVNQTLTLGTEWNQQRMKDLSSNTQALTGTNTGGAIDGVSATDRSPYSKAEIFSLFAENNMELTDSTIVTPGLRFDHHSIVGNNWSPALNISQGLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSINTHF

SEQ ID NO: 9 - DNA, 2325 bp, Escherichia coli
  source 1..2325: mol_type = genomic DNA
atgacgccgttacgcgtttttcgtaaaacaacacctttggttaacgccattcgcctgagcctgctgccgctggccggtctctcgttttccgcttttgctgcacaggttaatatcgcaccgggatcgctcgataaagcgctcaatcagtatgccgcacacagcggatttaccctctcggttgacgccagcctgacgcgcggcaagcagagcaacggcctgcacggcgattacgacgtcgagagcggcctgcaacaactgctggacggcagcggactgcaggtaaaaccgctgggaaataatagctggacgctggagcccgcgcccgcaccaaaagaagatgccctgaccgtggtcggcgactggctgggtgatgcgcgtgaaaacgacgtatttgaacatgctggcgcgcgtgacgtgatccgccgtgaggatttcgccaaaaccggcgcaaccaccatgcgtgaggtacttaaccgcatccctggcgtcagcgcgccggaaaacaacggcaccggcagccacgacctggcgatgaactttggcatccggggcctgaacccgcgcctcaccagccgctcgaccgtcctgatggacggcatccccgtccccttcgccccttacggtcagccgcagctttcactggctcccgtttcgctcggcaacatggatgccattgacgtggtacgcggtggtggtgcggtgcgttacggaccgcagagcgtgggcggcgtggtgaactttgttacccgtgccattccgcaggactttggtatcgaggcgggcgtggaaggtcagctcagcccaacctcttcacaaaacaacccgaaagagacgcacaacctgatggtgggcggcacagcggacaacggttttggcaccgcgctgctctactccggcacgcgcggcagtgacgggcgcgagcacagcgccacccgcatcgacgacctgatgctgaaaagcaaatatgcgccggatgaggtgcacaccttcaacagcctgctgcaatattacgacggtgaagccgacatgcccggtggcctgtctcgcgcggattacgacgccgatcgctggcaatccacccgcccgtatgaccgcttctggggtcgtcgcaagctggcgagcctgggctaccagttccagccagacagccagcataaattcaacattcaggggttctacacccaaaccctgcgcagcggctacctggagcaaggcaaacgcatcaccctctcgccgcgtaactactgggtgcgcggtattgagccacgctacagccagatctttatgatcggcccttccgcgcacgaagtgggcgtgggctatcgctatgtgaatgaatcaacgcatgaaatgcgttactacaccgccaccagcagcgggcagttgccgtccggctcaagcccttacgaccgcgatacgcgttccggcaccgaggcgcacgcctggtatctggatgacaaaatcgacatcggcaactggaccatcacgccgggtatgcgtttcgaacatatcgagtcataccagaacaacgccatcacaggcacgcacgaagaagtgagctataacgcaccgcttccggcgttgaacgtgctctatcacctgactgacagctggaatctttatgcaaacactgaaggctcgttcggcaccgtacagtacagccagattggcaaggctgtgcaaagcggcaatgtggaaccggaaaaagcgcgaacctgggaactcggtacccgctacgacgacggcgcgctgacggcggaaatggggctgttcctgattaactttaacaatcagtacgactccaaccagaccaacgacaccgtcactgcacgtggcaaaacgcgccataccgggctggaaacgcaggcacgttacgatctgggtacgctaacgccaacgcttgataacgtttccatctacgccagctatgcgtatgtgaacgcggaaatccgcgagaaaggcgacacctacggcaatctggtaccattctccccgaaacataaaggcacgctgggcgtggactacaagccaggaaactggacgttcaatctgaacagcgatttccagtccagccagtttgcggataacgccaatacggtgaaagagagcgccgacggcagtaccggccgcattcccggcttcatgctctggggcgcacgcgtggcgtatgactttggcccgcagatggcagatctgaacctggcattcggtgtgaaaaacatcttcgaccaggactacttcatccgctcttatgacgacaacaacaaaggcatctatgcaggccagccgcgcacgctgtatatgcaggggtcgttgaagttctga

SEQ ID NO: 10 - protein, 741 aa, Escherichia coli
AQVNIAPGSLDKALNQYAAHSGFTLSVDASLTRGKQSNGLHGDYDVESGLQQLLDGSGLQVKPLGNNSWTLEPAPAPKEDALTVVGDWLGDARENDVFEHAGARDVIRREDFAKTGATTMREVLNRIPGVSAPENNGTGSHDLAMNFGIRGLNPRLTSRSTVLMDGIPVPFAPYGQPQLSLAPVSLGNMDAIDVVRGGGAVRYGPQSVGGVVNFVTRAIPQDFGIEAGVEGQLSPTSSQNNPKETHNLMVGGTADNGFGTALLYSGTRGSDGREHSATRIDDLMLKSKYAPDEVHTFNSLLQYYDGEADMPGGLSRADYDADRWQSTRPYDRFWGRRKLASLGYQFQPDSQHKFNIQGFYTQTLRSGYLEQGKRITLSPRNYWVRGIEPRYSQIFMIGPSAHEVGVGYRYVNESTHEMRYYTATSSGQLPSGSSPYDRDTRSGTEAHAWYLDDKIDIGNWTITPGMRFEHIESYQNNAITGTHEEVSYNAPLPALNVLYHLTDSWNLYANTEGSFGTVQYSQIGKAVQSGNVEPEKARTWELGTRYDDGALTAEMGLFLINFNNQYDSNQTNDTVTARGKTRHTGLETQARYDLGTLTPTLDNVSIYASYAYVNAEIREKGDTYGNLVPFSPKHKGTLGVDYKPGNWTFNLNSDFQSSQFADNANTVKESADGSTGRIPGFMLWGARVAYDFGPQMADLNLAFGVKNIFDQDYFIRSYDDNNKGIYAGQPRTLYMQGSLKF

SEQ ID NO: 11 - DNA, 2202 bp, Escherichia coli
  source 1..2202: mol_type = genomic DNA
atgatgataagcaaaaagtatacgctttgggctctcaacccactgcttcttaccatgatggcgccagcagtcgctcaacaaaccgatgatgaaacgttcgtggtgtctgccaaccgcagcaatcgcaccgtagcggagatggcgcaaaccacctgggttatcgaaaacgccgaactggaacagcagattcagggcggcaaagagcttaaagacgcactggctcagctgatccctggccttgacgtcagcagccggagccgcaccaactacggtatgaatgtgcgtggccgcccgctggtcgtgctggttgacggcgtgcgtctcaactcttcacgtaccgacagccgacaactggactctatagatccttttaatatcgaccatattgaagtgatctccggtgcgacgtccctgtacggcggcggcagtaccggtggcctgatcaacatcgtgaccaaaaaaggccagccggaaaccatgatggagtttgaggctggcaccaaaagtggctttagcagcagtaaagatcacgatgaacgcattgccggagctgtctccggcggaaatgagcatatctccggacgtctttccgtggcatatcagaaatttggcggctggtttgacggtaacggcgatgccaccttgcttgataacacccagaccggcctgcagtactccgatcggctggacatcatgggaactggtacgctgaacatcgatgaatcccggcagcttcagttgatcacacagtactataaaagccagggcgacgacgattacgggcttaatctcgggaaaggcttctctgccatcagagggaccagcacgccattcgtcagtaacgggctgaattccgaccgtattcccggcactgagcggcatttgatcagcctgcagtactctgacagcgcttttctgggacaggagctggtcggtcaggtttactaccgcgatgagtcgttgcgattctacccgttcccgacggtaaatgcgaacaaacaggtgacggctttctcttcgtcacagcaggacaccgaccagtacggcatgaaactgactctgaacagcaaaccgatggacggctggcaaatcacctgggggctggatgctgatcatgagcgctttacctccaaccagatgttcttcgacctggctcaggcaagcgcttccggagggctgaacaacaagaagatttacaccaccgggcgctatccgtcgtatgacatcaccaacctggcggccttcctgcaatcaggctatgacatcaataatctctttaccctcaacggtggcgtacgctatcagtacactgaaaacaagattgatgatttcatcggctacgcgcagcaacggcagattgccgccgggaaggctacatccgccgacgccattcctggcggctcagtcgattacgacaacttcctgttcaacgccggtctgctgatgcacatcaccgaacgccagcaggcatggctcaacttctcccagggcgtggagctgccggacccgggtaaatactatggtcgcggcatctatggtgctgcagtgaacggccatcttcctctaacaaagagtgtgaacgtcagcgacagcaagctggaaggcgtgaaagtcgattcttatgagctgggctggcgctttactggcaataatctgcgtacccaaatcgcggcctactattcgatttctgataagagcgtggtggcgaataaagatctgaccatcagcgtggtggacgacaaacgccgtatttacggcgtggaaggtgcggtggactacctgattcctgatactgactggagtaccggagtgaacttcaacgtgctgaaaactgagtcgaaagtgaacggtacctggcagaaatacgatgtgaagacagcaagcccatcaaaagcgacagcctacattggctgggcaccggacccgtggagtctgcgcgtgcagagcaccacctcctttgacgtgagcgacgcgcagggctacaaggtcgatggctataccaccgtggatctgctcggcagttatcagcttccggtgggtacactcagcttcagcattgaaaaccttttcgaccgtgactacaccactgtctgggggcagcgtgcaccactgtactacagcccgggttacggcccagcgtcactgtacgactacaaaggcaggggccgaacctttggtctgaactactctgtgctgttctga

SEQ ID NO: 12 - protein, 707 aa, Escherichia coli
QQTDDETFVVSANRSNRTVAEMAQTTWVIENAELEQQIQGGKELKDALAQLIPGLDVSSRSRTNYGMNVRGRPLVVLVDGVRLNSSRTDSRQLDSIDPFNIDHIEVISGATSLYGGGSTGGLINIVTKKGQPETMMEFEAGTKSGFSSSKDHDERIAGAVSGGNEHISGRLSVAYQKFGGWFDGNGDATLLDNTQTGLQYSDRLDIMGTGTLNIDESRQLQLITQYYKSQGDDDYGLNLGKGFSAIRGTSTPFVSNGLNSDRIPGTERHLISLQYSDSAFLGQELVGQVYYRDESLRFYPFPTVNANKQVTAFSSSQQDTDQYGMKLTLNSKPMDGWQITWGLDADHERFTSNQMFFDLAQASASGGLNNKKIYTTGRYPSYDITNLAAFLQSGYDINNLFTLNGGVRYQYTENKIDDFIGYAQQRQIAAGKATSADAIPGGSVDYDNFLFNAGLLMHITERQQAWLNFSQGVELPDPGKYYGRGIYGAAVNGHLPLTKSVNVSDSKLEGVKVDSYELGWRFTGNNLRTQIAAYYSISDKSVVANKDLTISVVDDKRRIYGVEGAVDYLIPDTDWSTGVNFNVLKTESKVNGTWQKYDVKTASPSKATAYIGWAPDPWSLRVQSTTSFDVSDAQGYKVDGYTTVDLLGSYQLPVGTLSFSIENLFDRDYTTVWGQRAPLYYSPGYGPASLYDYKGRGRTFGLNYSVL

SEQ ID NO: 13 - protein, 660 aa, Escherichia coli
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 14 - protein, 682 aa, Escherichia coli
MKNKYIIAPGIAVMCSAVISSGYASSDKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREIKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 15 - protein, 725 aa, Escherichia coli
MRTHKLLWPLTALLVGFNSQVSVAKSSDDDNDETLVVEATAEQILKQQPGVSVITSEDIKKSPPVNDISDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSDEGATRRANFSLSGPLAGNALTTRLYGNLNKTDADSWDINSSAGTKNAAGHEGVRNKDINGVVSWKLNPQQILDFEAGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRYISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKDRLNWNTNATWMITSEQKDTGNPLSVIPKYTINNSLNWTITQAFSASVNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 16 - protein, 746 aa, Escherichia coli
MNKKIHSLALLVNLGIYGVAQAQEPTDTPVSHDDTIVVTAAEQNLQAPGVSTITADEIRKNPVARDVSEIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDAYTRSKYGDETNRLYRQNYSLTWNGGWDNGVTTSNWVQYEHTRNSRIPEGLAGGTEGKFNEKAAQDFVDIDLDDVMLHSEVNLPIDFLVNQTLTLGTEWNQQRMKDLSSNTQALTGTNTGGAIDGVSATDRSPYSKAEIFSLFAENNMELTDSTIVTPGLRFDHHSIVGNNWSPALNISQGLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSINTHF

SEQ ID NO: 17 - protein, 774 aa, Escherichia coli
MTPLRVFRKTTPLVNAIRLSLLPLAGLSFSAFAAQVNIAPGSLDKALNQYAAHSGFTLSVDASLTRGKQSNGLHGDYDVESGLQQLLDGSGLQVKPLGNNSWTLEPAPAPKEDALTVVGDWLGDARENDVFEHAGARDVIRREDFAKTGATTMREVLNRIPGVSAPENNGTGSHDLAMNFGIRGLNPRLTSRSTVLMDGIPVPFAPYGQPQLSLAPVSLGNMDAIDVVRGGGAVRYGPQSVGGVVNFVTRAIPQDFGIEAGVEGQLSPTSSQNNPKETHNLMVGGTADNGFGTALLYSGTRGSDGREHSATRIDDLMLKSKYAPDEVHTFNSLLQYYDGEADMPGGLSRADYDADRWQSTRPYDRFWGRRKLASLGYQFQPDSQHKFNIQGFYTQTLRSGYLEQGKRITLSPRNYWVRGIEPRYSQIFMIGPSAHEVGVGYRYVNESTHEMRYYTATSSGQLPSGSSPYDRDTRSGTEAHAWYLDDKIDIGNWTITPGMRFEHIESYQNNAITGTHEEVSYNAPLPALNVLYHLTDSWNLYANTEGSFGTVQYSQIGKAVQSGNVEPEKARTWELGTRYDDGALTAEMGLFLINFNNQYDSNQTNDTVTARGKTRHTGLETQARYDLGTLTPTLDNVSIYASYAYVNAEIREKGDTYGNLVPFSPKHKGTLGVDYKPGNWTFNLNSDFQSSQFADNANTVKESADGSTGRIPGFMLWGARVAYDFGPQMADLNLAFGVKNIFDQDYFIRSYDDNNKGIYAGQPRTLYMQGSLKF

SEQ ID NO: 18 - protein, 732 aa, Escherichia coli
MMISKKYTLWALNPLLLTMMAPAVAQQTDDETFVVSANRSNRTVAEMAQTTWVIENAELEQQIQGGKELKDALAQLIPGLDVSSRSRTNYGMNVRGRPLVVLVDGVRLNSSRTDSRQLDSIDPFNIDHIEVISGATSLYGGGSTGGLINIVTKKGQPETMMEFEAGTKSGFSSSKDHDERIAGAVSGGNEHISGRLSVAYQKFGGWFDGNGDATLLDNTQTGLQYSDRLDIMGTGTLNIDESRQLQLITQYYKSQGDDDYGLNLGKGFSAIRGTSTPFVSNGLNSDRIPGTERHLISLQYSDSAFLGQELVGQVYYRDESLRFYPFPTVNANKQVTAFSSSQQDTDQYGMKLTLNSKPMDGWQITWGLDADHERFTSNQMFFDLAQASASGGLNNKKIYTTGRYPSYDITNLAAFLQSGYDINNLFTLNGGVRYQYTENKIDDFIGYAQQRQIAAGKATSADAIPGGSVDYDNFLFNAGLLMHITERQQAWLNFSQGVELPDPGKYYGRGIYGAAVNGHLPLTKSVNVSDSKLEGVKVDSYELGWRFTGNNLRTQIAAYYSISDKSVVANKDLTISVVDDKRRIYGVEGAVDYLIPDTDWSTGVNFNVLKTESKVNGTWQKYDVKTASPSKATAYIGWAPDPWSLRVQSTTSFDVSDAQGYKVDGYTTVDLLGSYQLPVGTLSFSIENLFDRDYTTVWGQRAPLYYSPGYGPASLYDYKGRGRTFGLNYSVL

SEQ ID NO: 19 - DNA, 1890 bp, Escherichia coli
  source 1..1890: mol_type = genomic DNA
ttgatgaaacctgcggcatccttcttctattgtggatgctttacaatgattaaaaaagcttcgctgctgacggcgtgttccgtcacggcattttccgcttgggcacaggataccagcccggatactctcgtcgttactgctaaccgttttgaacaaccgcgcagcactgtgcttgcgccaactactgttgtgacgcgtcaggatatcgaccgctggcagtcgacctcggtcaatgatgtgctgcgccgtcttccgggcgtggatatcacccaaaacggcggttcaggtcagctctcatctatttttattcgcggtacaaatgccagtcatgtgttggtgttaattgatggcgtccgcctgaatttggcgggggtgagtggttctgctgatcttagtcagttccctattgcgcttgtccagcgtgttgaatatatccgtgggccgcgctccgctgtttatggttcagatgcaataggcggggtcgtgaatatcatcacgacgcgcgatcaccccggaacggaaatttcagcagggtggggaagcaatagttatcagaactatgatgtctctacacagcaacaactgggggataagacacgggtaacgctgttgggcgattatgcccatactcatggttatgatgttgtcgcctatggtaataccggaacgcaagcgcagccagataacgatggttttttaagtaaaacgctttatggcgcgctggagcataattttactgatgcctggagcggctttgtgcgcggctatggctatgataatcgtaccaattatgacgcatattattctcccggcttgccactggtcgatacccgtaaactctatagccaaagttgggatgccggactgcgctataacggcgaactgattaaatcacaactgattaccagctacagccatagcaaagattacaactacgatccgcattatggtcgttatgattcgtccgcgacgctcgatgagatgaaacagtacaccgtccagtgggcaaacaacatcatcattggccacggtaatattggtgcgggtgttgactggcagaagcagagcacggcaccgggcacagcttatgttgaggatggatatgatcaacgtaataccggcatctatctgactgggctgcagcaagtcggcgattttacttttgaaggcgcaggacgcagcgacgataattctcagtttggtcgtcatggtacctggcaaaccagcgccggttgggagttcatcgaaggttatcgctttattgcttcctacggaacatcttataaggcaccaaatcttgggcaactgtacggctcctacggtaatccgaatctgaatcctgagaaaagcaaacaatgggaaggcgcgtttgaaggcttaactgctggggttaactggcgtatttccggatatcgtaacgatgtcagtgacttgatcgattatgatgatcacaccctgaaatattacaatgaagggaaagcgcggattaagggtgtcgaggcgaccgctaattttgataccggaccactgacgcatactgtgagttatgattatgttgatgcgcgcaatgcgattactgacacgccgttgttacgccgtgctaaacagcaggtgaaataccagctcgactggcagttgtatgacttcgactgggggattacttatcagtatttaggcactcgctatgataaggattactcctcttatccttatcaaaccgttaaaatgggcggtgtgagcttgtgggatcttgcggttgcgtatccggtcacctctcacctgacagttcgtggtaaaatagccaacctgttcgacaaagattacgagacagtctatggctaccaaactgcaggacgggaatataccttgnctggcagctacaccttctga

SEQ ID NO: 20 - protein, 594 aa, Escherichia coli
QDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDHPGTEISAGWGSNSYQNYDVSTQQQLGDKTRVTLLGDYAHTHGYDVVAYGNTGTQAQPDNDGFLSKTLYGALEHNFTDAWSGFVRGYGYDNRTNYDAYYSPGLPLVDTRKLYSQSWDAGLRYNGELIKSQLITSYSHSKDYNYDPHYGRYDSSATLDEMKQYTVQWANNIIIGHGNIGAGVDWQKQSTAPGTAYVEDGYDQRNTGIYLTGLQQVGDFTFEGAGRSDDNSQFGRHGTWQTSAGWEFIEGYRFIASYGTSYKAPNLGQLYGSYGNPNLNPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLXGSYTF

SEQ ID NO: 21 - DNA, 1992 bp, Escherichia coli
  source 1..1992: mol_type = genomic DNA
atgtttaggttgaaccctttcgtacgggtcgggctgtgtttgtccgctatttcttgtgcatggcctgtgttagcggtcgatgatgatggcgaaacgatggttgtcactgcatcttccgttgaacaaaaccttaaagatgctcccgccagtatcagcgtcattacccaggaagacctgcagcgaaaaccggtacagaatctgaaggatgtcctcaaagaagtgcctggcgtacaactgacgaacgaaggggataaccgtaagggcgtaagtattcgtggtctggacagcagctacaccctgattcttgtcgacggtaaacgcgttaactcccgcaatgccgtcttccgccacaatgatttcgatctgaactggatcccggtcgattccatcgaacgtattgaagtggttcgtggcccgatgtcgtcgctgtacggttccgatgcgctcggcggtgtagtgaatatcatcaccaaaaaaatcggtcagaaatggtcgggcaccgttaccgtcgataccaccgttcaggaacatcgcgatcgcggtgatacctataacggtcaattctttaccagcggaccattaattgacggcgtgctgggaatgaaagcttacggcagcctggcaaaacgtgaaaaggatgacccgcaaaactcaacgaccaccgataccggagaaacgccgcgtattgaaggattctccagccgcgacggcaatgtcgaatttgcctggacaccgaatcaaaatcacgattttactgccggatacggtttcgaccgtcaggatcgtgattccgactcgctggacaaaaaccgcctggaacgccagaactactccgtcagccataatgggcgttgggattacggcaccagcgaactgaaatactacggtgagaaagtcgagaacaaaaaccctggcaacagcagcccgataacttccgaaagcaatacggtcgacggcaaatacacgttgccgctgacggcgattaatcagtttctcacggttggcggtgaatggcgtcacgacaaacttagcgatgcggtgaacctgaccgggggaaccagctccaaaacgtctgccagccagtacgcgctgtttgtggaagatgaatggcggatcttcgagccgctggcgctgacgaccggcgtgcgtatggacgatcacgaaacctacggtgaacactggagtccgcgtgcctacctggtttataacgccaccgacaccgtaacggtgaaagggggctgggcgacggcatttaaagccccttctctgttgcaacttagccctgactggacgagcaattcctgccgtggcgcatgtaagattgtgggtagcccggatctgaaaccagaaaccagcgaaagttgggagctggggctttactacatgggtgaagaaggctggctggaaggggttgaatccagcgttaccgttttccgtaacgatgtgaaagatcgtatcagcattagccgtacgtctgacgtcaatgctgcaccgggctaccaaaactttgtcggttttgagacgggcgctaacggacggcgcataccggtatttagctactacaacgttaacaaagctcgtattcagggcgtggaaaccgaactgaaaattccgttcaacgatgaatggaaactgtcgatcaactacacctacaacgatggtcgtgatgtcagcaacggcgaaaacaaaccgctatccgatctgccgttccatactgctaacggtacgctggactggaaaccgctggcgctggaagactggtcattctatgtttctggtcactataccgggcagaaacgcgccgacagcgcgacggctaaaacaccgggcggttataccatctggaataccggcgcggcctggcaggtgactaaagacgtcaaactgcgcgcaggcgtgctgaaccttggcgacaaggatctcagtcgtgacgactacagctataacgaagacggacgtcgttactttatggcagtggattatcgcttctga

SEQ ID NO: 22 - protein, 638 aa, Escherichia coli
VDDDGETMVVTASSVEQNLKDAPASISVITQEDLQRKPVQNLKDVLKEVPGVQLTNEGDNRKGVSIRGLDSSYTLILVDGKRVNSRNAVFRHNDFDLNWIPVDSIERIEVVRGPMSSLYGSDALGGVVNIITKKIGQKWSGTVTVDTTVQEHRDRGDTYNGQFFTSGPLIDGVLGMKAYGSLAKREKDDPQNSTTTDTGETPRIEGFSSRDGNVEFAWTPNQNHDFTAGYGFDRQDRDSDSLDKNRLERQNYSVSHNGRWDYGTSELKYYGEKVENKNPGNSSPITSESNTVDGKYTLPLTAINQFLTVGGEWRHDKLSDAVNLTGGTSSKTSASQYALFVEDEWRIFEPLALTTGVRMDDHETYGEHWSPRAYLVYNATDTVTVKGGWATAFKAPSLLQLSPDWTSNSCRGACKIVGSPDLKPETSESWELGLYYMGEEGWLEGVESSVTVFRNDVKDRISISRTSDVNAAPGYQNFVGFETGANGRRIPVFSYYNVNKARIQGVETELKIPFNDEWKLSINYTYNDGRDVSNGENKPLSDLPFHTANGTLDWKPLALEDWSFYVSGHYTGQKRADSATAKTPGGYTIWNTGAAWQVTKDVKLRAGVLNLGDKDLSRDDYSYNEDGRRYFMAVDYRF

SEQ ID NO: 23 - protein, 629 aa, Escherichia coli
MMKPAASFFYCGCFTMIKKASLLTACSVTAFSAWAQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDHPGTEISAGWGSNSYQNYDVSTQQQLGDKTRVTLLGDYAHTHGYDVVAYGNTGTQAQPDNDGFLSKTLYGALEHNFTDAWSGFVRGYGYDNRTNYDAYYSPGLPLVDTRKLYSQSWDAGLRYNGELIKSQLITSYSHSKDYNYDPHYGRYDSSATLDEMKQYTVQWANNIIIGHGNIGAGVDWQKQSTAPGTAYVEDGYDQRNTGIYLTGLQQVGDFTFEGAGRSDDNSQFGRHGTWQTSAGWEFIEGYRFIASYGTSYKAPNLGQLYGSYGNPNLNPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLXGSYTF

SEQ ID NO: 24 - protein, 690 aa, Escherichia coli
MLPYARGRKEDVSDTHFIFVVTSWRYGMFRLNPFVRVGLCLSAISCAWPVLAVDDDGETMVVTASSVEQNLKDAPASISVITQEDLQRKPVQNLKDVLKEVPGVQLTNEGDNRKGVSIRGLDSSYTLILVDGKRVNSRNAVFRHNDFDLNWIPVDSIERIEVVRGPMSSLYGSDALGGVVNIITKKIGQKWSGTVTVDTTVQEHRDRGDTYNGQFFTSGPLIDGVLGMKAYGSLAKREKDDPQNSTTTDTGETPRIEGFSSRDGNVEFAWTPNQNHDFTAGYGFDRQDRDSDSLDKNRLERQNYSVSHNGRWDYGTSELKYYGEKVENKNPGNSSPITSESNTVDGKYTLPLTAINQFLTVGGEWRHDKLSDAVNLTGGTSSKTSASQYALFVEDEWRIFEPLALTTGVRMDDHETYGEHWSPRAYLVYNATDTVTVKGGWATAFKAPSLLQLSPDWTSNSCRGACKIVGSPDLKPETSESWELGLYYMGEEGWLEGVESSVTVFRNDVKDRISISRTSDVNAAPGYQNFVGFETGANGRRIPVFSYYNVNKARIQGVETELKIPFNDEWKLSINYTYNDGRDVSNGENKPLSDLPFHTANGTLDWKPLALEDWSFYVSGHYTGQKRADSATAKTPGGYTIWNTGAAWQVTKDVKLRAGVLNLGDKDLSRDDYSYNEDGRRYFMAVDYRF

SEQ ID NO: 25 - DNA, 2241 bp, Escherichia coli
  source 1..2241: mol_type = genomic DNA
atgaacaagaagattcattccctggccttgttggtcaatctggggatttatggggtagcacaggcacaagagccgaccgatactcctgtttcacatgacgataccattgtcgttaccgccgccgagcagaacttgcaggcgcctggcgtttcgaccattaccgcagatgaaatccgcaaaaacccggttgcccgcgatgtatcggagatcattcgtaccatgcctggcgttaacctgaccggtaactccaccagtggtcagcgtggtaataaccgccagattgatattcgcggcatgggtccggaaaacacgctgattttgattgacggcaagccggtaagcagccgtaactctgtgcgtcagggctggcgtggcgagcgcgatacccgtggtgatacctcctgggtgccgcctgaaatgattgaacgtattgaagttctgcgtggtccggcagctgcgcgttatggcaacggcgcggcgggcggcgtggttaacatcattaccaaaaaaggcagcggtgagtggcacggctcctgggatgcttatttcaatgcgccagaacataaagaggaaggtgccaccaaacgcaccaacttcagcctgaccggtccgctgggcgacgaattcagcttccgcttgtatggcaacctcgacaaaacccaggctgacgcgtgggatatcaaccagggccatcagtccgcgcgtgccggaacgtatgccacgacgttaccagccgggcgcgaaggggtgatcaacaaagatattaatggcgtggtgcgctgggacttcgcgcctctgcagtcactcgaactggaagcgggctacagccgccagggtaacctgtatgcgggtgatacgcagaacaccaactctgacgcttacactcgatcgaaatatggcgatgaaaccaaccgcctgtatcgccagaactactcgctgacctggaacggtggctgggataacggcgtgaccaccagcaactgggtgcagtacgaacacacccgtaactcgcgtattccggaaggtctggcgggcggtaccgaagggaaatttaacgaaaaagcggcacaggattttgtagatatcgatcttgatgacgtgatgctgcacagcgaagttaacctgccgattgatttcctcgtaaaccaaacgctgacgctgggtacagagtggaatcagcaacggatgaaggacttaagttccaacacccaggcgctgaccgggacgaataccggcggtgctattgatggtgtgagtgccaccgaccgtagcccgtattcaaaagcagaaattttctcgctgtttgccgaaaacaatatggagctgactgacagcaccatcgtaacgccggggctgcgtttcgatcatcacagtattgtcggcaataactggagcccggcgctgaacatatcgcaaggtttaggcgatgacttcacgctgaaaatgggcatcgcccgcgcctataaagcgccgagcctgtaccagactaacccaaactacattctctacagtaaaggtcagggctgctatgccagcgcgggcggctgctatctgcaaggtaatgatgacctgaaagcagaaaccagcatcaacaaggagattggtctggagttcaaacgcgacggttggctggcgggcgtgacctggttccgtaacgattatcgcaataagattgaagcgggctatgtggctgtagggcaaaacgcagtcggcaccgatctctatcagtgggataacgtaccgaaagcggtggttgaaggtctggaaggatcgttaaacgtaccggttagcgaaacggtgatgtggaccaataacatcacttatatgctgaagagtgaaaacaaaaccacgggcgaccgtttgtcgatcatcccggagtatacgttgaactcaacgctgagctggcaggcacgggaagatttgtcgatgcaaacgaccttcacctggtacggcaaacagcagccgaagaagtacaactataaaggtcagccagcggttggaccggaaaccaaagaaatcagtccgtacagcattgttggcctgagcgcgacctgggatgtgacgaagaatgtcagtctgaccggcggcgtggacaacctgttcgacaaacgtttgtggcgtgcgggtaatgcccagaccacgggcgatctggcaggggccaactatatcgccggtgccggtgcgtatacctataacgagccgggacgtacgtggtatatgagcattaatactcacttctga

SEQ ID NO: 26 - protein, 724 aa, Escherichia coli; identical to SEQ ID NO: 8

SEQ ID NO: 27 - protein, 746 aa, Escherichia coli; identical to SEQ ID NO: 16

SEQ ID NO: 28 - DNA, 2178 bp, Escherichia coli
  source 1..2178: mol_type = genomic DNA
atgagaattaacaaaatcctctggtcgctaactgtgctcctagttgggttgaatagccaggtatcagtagccaaatactccgacgatgataatgacgagactctggtggtggaagccaccgctgagcaggtattaaaacagcagccgggcgtgtcggttattaccagcgaggatattaaaaagacccctccggtaaacgacctttcagatattattcgtaaaatgcctggtgttaatcttaccggcaatagcgcctcgggcacacgcggtaataaccgccagatcgatattcgtggtatggggccggaaaacaccttaattttaattgatggtgtaccggtgacgtcacgtaactccgtgcgttatagctggcgtggggagcgtgatacccgcggtgacaccaactgggtgccaccggaacaggttgagcgtattgaagtgatccgcggccctgcggcggcgcgctacggttcgggggccgccgggggggtggtgaacatcattaccaaacgtcccaccaacgactggcacggttcgctgtcgttatacaccaaccagccggaaagtagcgaagagggcgctacgcgtcgcgccaatttcagccttagtgggcctctggctggtgatgctcttaccacgcgtttgtatggtaacctgaataaaacggatgctgacagttgggatattaattctccggtcggtacgaaaaacgcagccgggcatgaaggggtacgtaacaaagatattaacggcgttgtctcgtggaaattaaatccgcagcagattctcgatttcgaagtcggatatagccgccaggggaatatctatgcgggcgatacgcagaacagttcttccagtgcagttaccgaaagcctggcaaaatccggcaaagagacgaaccgcctgtaccgacagaattatggcattacgcataatggtatctgggactggggacaaagtcgctttggtgtttattacgagaaaaccaataatacccgcatgaatgaaggattatccggcggtggtgaaggacgtattttagcgggtgaaaagtttacgaccaatcgcctgagttcctggcgaaccagcggtgagcttaatattcctttgaatgtgatggttgatcaaacgctgaccgttggtgcagagtggaaccgcgataagctcgatgatccttcctctaccagcctgacggtgaatgacagagatatcagcggtatttctggctctgctgcggatcgcagcagtaaaaatcattctcaaatcagtgcgctgtatattgaagataacattgagccggttcctggcacgaatatcattcccggcctgcgctttgattatctcagcgactccggcgggaacttcagccccagtctgaatctttcgcaggaattgggcgattatttcaaagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattggactggagttcacctgggaagattaccacgcaagtgtgacctacttccgcaatgattaccagaataagatcgtggccggggataacgttatcgggcaaaccgcttcaggcgcatatatcctcaagtggcagaatggcgggaaagctctggtggacggtatcgaagccagtatgtctttcccactggtgaaagagcgtctgaactggaataccaatgccacatggatgatcacttcggagcaaaaagacaccggtaatcctctgtcggtcatcccgaaatatactatcaataactcgcttaactggaccatcacccaggcgttttctgccagcttcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtcagtaatatcctcaataaacagatcttccgatcttctgaaggggcgaatacctataacgagccaggccgggcttattatgccggagttaccgcatcattctga

SEQ ID NO: 29 - protein, 701 aa, Escherichia coli
KYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVVSWKLNPQQILDFEVGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKERLNWNTNATWMITSEQKDTGNPLSVIPKYTINNSLNWTITQAFSASFNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 30 - protein, 725 aa, Escherichia coli
MRINKILWSLTVLLVGLNSQVSVAKYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVVSWKLNPQQILDFEVGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKERLNWNTNATWMITSEQKDTGNPLSVIPKYTINNSLNWTITQAFSASFNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 31 - DNA, 2049 bp, Escherichia coli
  source 1..2049: mol_type = genomic DNA
atgaagaacaaatatatcattgctccgggcattgccgtgatgtgttctgcagttatatcatcaggttatgccagttctgataaaaaagaagatacgcttgttgttactgcctccgggttcactcagcagctcagaaatgccccggccagtgtctcagtcattacttcagaacaactgcaaaaaaaaccggtttcagatctggtcgatgcagtaaaagatgttgaagggattagtatcactggtgggaatgaaaaaccggatatcagtatacgtggtctaagtggcgattacacgctgattctggtcgatggacgacgtcagagcggtcgggaatccagaccaaacggcagcggcggttttgaagccggatttatccctcctgtggaagcaattgaacgcattgaagtgatccgtggccctatgtcttccctgtatggttctgatgccatcggaggggtcattaatatcataaccaaaccagttaataaccaaacatgggatggcgtacttggacttggggggattattcaggaacatgggaaatttggtaactcaaccacaaatgacttctatctgtcaggcccattgattaaggataaacttggtcttcagctatatggaggaatgaactatcgcaaggaagatagtatctctcagggaacaccggcaaaagataataagaatataacggcaacgctccagtttactccgactgaaagccagaagtttgtttttgaatatggaaaaaataaccaggtgcatacattaacacctggtgagtctctcgatgcctggactatgcggggaaatcttaaacaaccaaacagtaaaagagaaacgcataattcacgtagtcactgggtagcagcatggaatgcccagggcgaaatactgcatcctgaaattgctgtttatcaggagaaagttattcgtgaggttaaatcaggtaaaaaagataaatataatcattgggatcttaattacgagtcaagaaaaccggaaataaccaacacaatcatagatgcaaaagtgacggcatttctgccggaaaatgtactgaccatcggaggtcaatttcagcatgcagagctccgtgatgactcagccacgggtaaaaaaacgacagaaacacagtctgtttcaattaaacagaaagctgtttttatagaaaatgaatatgcagcaacggattctctcgccctgactggaggactgcgtctcgataatcatgaaatctatggcagttactggaatccaagattgtacgctgtttataacctgaccgataatctcacactcaaaggggggatcgcaaaagcatttcgggctccttcaattcgtgaggtgagtcctggatttggaacactgacgcagggtggtgcctctattatgtatggaaacagggacctgaaaccggagaccagtgtaaccgaagagatcggtattatttatagtaatgatagtggtttttcggcgagcgcgacgctgtttaatactgattttaaaaataagttgaccagttacgatataggtacaaaagatccagtcaccgggttaaacacttttatttatgataatgtaggtgaggcaaatatcagaggggtggagcttgcaactcagattcctgtgtatgataaatggcatgtatctgcaaactatacatttactgactctcgtcgaaaaagtgatgacgaaagtctcaatggcaagtcgctgaaaggggaacctctggaaagaactcccagacatgcagccaatgcaaaactggaatgggattacactcaggatattacattttattcatctctgaattatacgggaaaacaaatctgggcagcacaaagaaatggtgctaaggttccccgcgttcgtaatggattcacatctatggatattggtctaaattaccagattctgccagacacgctgattaattttgccgttcttaacgtcacagacagaaagagcgaggatatcgataccattgatggtaactggcaggtcgatgaaggacgccgttattgggctaatgtaagagtatccttctga

SEQ ID NO: 32 - protein, 656 aa, Escherichia coli
DKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREVKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 33 - protein, 682 aa, Escherichia coli
MKNKYIIAPGIAVMCSAVISSGYASSDKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREVKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 34 - DNA, 1947 bp, Escherichia coli
  source 1..1947: mol_type = genomic DNA
atgttggctttggctgtttctgccaccttgccaacgtttgcttttgctactgaaaccatgaccgttacggcaacggggaatgcacgtagttccttcgaagcgcctatgatggtcagcgttatcgacacttccgctcctgaaaatcaaactgctacttcagccactgatttgctgcgtcatgttcctggaattactcttgatggtaccggacgaaccaacggtcaggatgtaaatatgcgtggctatgatcatcgcggcgtgctggttcttgtcgatggtgttcgccagggaacggataccggacacctgaatggcacttttctcgatccggcgctgatcaagcgtgttgagattgttcgcggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttgaagcttctgatagcagcaacccgatggtcgatcgttcaacaattcaacgcgatgcgcagctttcttataaactcgccccgcagggtaacgactggttaaatgcagatgcaaaaatttactggtcggaagtccgtattaatgcgcaaaacacggggagttcaggcgagtatcgtgaacagataacaaaaggagcaaggctggagaaccgttccactctatttgccgacagtttcgcttctcacttactgacatatggcggtgagtattatcgtcaggaacaacatccgggtggcgcgacgacgggcttcccgcaagcaaaaatcgattttagctctggttggctacaagatgagatcaccttacgcgatctgccgattaccctgcttggcggaacccgctatgacagttatcgcggtagcagcgacggctacaaagatgttgatgccgacaaatggtcatctcgtgcggggatgactatcaacccgaccaactggctgatgttatttggctcatatgctcaggcattccgcgccccgacgatgggcgaaatgtataacgattctaaacacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaagattatatctccacgaccgtcgatttcgcggcggcgacaactatgtcgtataacgtcccgaacgccaaaatctggggctgggatgtgatgacgaaatataccactgatctgtttagccttgatgtggcctataaccgtacccgcggcaaagacaccgataccggggaatatatctccagcattaacccggataccgttaccagtaccctgaatattccgatcgctcacagcggcttctctgttggttgggtcggtacgtttgccgatcgctcaacacatatcagcagcagctacagcaaacaacctggctatggtgtgaatgatttctacgtcagttatcaagggcagcaggcgctcaaaggcatgaccactactctggtattgggcaacgccttcgataaagagtactggtcgccgcaaggcatcccacaggatggtcgtaacggaaaaattttcgtgagttatcaatggtaa

SEQ ID NO: 35 - protein, 632 aa, Escherichia coli; identical to SEQ ID NO: 2

SEQ ID NO: 36 - protein, 648 aa, Escherichia coli
MLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 37 - DNA, 2280 bp, Escherichia coli
  source 1..2280: mol_type = genomic DNA
atggcgcagcggcagccggaaaaaaccgcagccggaggctgctgctttaactcactatacaacaaatattcaggaataacgatgatgcgcaaaaagtatatgccccgggctcttggtccgctgcttcttgtcgtgctgtcaccagctgtcgcccagcaaaacgatgataatgagatcatagtgtctgccagccgcagcaatcgaactgtagcggagatggcgcaaaccacctgggttatcgaaaatgccgaactggagcagcagattcagggcggtaaagagctgaaagacgcactggctcagttaatccccggccttgatgtcagcagccagagccgaaccaactacggtatgaacatgcgtggccgcccgctggttgtcctgattgacggtgtgcgcctcaactcttcacgttccgacagccgacaactggactctgtcgatccttttaatatcgaccatattgaagtgatctccggcgcgacggccctgtacggtggcgggagtaccggagggttgatcaacatcgtgaccaaaaaaggccagccggaaaccatgatggagtttgaggctggcacaaaaagtggctttaacagcagtaaagatcacgatgagcgcattgccggtgctgtctccggcggaaatgaccatatctccggacgtctttccgtggcatatcagaaatttggcggctggtttgacggtaacggcgatgccaccctgcttgataacacccagaccggcctgcagcactccaatcggctggacatcatgggaaccggtacgctgaacatcgatgaatcccggcagcttcaactgataacgcagtactataaaagtcagggggacgacaattacgggcttaatctcgggaaaggcttttccgccatcagcgggagcagcacaccatacgtcagtaaggggctgaattctgaccgcattcccggcactgagcggcatttgatcagcctgcagtactctgacagtgatttcctgagacaggaactggtcggtcaggtttactaccgcgatgagtcgttgcggttctacccgttcccgacggtaaatgcgaataaacaggcgacggctttctcctcgtcacagcaggataccgaccagtacggcatgaaactgactctgaacagccaacttatggacggctggcaaatcacctgggggctggatgctgagcatgagcgctttacctccaaccagatgttcttcgatctggctcaggcaagtgcttccggagggctgaacaaccataagatttacaccaccgggcgctatccgtcatatgacatcaccaatctggcggccttcctgcaatccagctatgacattaatgatatttttaccgttagcggtggcgtacgctatcagtatactgagaacagggtagatgatttcatcgactacacgcagcaacagaagattgctgccgggaaggcgatatctgccgacgccattcctggtggttcggtagattacgataactttctgttcaatgctggtctgctgatgcacatcaccgaacgtcagcaggcatggttcaatttttcccagggggtggcattgccggatccggggaaatattatggtcgcggcatctatggtgcagcagtgaacggccatcttcccctgacaaagagcgtgaacgtcagcgacagtaagctggaaggcgtgaaagtcgattcttatgaactgggctggcgctttaccggtgacaacctgcggactcaaatcgcggcatattactcgctttccaataagagcgtggaaaggaataaagatctgaccatcagtgtgaaggacgacaggcgccgtatttacggcgtggaaggtgcggtggactacctgatcccggatactgactggagtaccggtgtgaacttcaatgtgctgaaaaccgagtcgaaagtgaacggtcaatggcaaaaatatgacgtgaaggaatcaagtccatcgaaagcgacagcttacattaactgggcgccggaaccgtggagtctgcgtgtacagagcaccacttctttcgacgtaagcgatgcagagggtaacgatattaatggttacactaccgtcgattttatcagtagttggcagcttccggtgggaacactcagcttcagcgttgagaacctcttcgaccgtgactataccactgtctggggacagcgtgcacctctgtactacagcccgggttacggccctgcttcactgtacgactacaaaggccggggccgaacctttggtctgaactactcagtgctgttctga

SEQ ID NO: 38 - protein, 708 aa, Escherichia coli
QQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 39 - protein, 759 aa, Escherichia coli
MAQRQPEKTAAGGCCFNSLYNKYSGITMMRKKYMPRALGPLLLVVLSPAVAQQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 40 - DNA, 2325 bp, Escherichia coli
  source 1..2325: mol_type = genomic DNA
atgacgccgttacgcgtttttcgtaaaacaactcctttggttaacgccattcgcctgagcctgctgccgctggccggtctctcgttttccgcttttgctgcacaggttgatatcgcaccgggatcgctcgacaaagcgctcaatcagtatgccgcacacagcggaattaccctctcggttgacgccagcctgacgcgcggcaagcagagcaacggcctgcacggagattacgacgtcgagagcggcctgcaacagctgctggacggcagcggactgcaggtaaaaccgctgggaaataacagctggacgctggagcccgcgcccgcgccaaaagaagatgccctgaccgtggtcggcgactggctgggcgatgcgcgtgaaaacgacgtatttgaacatgctggcgcgcgtgacgtgatccgccgtgaggatttcgccaaaaccggcgcaaccaccatgcgtgaggtgcttaaccgcatccctggcgtcagcgcgccggaaaacaacggcaccggcagccacgacctggcgatgaactttggcatccggggcctgaacccacgcctcgccagccgctcgaccgtcctgatggacggcatccccgtcccctttgccccttacggtcagccgcagctttcactggctcccgtttcgctcggcaacatggatgccattgacgtggtgcgcggtggtggtgcggtgcgttacggaccgcagagcgtgggcggcgtggtgaactttgttacccgcgccattccgcaggactttggtatcgaggcgggggtggaaggtcagctcagcccaacctcttcacaaaacaacccgaaagagacgcacaacctgatggtgggcggcacagcggacaacggttttggcaccgcgctgctctactccggcacgcgcggcagtgactggcgcgagcacagcgccacccgcatcgacgacctgatgctgaaaagcaaatatgcgccgaatgaggtgcacaccttcaacagcctgctgcaatattacgatggtgaagccgacatgcccggcggcctgtcccgcgcggattacgacgccgatcgctggcaatccacccgcccgtatgaccgcttctggggccgtcgcaagctggcgagcctgggctaccagttccagccggacagccagcataaattcaacattctggggttctacacccaaaccctgcgcagcggctacctggagcaaggcaaacgcatcaccctctcgccgcgtaactactgggtgcgcggtattgagccacgctacagccagagctttatgatcggcccttccgcgcacgaagtgggcgtgggctatcgctatgtgaatgaatcaacgcatgaaatgcgttactacaccgccaccagcagcgggcagttgccgtccggctcaagcccttacgaccgcgacacgcgttccggcaccgaggcgcacgcctggtatctggatgacaaaatcgacatcggcaactggaccatcacgccgggtatgcgtttcgaacatatcgagtcataccagaacaacgccatcaaaggcacgcacgaagaggtaagctataacgcaccgcttccggcgttgaacgtgctctatcacctgactgacagctggaatctttatgcaaacactgaaggctcgttcggcaccgtacagtacagccagattggcaaggctgtgcaaagcggcaatgtggaaccggaaaaagcgcgaacctgggaactcggtacccgctacgacgacggcgcgctgacggcggaaatggggctgttcctgattaactttaacaatcagtacgactccaaccagaccaacgacaccgtcactgcacgtggcaaaacgcgccataccgggctggaaacgcaggcacgttacgacctgggtacgctaacgccaacgcttgataacgtttccgtctacgccagctatgcgtatgtgaacgcggaaatccgcgagaaaggcgacacctatggcaatcaggtgccattctccccgaaacataaaggcacgctgggcgtggactacaagccgggcaactggacgttcaatctgaacagcgatttccagtccagccagtttgcggataacgccaatacggtgaaagagagcgccgacggcagtaccggccgcattcccggcttcatgctctggggcgcacgcgtggcgtatgactttggcccgcagatggcagatctgaacctggcgttcggtgtgaaaaacatcttcgaccaggactacttcatccgctcttatgacgacaacaacaaaggcatctacgcaggccagccgcgcacgctgtatatgcaggggtcgttgaagttctga

SEQ ID NO: 41 - protein, 741 aa, Escherichia coli
AQVDIAPGSLDKALNQYAAHSGITLSVDASLTRGKQSNGLHGDYDVESGLQQLLDGSGLQVKPLGNNSWTLEPAPAPKEDALTVVGDWLGDARENDVFEHAGARDVIRREDFAKTGATTMREVLNRIPGVSAPENNGTGSHDLAMNFGIRGLNPRLASRSTVLMDGIPVPFAPYGQPQLSLAPVSLGNMDAIDVVRGGGAVRYGPQSVGGVVNFVTRAIPQDFGIEAGVEGQLSPTSSQNNPKETHNLMVGGTADNGFGTALLYSGTRGSDWREHSATRIDDLMLKSKYAPNEVHTFNSLLQYYDGEADMPGGLSRADYDADRWQSTRPYDRFWGRRKLASLGYQFQPDSQHKFNILGFYTQTLRSGYLEQGKRITLSPRNYWVRGIEPRYSQSFMIGPSAHEVGVGYRYVNESTHEMRYYTATSSGQLPSGSSPYDRDTRSGTEAHAWYLDDKIDIGNWTITPGMRFEHIESYQNNAIKGTHEEVSYNAPLPALNVLYHLTDSWNLYANTEGSFGTVQYSQIGKAVQSGNVEPEKARTWELGTRYDDGALTAEMGLFLINFNNQYDSNQTNDTVTARGKTRHTGLETQARYDLGTLTPTLDNVSVYASYAYVNAEIREKGDTYGNQVPFSPKHKGTLGVDYKPGNWTFNLNSDFQSSQFADNANTVKESADGSTGRIPGFMLWGARVAYDFGPQMADLNLAFGVKNIFDQDYFIRSYDDNNKGIYAGQPRTLYMQGSLKF

SEQ ID NO: 42 - protein, 774 aa, Escherichia coli
MTPLRVFRKTTPLVNAIRLSLLPLAGLSFSAFAAQVDIAPGSLDKALNQYAAHSGITLSVDASLTRGKQSNGLHGDYDVESGLQQLLDGSGLQVKPLGNNSWTLEPAPAPKEDALTVVGDWLGDARENDVFEHAGARDVIRREDFAKTGATTMREVLNRIPGVSAPENNGTGSHDLAMNFGIRGLNPRLASRSTVLMDGIPVPFAPYGQPQLSLAPVSLGNMDAIDVVRGGGAVRYGPQSVGGVVNFVTRAIPQDFGIEAGVEGQLSPTSSQNNPKETHNLMVGGTADNGFGTALLYSGTRGSDWREHSATRIDDLMLKSKYAPNEVHTFNSLLQYYDGEADMPGGLSRADYDADRWQSTRPYDRFWGRRKLASLGYQFQPDSQHKFNILGFYTQTLRSGYLEQGKRITLSPRNYWVRGIEPRYSQSFMIGPSAHEVGVGYRYVNESTHEMRYYTATSSGQLPSGSSPYDRDTRSGTEAHAWYLDDKIDIGNWTITPGMRFEHIESYQNNAIKGTHEEVSYNAPLPALNVLYHLTDSWNLYANTEGSFGTVQYSQIGKAVQSGNVEPEKARTWELGTRYDDGALTAEMGLFLINFNNQYDSNQTNDTVTARGKTRHTGLETQARYDLGTLTPTLDNVSVYASYAYVNAEIREKGDTYGNQVPFSPKHKGTLGVDYKPGNWTFNLNSDFQSSQFADNANTVKESADGSTGRIPGFMLWGARVAYDFGPQMADLNLAFGVKNIFDQDYFIRSYDDNNKGIYAGQPRTLYMQGSLKF

SEQ ID NO: 43 - DNA, 2022 bp, Escherichia coli
  source 1..2022: mol_type = genomic DNA
atgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcatctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggatgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttctga

SEQ ID NO: 44 - protein, 651 aa, Escherichia coli
QTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 45 - protein, 673 aa, Escherichia coli
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 46 - protein, 12 aa, synthetic construct
MRGSHHHHHHGS

SEQ ID NO: 47 - protein, 22 aa, synthetic construct
MRGSHHHHHHGSGSGSGIEGRP

(19) United States
(12) Patent Application Publication
(10) Pub. No.: US 2025/0011375 A1
(43) Pub. Date: Jan. 9, 2025

(54) METHODS AND COMPOSITIONS FOR PREVENTING INFECTION

(71) Applicant: Vaxxinova US, Inc., Willmar, MN (US)

(72) Inventors: Graham J.M. Cox, Omaha, NE (US); Daryll Emery, New London, MN (US); Darren E. Straub, New London, MN (US); Lisa Herron-Olson, St. Paul, MN (US); Patricia Tam, St. Paul, MN (US); Drew M. Catron, St. Paul, MN (US)

(21) Appl. No.: 18/705,332

(22) PCT Filed: Oct. 28, 2022
(86) PCT No.: PCT/US2022/048262

§ 371 Date: Apr. 26, 2024

Related U.S. Application Data
(60) Provisional application No. 63/273,330, filed on Oct. 29, 2021.

(51) Int. Cl. C07K 14/245; A61K 39/00; A61K 39/108; A61P 31/04; C07K 16/12; C12N 15/70

(52) U.S. Cl. CPC C07K 14/245; A61K 39/0258; A61P 31/04; C07K 16/1232; C12N 15/70; A61K 2039/505

(57) ABSTRACT

The present invention provides isolated proteins isolatable from an E. coli, such as avian pathogenic E. coli. Also provided by the present invention are compositions that include one or more of the proteins, and methods for making and methods for using the proteins.

### CROSS-REFERENCE TO RELATED APPLICATIONS

This application claims the benefit of U.S. Provisional Application Ser. No. 63/273,330, filed Oct. 29, 2021, which is incorporated by reference herein in its entirety

### SEQUENCE LISTING

This application contains a Sequence Listing electronically submitted via EFS-Web to the United States Patent and Trademark Office as an ASCII text file entitled “0293.000061WO01.xml” having a size of 92 kilobytes and created on Oct. 28, 2022.

The information contained in the Sequence Listing is incorporated by reference herein.

### BACKGROUND

E. coli is a gram-negative bacillus belonging to the family of Enterobacteriaceae. E. coli is considered a member of the normal microflora of the poultry intestine, but certain strains, such as those designated as avian pathogenic E. coli (APEC), can spread into various internal organs and cause colibacillosis. Colibacillosis is a major cause of morbidity and death of birds in the poultry industry worldwide and outbreaks have been reported in ducks, chickens, and turkeys resulting in heavy economic losses. The disease causes a variety of clinical manifestations such as airsacculitis, pericarditis, perihepatitis and peritonitis but can also be associated with yolk sac infection, omphalitis, respiratory tract infection, swollen head syndrome, septicemia, polyserositis, coligranuloma, enteritis, cellulitis, and salpingitis (Kathayat et al., Pathogens. 2021 Apr. 12; 10(4):467; Kabir, Int. J. Environ. Res. Public Health 2010, 7(1), 89-114).

In commercial chicken breeder, layer and broilers, APEC is responsible for salpingitis/peritonitis/salpingoperitonitis (SPS) leading to economic losses mainly due to mortality and loss of egg production. Birds with salpingitis show an inflamed oviduct that is frequently distended, thin-walled and filled with caseous exudate. The spread of E. coli into the abdominal cavity through the compromised oviduct wall results in concurrent peritonitis (salpingoperitonitis) with accumulation of caseating exudate in the abdominal cavity. This exudate often has the appearance of coagulated yolk, commonly identified as “egg peritonitis”. Peritonitis in the absence of salpingitis can also occur, but is uncommon (Jordan et al., Vet Rec. 2005; 157:573-577; Landman and Cornelissen, Tijdschrift Voor Diergeneeskunde, 1 Nov. 2006, 131(22):814-822; Barnes et al., Colibacillosis. In Y. M. Saif, A. M. Fadly, J. R. Glisson, L. R. McDougald, L. K. Nolan, & D. E. Swayne (Eds.). Diseases of Poultry 2008, (12 ed., pp. 691732). Ames: Iowa State Press). Internal laying may accompany salpingitis and as a consequence free yolk may be present in the abdominal cavity, which may favor the occurrence of peritonitis (Gross and Siegel, Avian Diseases. Vol. 3, No. 4 (1959), pp. 370-373).

It has been well documented that APEC strains can colonize the gastrointestinal and respiratory tracts of chickens, turkeys and ducks without causing disease and translocate to extra-intestinal sites in the presence of stressors (production-related stress, immunosuppression, and concurrent infections) as an opportunistic pathogen. APEC invades the gastrointestinal and respiratory tracts through abraded tracheal and intestinal epithelium in the presence of stressors and reaches bloodstream and internal organs resulting in a variety of clinical manifestations (Dziva and Stevens, 2008, Avian Pathol. 37:355-366; Collingwood et al., 2014, Front. Vet. Sci. 1:5). With peritonitis the natural route of infection with E. coli appears to be largely by the ascending movement of the bacteria via the cloaca to the oviduct and into the peritoneal cavity, but intratracheal introduction and infection may also occur (Landman and van Eck, Avian Pathology, 2015, 44:5, 370-378; Nolan et al., Colibacillosis. 2013, Diseases of Poultry 13 ed, Pages 751-805; Landman et al., 2013. Avian Pathology, 42(2):157-162). The route of infection is likely influenced by the type of housing insofar as proximity to litter, severity of dust and environmental exposure. It is thought that the former route may be due to the relaxation of the inner circular vaginal musculature as a consequence of intensive egg production (Nolan et al., Colibacillosis. 2013, Diseases of Poultry 13 ed, Pages 751-805; Landman et al., 2013. Avian Pathology, 42(2): 157-162). This is consistent with general industry observations that large surges in mortality and peritonitis occur around peak egg production in broiler breeder and egg-producing stock. Most APEC isolates are of three dominant serotypes: O1a:K1; O2a:K1; and O78:K80. Other serological groups less frequently isolated in disease outbreaks are O3, O6, O8, O11, O15, O22, O55, O74, O88, O95, and O109 (Zanella et al., 2000. Avian Pathology, 29:4, 311-317; Huja et al., mBio. 2015 Jan. 13; 6(1):e01681-14). The decrease in antibiotic use and the increase in aviary and free-range housing are believed to be significant contributors to the increased incidence of APEC-related mortality and peritonitis observed in recent years.

### SUMMARY OF THE APPLICATION

The present disclosure is directed to compositions which are effective to immunize domestic fowl such as turkeys, chickens, and ducks against clinical manifestations of E. coli, including the control of peritonitis, colibacillosis, and septicemia. The disclosure also describes the successful development of a peritonitis chicken model (Example 12) and its use in demonstrating that a composition that includes metal regulated receptor proteins for divalent metal ions can prevent mortality and colonization of young and mature chickens as well as the pathology characteristic of peritonitis and colibacillosis in domesticated fowl including, but not limited to, layer chickens.

Terms used herein will be understood to take on their ordinary meaning in the relevant art unless specified otherwise. Several terms used herein and their meanings are set forth below.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing each thymidine nucleotide with a uridine nucleotide.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. As used herein, “consisting essentially of,” as it relates to a composition, product, method, or the like, means that the components of the composition, product, method, or the like are limited to the enumerated components and any other components that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Conditions that are “suitable” for an event to occur, such as for stimulating an immune response to an antigen, or “suitable” conditions are conditions that do not prevent such events from occurring. Thus, these conditions permit, enhance, facilitate, and/or are conducive to the event.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows electrophoretic profiles of Avian E. coli isolates 1966 (Lane 1) and 1967 (Lane 2) showing the metal regulated proteins expressed under iron-deplete growth conditions. The identification of each protein as examined by MALDI-TOF-MS is shown. The following proteins were identified: lane 1 includes FepA, IroN, IreA and ChuA; lane 2 includes FecA, FepA and IutA. Lanes 1 and 2 includes two other putative outer membrane proteins, OmpC and OmpA.

FIG. 2 shows summary of total mortality between vaccinated and non-vaccinated controls in three separate studies with age and different time intervals of vaccination-challenge as study parameters.

FIG. 3 shows a summary of the difference in the colonization or prevalence of E. coli in organs between vaccinated and non-vaccinated control birds with a prime-boost vaccination strategy at 28 weeks of age (WOA), 31 WOA, and challenged at 33 WOA.

FIG. 4 shows a summary of the difference in the colonization or prevalence of E. coli in organs between vaccinated and non-vaccinated control birds with a prime-boost vaccination strategy at 10 weeks of age (WOA), 18 WOA, and challenged at 20 WOA.

FIG. 5 shows a summary of Study 3 of the difference in the colonization or prevalence of E. coli in organs between vaccinated and non-vaccinated control birds with a prime-boost vaccination strategy at 4 weeks of age (WOA) and 6 WOA and challenged at 8 WOA.

FIG. 6 shows serological response to vaccination for Study 4. The geometric mean ELISA titer for each vaccination group is displayed on the Y-axis with 95% confidence intervals (calculated on log-transformed values). The ages (in weeks) at vaccinations 1 and 2 are indicated. Week 0 vaccinations in the figure are one day-of-age vaccination. “None” means no vaccination of chickens in that group were done at that time. All chickens were bled for serology at 18 weeks of age. Details of the groups can also be found in Table 4. *indicates AlOH adjuvant was used in place of the water-in-oil adjuvant. Group 1, age at vaccination 1 was one day-of-age with AlOH adjuvant (0*) and age at vaccination 2 was 16 weeks (16); Group 2, age at vaccination 1 was one day-of-age with AlOH adjuvant (0*) and no second vaccination (None); Group 3, age at vaccination 1 was one day-of-age (0) and age at vaccination 2 was 16 weeks (16); Group 4, age at vaccination 1 was 12 weeks (12) and age at vaccination 2 was 16 weeks (16); Group 5, no first vaccination (None) and age at vaccination 2 was 16 weeks (16); Group 6, no first vaccination (None) and no second vaccination (None).

FIG. 7 shows the percent mortality of mice vaccinated with individual recombinant proteins compared to non-vaccinated controls following challenge. Mice received two vaccinations three weeks apart and challenged 3 weeks post second vaccination.

FIG. 8 shows the percent mortality of mice vaccinated with multiple recombinant proteins in single vaccine formulations compared to non-vaccinated controls following challenge. Formulations evaluated were the combination of IreA and ChuA, the combination of FepA and IroN, the combination of ChuA and IroN, the combination of IreA, ChuA, FepA, and IroN, and the combination of IreA, ChuA, FyuA, and IroN.

FIG. 9 shows examples of amino acid sequences of proteins described herein and examples of nucleotide sequences encoding the proteins.

### DETAILED DESCRIPTION

### Proteins

In one aspect, this disclosure provides proteins, compositions including proteins, and methods of making and using the proteins. As used herein, “protein” refers to a polymer of amino acids linked by peptide bonds. Thus, for example, the terms peptide, oligopeptide, polypeptide, and enzyme are included within the definition of protein. This term also includes proteins that may include one or more post-expression modifications of the protein such as, for example, a glycosylation, an acetylation, a phosphorylation, and the like. The term protein does not connote a specific length of a polymer of amino acids. A protein may be isolatable directly from a natural source or can be prepared with the aid of recombinant, enzymatic, or chemical techniques. In the case of a protein that is naturally occurring, such a protein is typically isolated.

An “isolated” protein is one that has been removed from its natural environment. For instance, an isolated protein is a protein that has been removed from the cytoplasm or from the membrane of a cell, and many of the proteins, nucleic acids, and other cellular material of its natural environment are no longer present. A mixture of more than one protein, each of which have been removed from their natural environments, are considered isolated.

A protein characterized as “isolatable” from a particular source is a protein that, under appropriate conditions, is produced by the identified source, although the protein may be obtained from alternate sources using, for example, conventional recombinant, chemical, or enzymatic techniques. Thus, characterizing a protein as “isolatable” from a particular source does not imply any specific source from which the protein must be obtained or any particular conditions or processes under which the protein must be obtained.

A “purified” protein is one that is at least 60% free, preferably at least 75% free, and most preferably at least 90% free from other components with which they are naturally associated. Proteins that are produced outside the organism in which they naturally occur, e.g., through chemical or recombinant means, are considered to be isolated and purified by definition, since they were never present in a natural environment.

Generally, a protein may be characterized by molecular weight, amino acid sequence, nucleic acid sequences that encodes the protein, immunological activity, or any combination of two or more such characteristics. The molecular weight of a protein, typically expressed in kilodaltons (kDa), can be determined using routine methods including, for instance, gel filtration, gel electrophoresis including sodium dodecyl sulfate (SDS) polyacrylamide gel electrophoresis (PAGE), capillary electrophoresis, mass spectrometry, liquid chromatography (including HPLC), and calculating the molecular weight from an observed or predicted amino acid sequence. Unless indicated otherwise, reference to molecular weight refers to molecular weight as determined by resolving a protein using an SDS polyacrylamide gel having a stacking gel of about 4% and a resolving gel of about 10% under reducing and denaturing conditions. A molecular weight of a protein determined by SDS-PAGE is also referred to herein as an apparent molecular weight. In one embodiment, the molecular weight of a protein identified by SDS-PAGE includes molecular weights of 1, 2, 3, 4, or 5 kDa above and below the stated value.

The proteins described herein may be metal-regulated. As used herein, a “metal-regulated protein” is a protein that is expressed by a microbe at a greater level when the microbe is grown in low metal conditions compared to when the same microbe is grown in high metal conditions. Low metal and high metal conditions are described herein. For instance, certain metal-regulated proteins produced by E. coli are not expressed at detectable levels during growth of the microbe in high metal conditions but are expressed at detectable levels during growth in low metal conditions. Other proteins described herein are expressed at detectable levels during growth of the microbe in high metal conditions but more of the protein is expressed during growth in low metal conditions. The expression of such proteins is referred to herein as “enhanced” during growth in low metal conditions. Typically, the increase in expression of a protein during growth in low metal conditions is between 20% and 500% compared to the expression of the protein during growth in high metal conditions.

As described herein (see Example 1), 440 field isolates of E. coli were obtained from infected organs (liver, spleen and/or oviducts) of chickens and turkeys showing clinical signs of peritonitis or related clinical signs of colibacillosis. Each E. coli isolate was grown in iron replete and iron deplete media conditions and protein-enriched extracts were derived from each isolate. After size-fractionation by SDS-PAGE, visual comparison of the SDS protein banding profiles showed that a few protein banding profile types (that is, a combination of iron-regulated proteins expressed by a single E. coli) existed across this diverse population. Two isolates were chosen (isolate APEC-1966 and isolate APEC-1967) on the basis of their protein banding profile which, together, represented the banding patterns that contained many of the proteins in those isolates that induced peritonitis. The inventors hypothesized that a composition that included the iron regulated proteins expressed by these two strains would provide protection against peritonitis caused by a broad variety of E. coli typically observed in an outbreak in the field.

Mass spectrometry analyses of trypsin fragments of gel-isolated iron-regulated proteins from isolates APEC-1966 and APEC-1967 and identification of open reading frames identified the following iron-regulated proteins: ChuA, IroN, IreA, IutA, FecA and FepA. Genomic analyses by PCR of the isolates APEC-1966 and APEC-1967 indicated that genes for other iron regulated proteins (FhuE, Fiu, CirA FhuA, FyuA and BtuB) exist in these isolates, but it is unknown if they are expressed during iron-restricted antigen production.

Table 1 summarizes the expression of proteins identified in the isolates APEC-1966 and APEC-1967 during growth in the absence of iron.

TABLE 1

| | | Proteins expressed by E. coli Isolates | | | | | | | |
|---|---|---|---|---|---|---|---|---|---|
| Protein Analysis | Isolate ID | 1966 and 1967 as Examined by MALDI-TOF | | | | | | | |
| MALDI-TOF | 1966 | | FepA | | IroN | IreA | ChuA | OmpC | OmpA |
| Predicted MW (kDa) | | | 82 | | 79 | 75 | 73 | 41 | 37 |
| SDS-PAGE MW | | | 83 | | 78 | 74 | 70 | 36 | 33 |
| (kDa) | | | | | | | | | |
| Example of protein | | | 8 | | 6 | 4 | 2 | — | — |
| (SEQ ID NO:) | | | | | | | | | |
| MALDI-TOF | 1967 | FecA | FepA | IutA | | | | | |
| Predicted MW (kDa) | | 85 | 82 | 81 | | | | | |
| SDS-PAGE MW | | 82 | 80 | 76 | | | | 35 | 34 |
| (kDa) | | | | | | | | | |
| Example of protein | | 10 | 8 | 12 | | | | | |
| (SEQ ID NO:) | | | | | | | | | |

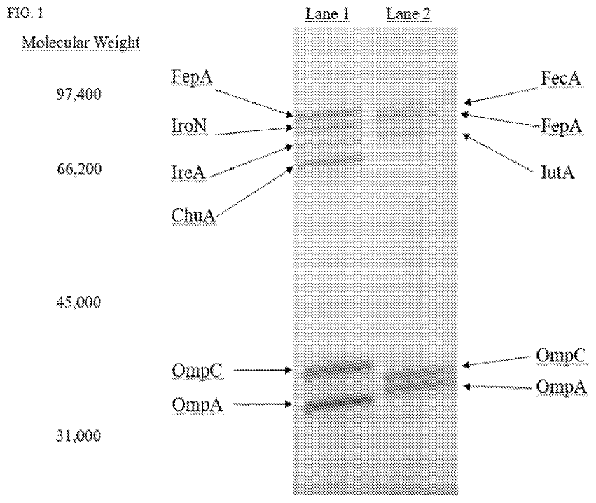

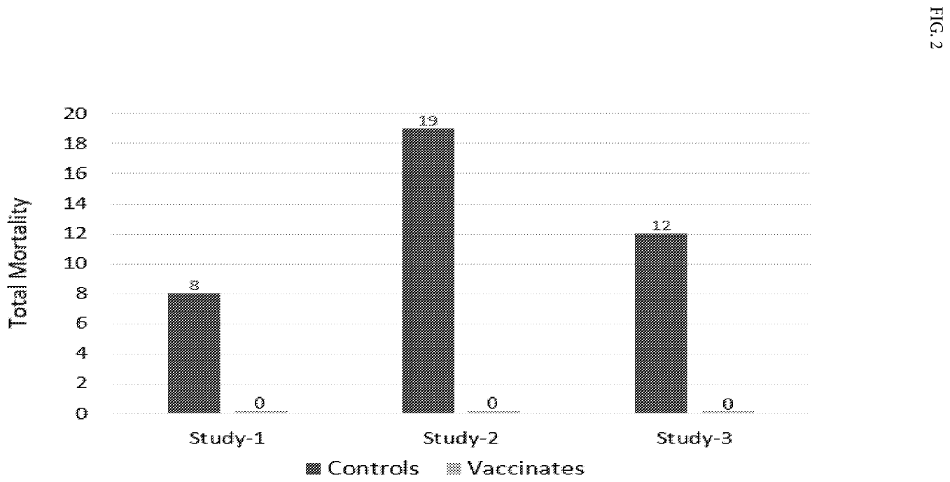

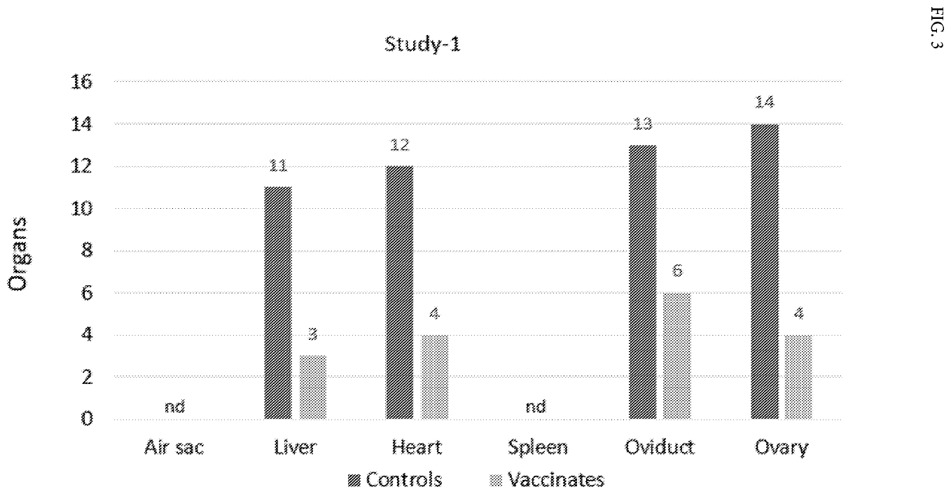

Table 1. Protein Analysis: SDS-PAGE bands were excised from the acrylamide gel (FIG. 1) and examined by MALDI-TOF-MS to determine the identity of individual proteins (see Example 2). MW, molecular weight. The Predicted MW of each protein was based the amino acid sequence derived from the genome of each isolate. The SDS-PAGE MW of each protein was determined by resolving proteins on a SDS polyacrylamide gel.

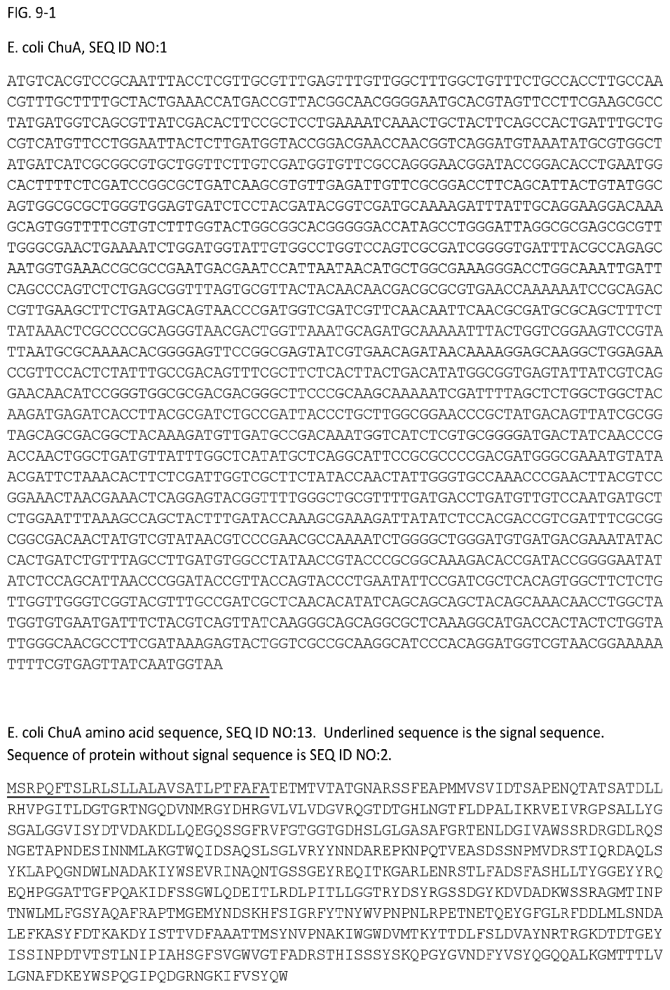

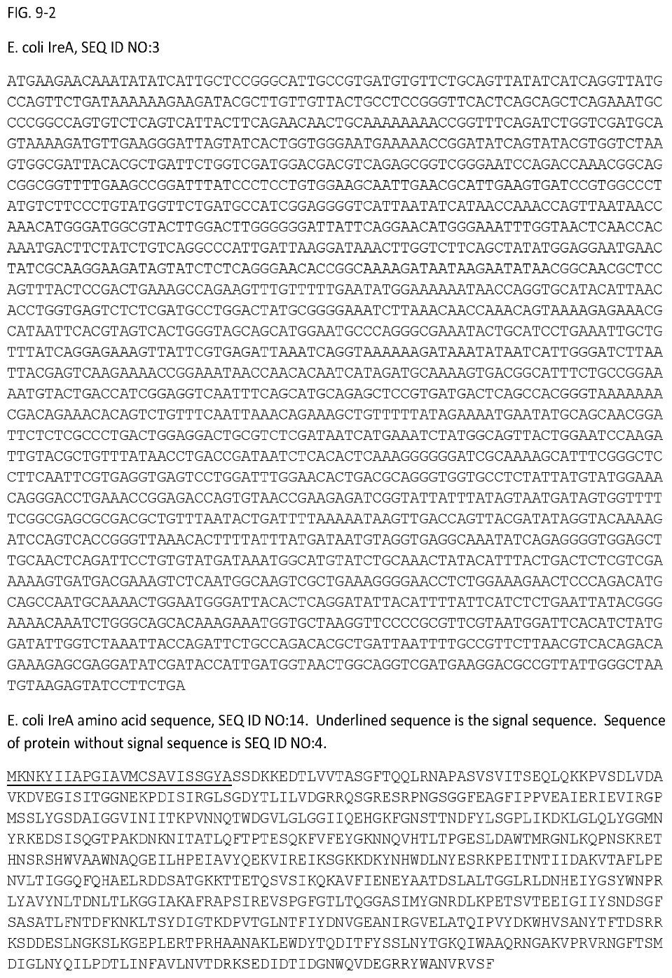

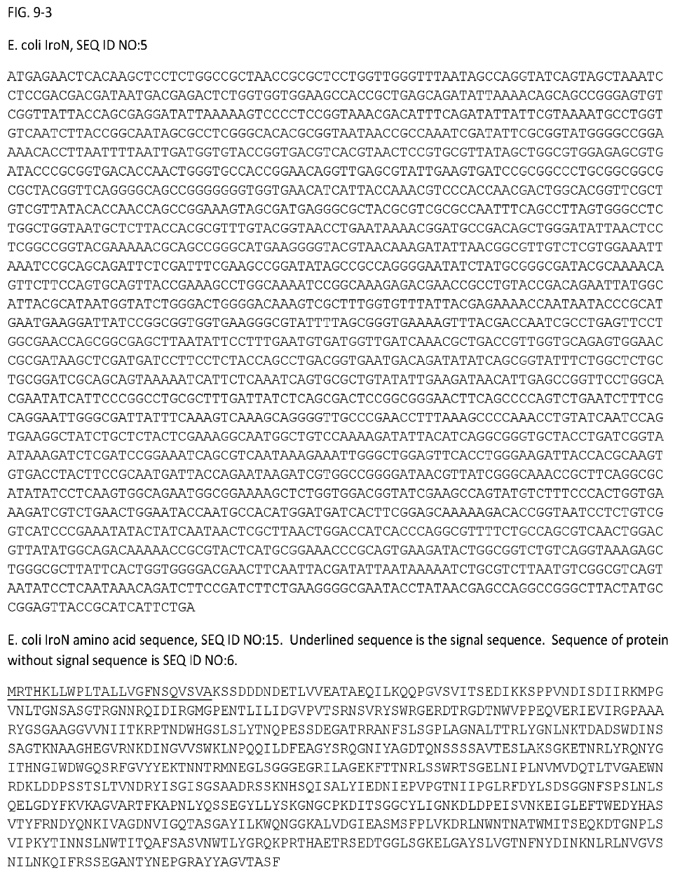

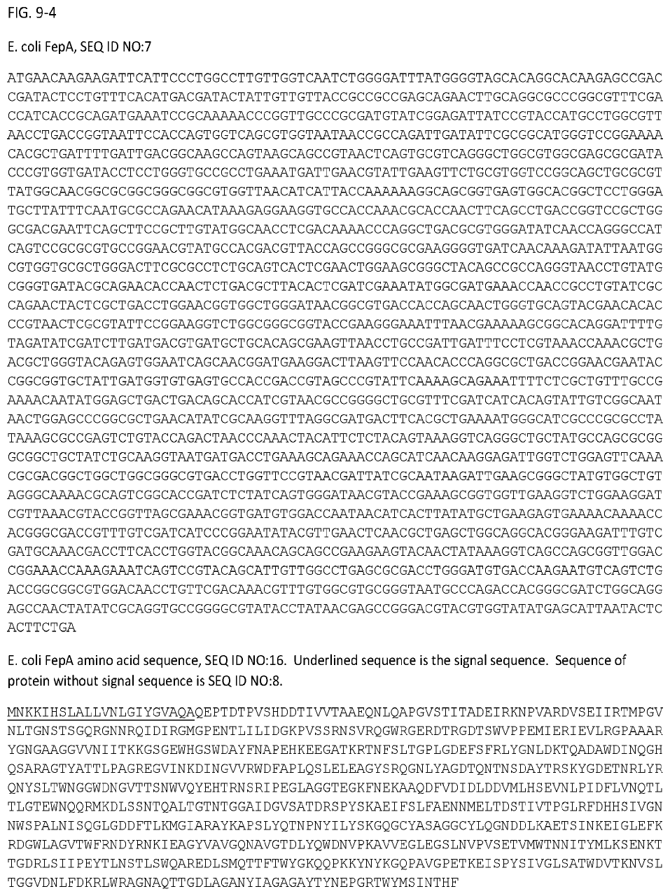

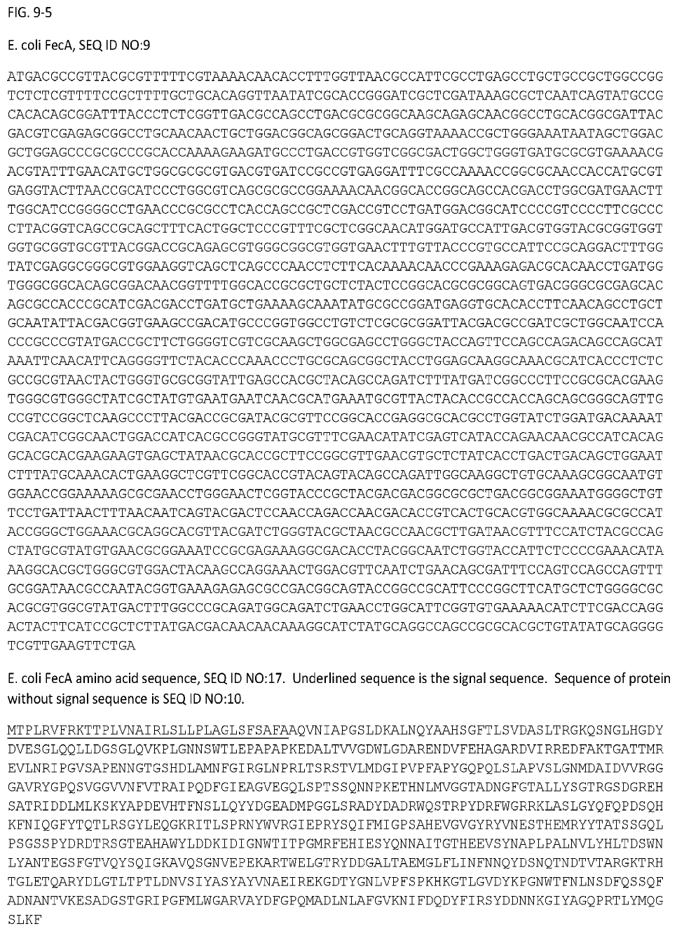

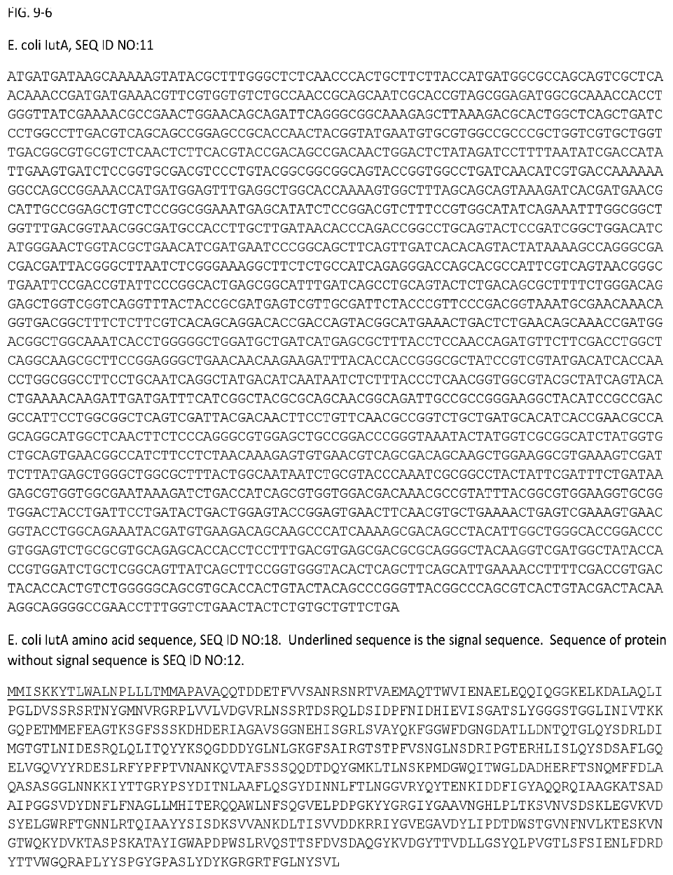

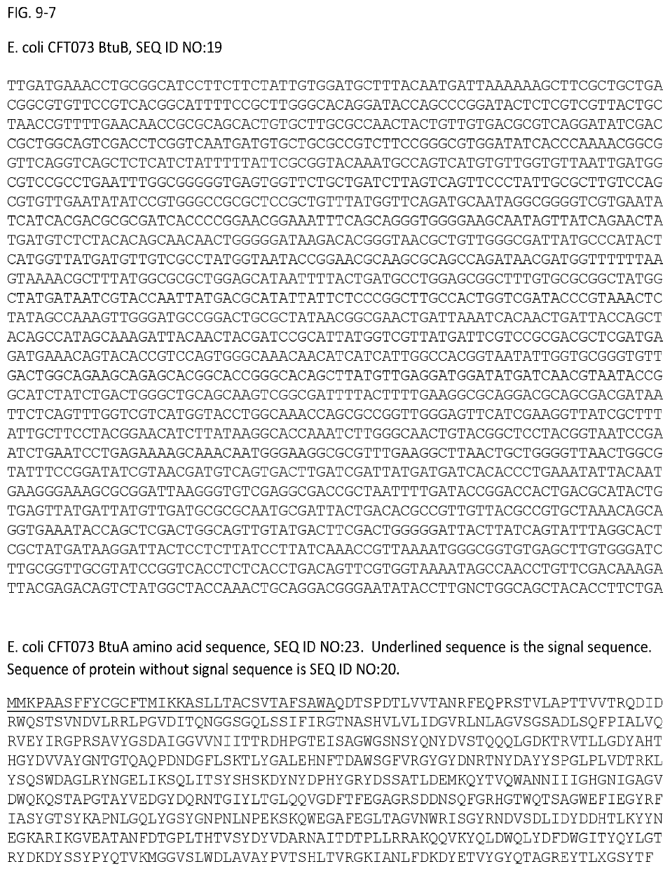

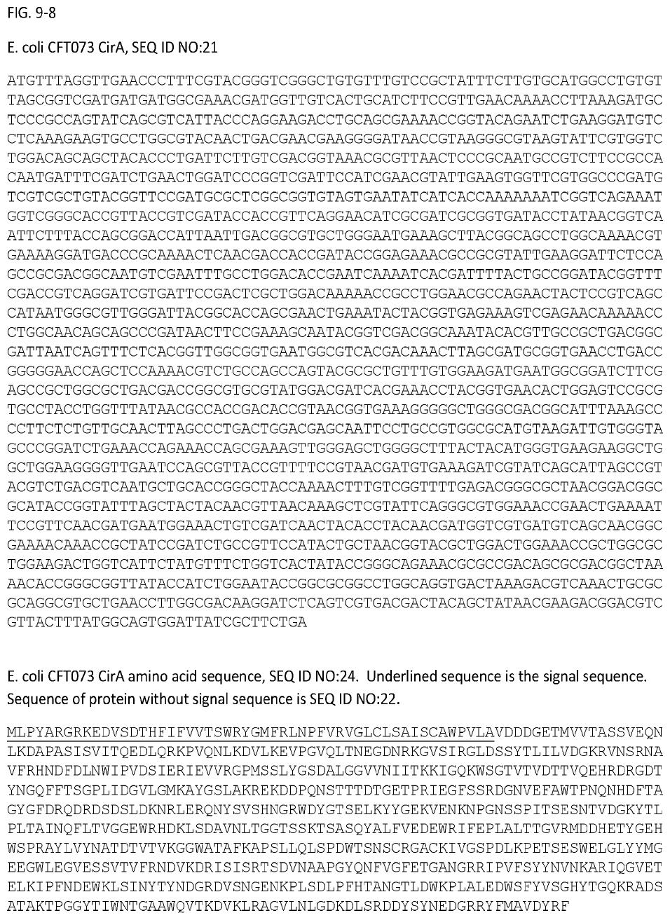

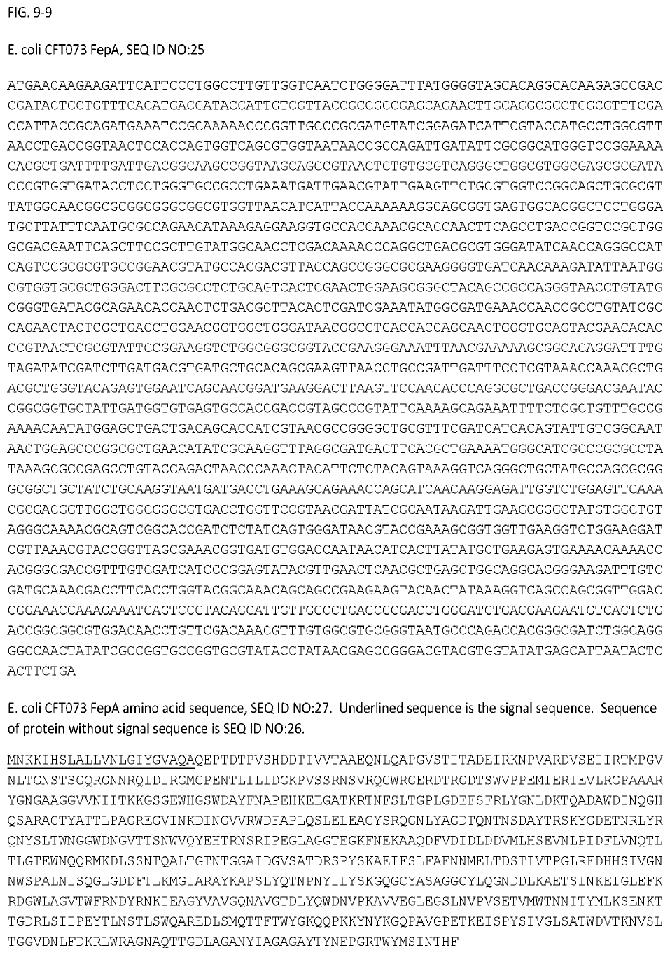

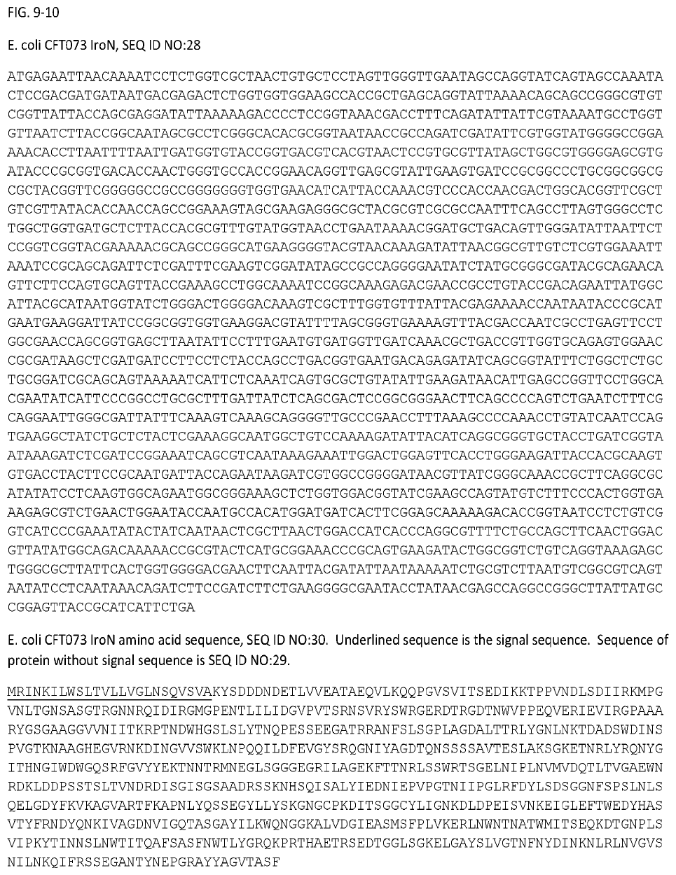

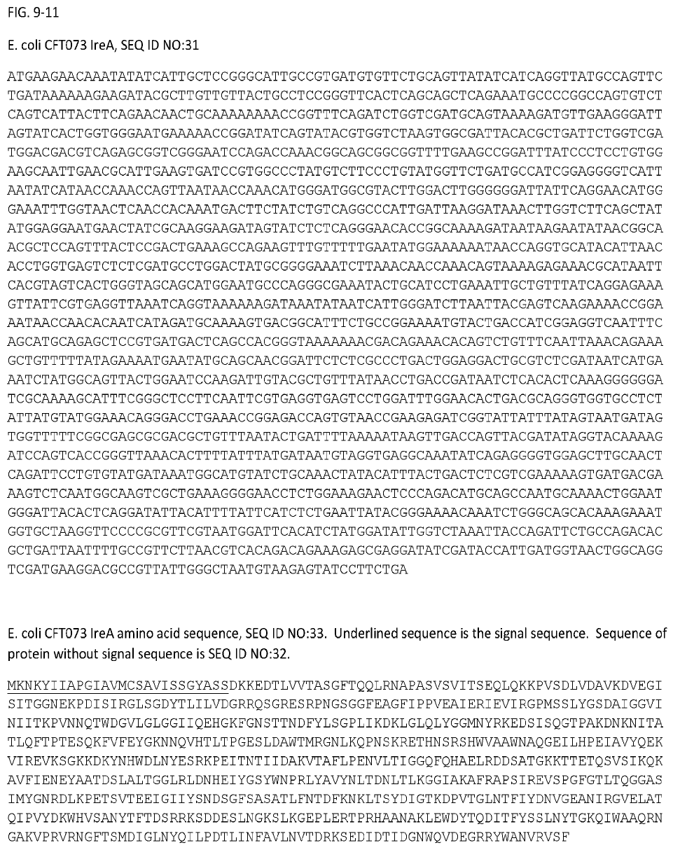

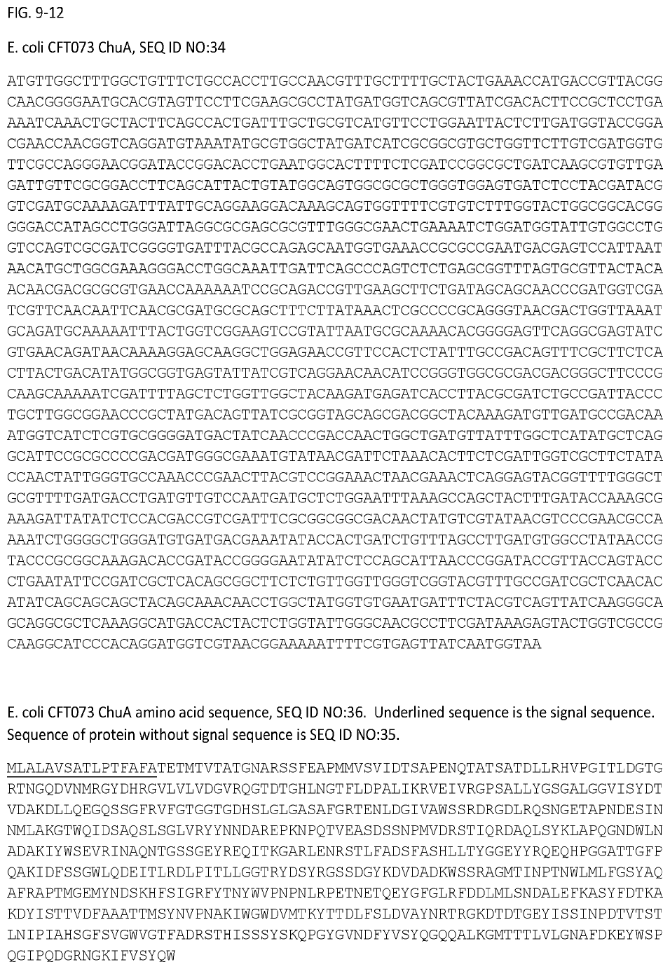

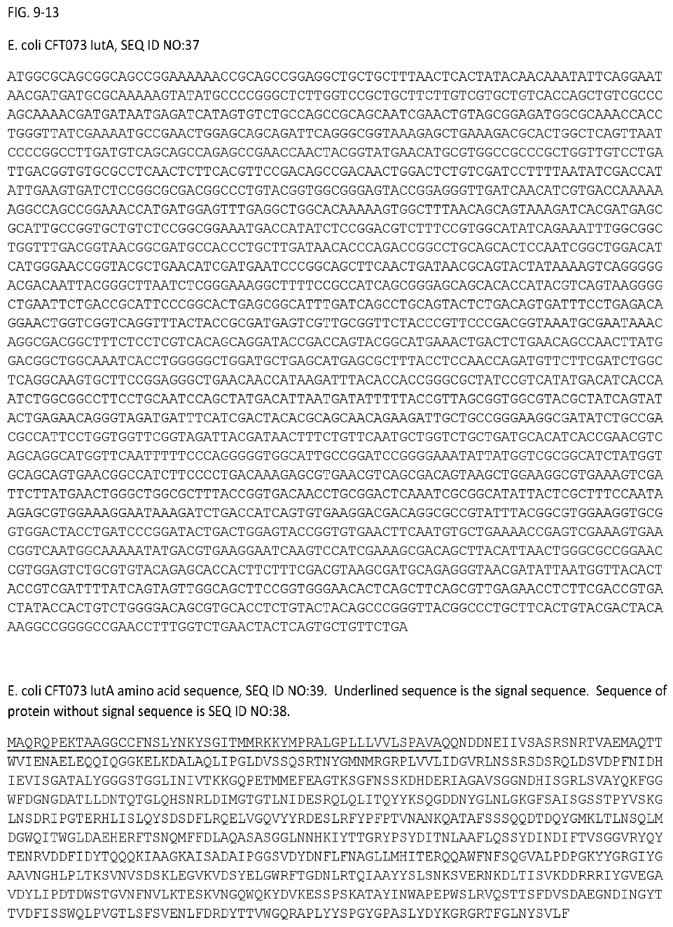

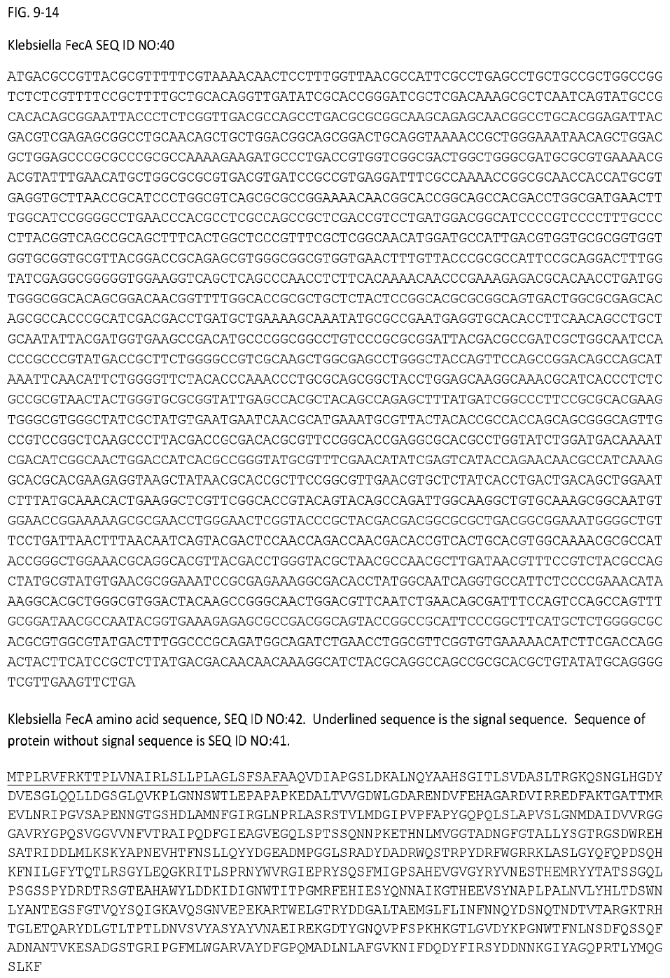

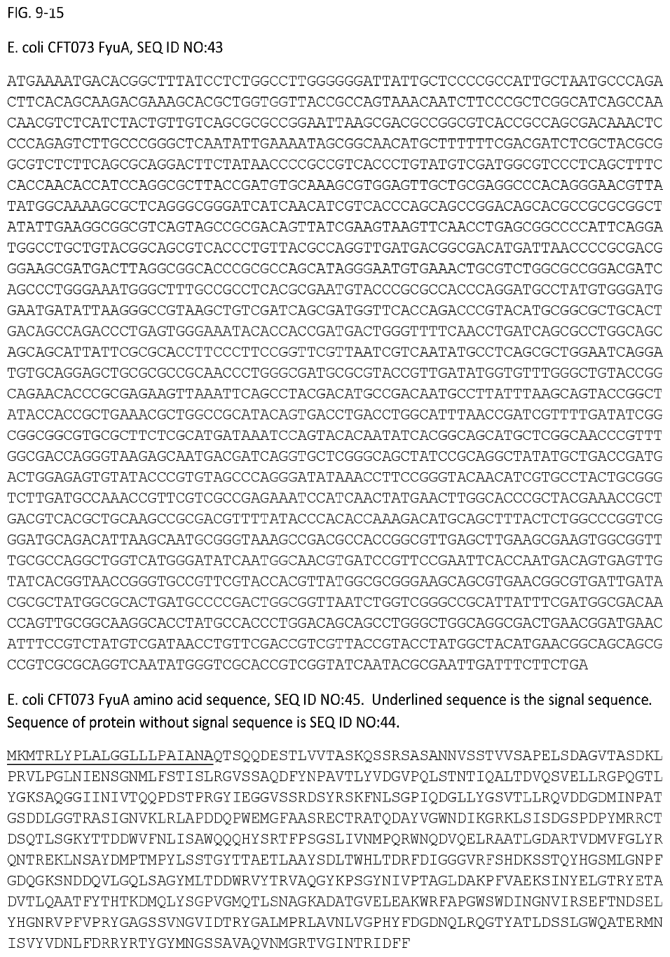

Proteins provided by the present disclosure include those shown at SEQ ID NO:2, 4, 6, 8, 10, 12, 13, 14, 15, 16, 17, 18, 20, 22, 23, 24, 44, and 45 (FIG. 9). Also provided are proteins that are structurally similar to a protein of SEQ ID NO:2, 4, 6, 8, 10, 12, 13, 14, 15, 16, 17, 18, 20, 22, 23, 24, 44, or 45. As used herein, a protein may be “structurally similar” to a reference protein if the amino acid sequence of the protein possesses a specified amount of sequence similarity and/or sequence identity compared to the reference protein. Thus, a protein may be “structurally similar” to a reference protein if, compared to the reference protein, it possesses a sufficient level of amino acid sequence identity, amino acid sequence similarity, or a combination thereof. In one embodiment, such a protein is metal-regulated when expressed by an E. coli.

Structural similarity of two proteins can be determined by aligning the residues of the two proteins (for example, a candidate protein and any appropriate reference protein described herein, e.g., SEQ ID NO:2, 4, 6, 8, 10, 12, 13, 14, 15, 16, 17, 18, 20, 22, 23, 24, 44, or 45) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein or any known metal-regulated protein, as appropriate. A candidate protein is the protein being compared to the reference protein. A candidate protein can be isolated, for example, from a microbe, or can be produced using recombinant techniques, or chemically or enzymatically synthesized.

Unless modified as otherwise described herein, a pair-wise comparison analysis of amino acid sequences can be carried out using the BESTFIT algorithm in the GCG package (version 10.2, Madison WI). Alternatively, proteins may be compared using the Blastp program of the BLAST 2 search algorithm, as described by Tatiana et al. (FEMSMicrobiol Lett, 174:247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a protein containing deletions or additions of one or more contiguous or noncontiguous amino acids that do not eliminate a functional activity-such as, for example, immunological activity—of the protein are also contemplated.

Thus, as used herein, reference to a protein as described herein and/or reference to the amino acid sequence of one or more SEQ ID NOs can include a protein with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to a protein as described herein and/or reference to the amino acid sequence of one or more SEQ ID NOs can include a protein with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Unless a specific level of sequence similarity and/or identity is expressly indicated herein (e.g., at least 80% sequence similarity, at least 90% sequence identity, etc.), reference to the amino acid sequence of an identified SEQ ID NO includes variants having the levels of sequence similarity and/or the levels of sequence identity described herein.

The skilled person will recognize that the ChuA protein depicted at SEQ ID NO:2 and 13 can be compared to ChuA proteins from other E. coli using readily available algorithms such as ClustalW to identify regions of conservation and variability of ChuA proteins. Using this information, the skilled person will be able to readily predict with a reasonable expectation that certain conservative substitutions to a protein such as SEQ ID NO:2 will not decrease activity of the protein. The same is true for the IreA protein depicted at SEQ ID NO:4 and 14, the IroN protein depicted at SEQ ID NO:6 and 15, the FepA protein depicted at SEQ ID NO:8 and 16, the FecA protein depicted at SEQ ID NO:10 and 17, the IutA protein depicted at SEQ ID NO:12 and 18, the BtuB protein depicted at SEQ ID NO:20 and 23, the CirA protein depicted at SEQ ID NO:22 and 24, and the FyuA protein depicted at SEQ ID NO:44 and 45. A person of ordinary skill in the art can deduce from such alignments regions of a protein in which substitutions, particularly conservative substitutions, may be permitted without unduly affecting biological activity of the modified protein. Consequently, a protein as described herein can include certain variants including, for example, homologous proteins that originate-biologically and/or recombinantly—from microbial species or strains other than the microbial species or strain from which the protein was originally isolated and/or identified.

Other examples of E. coli ChuA proteins include the proteins at Genbank accession numbers EF05360954.1, WP_021522278.1, and WP_054494517.1. Other examples of E. coli IreA proteins include the proteins at Genbank accession numbers WP_115722697.1, WP_000792545.1, and WP_061362196.1. Other examples of E. coli IroN proteins include the proteins at Genbank accession numbers WP_097697489.1, WP_001523424.1, and WP_125108203.1. Other examples of E. coli FepA proteins include the proteins at Genbank accession numbers WP_001034893.1, WP_021512183.1, and WP_032260138.1. Other examples of E. coli FecA proteins include the proteins at Genbank accession numbers WP_089602808.1, WP_119122509.1, and M1BB7137362.1. Other examples of E. coli IutA proteins include the proteins at Genbank accession numbers WP_001553716.1, WP_039025836.1, and WP_098722385.1.

A protein as described herein also can be designed to provide one or more additional sequences such as, for example, the addition of coding sequences for added C-terminal and/or N-terminal amino acids that may facilitate purification by trapping on columns or use of antibodies. Such tags include, for example, histidine-rich tags that allow purification of proteins on nickel columns. Such gene modification techniques and suitable additional sequences are well known in the molecular biology arts. A protein as described herein also may be designed so that certain amino acids at the C-terminal and/or N-terminal are deleted.

A “modification” of a protein as described herein includes a protein (or an analog thereof such as, e.g., a fragment thereof) that is chemically or enzymatically derivatized at one or more constituent amino acids. Such a modification can include, for example, a side chain modification, a backbone modification, an N-terminal modification, and/or a C-terminal modification such as, for example, acetylation, hydroxylation, methylation, amidation, and the attachment of a carbohydrate and/or lipid moiety, a cofactor, and the like, and combinations thereof. Modified proteins as described herein may retain the biological activity-such as, for example, immunological activity—of the unmodified protein or may exhibit a reduced or increased biological activity compared to the unmodified protein.

The proteins described herein may have immunological activity. “Immunological activity” refers to the ability of a protein to elicit an immunological response in an animal. An immunological response to a protein is the development in an animal of a cellular and/or antibody-mediated immune response to the protein. Usually, an immunological response includes but is not limited to one or more of the following effects: the production of antibodies, B cells, helper T cells, suppressor T cells, and/or cytotoxic T cells, directed to an epitope or epitopes of the protein. “Epitope” refers to the site on an antigen to which specific B cells and/or T cells respond so that antibody is produced. The immunological activity may be protective. “Protective immunological activity” refers to the ability of a protein to elicit an immunological response in an animal that inhibits or limits infection by E. coli. Whether a protein has protective immunological activity can be determined by methods known in the art such as, for example, methods described in Example 17. A protein may have seroactive activity. As used herein, “seroactive activity” refers to the ability of a candidate protein to react with antibody present in convalescent serum from an animal infected with an E. coli.

A protein as described herein (including a biologically active analog thereof and/or a modification thereof) can include a native (naturally occurring), a recombinant, a chemically synthesized, or an enzymatically synthesized protein. For example, a protein as described herein may be prepared by isolating the protein from a natural source, e.g., an E. coli that naturally expresses one or more of the proteins described herein or may be prepared recombinantly by conventional methods including, for example, preparation as fusion proteins in bacteria or other host cells.

A protein expressed by a reference microbe can be obtained by growing the reference microbe under low metal conditions as described herein and the subsequent isolation of a protein by the processes disclosed herein. Alternatively, a protein expressed by a reference microbe can be obtained by identifying coding regions expressed at higher levels when the microbe is grown in low metal conditions—i.e., metal-regulated. A metal-regulated coding region can be cloned and expressed, and the expressed metal-regulated protein may be identified by the processes described herein. A candidate protein can be isolatable from an E. coli.

The present disclosure also provides proteins that are metal-regulated and isolatable from an E. coli after growth in low iron conditions. Each protein can be expressed by a microbe at a greater level when the microbe is grown in low metal conditions compared to when the same microbe is grown in high metal conditions. Certain metal-regulated proteins produced by E. coli are not expressed at detectable levels during growth of the microbe in high metal conditions but are expressed at detectable levels during growth in low metal conditions. Other proteins described herein are “enhanced” during growth in low metal conditions; they are expressed at detectable levels during growth of the microbe in high metal conditions but more of the protein is expressed during growth in low metal conditions.

A metal-regulated protein that is isolatable from an E coli after growth in low iron conditions can have a molecular weight of 82 kDa, 80-83 kDa, 78 kDa, 76 kDa, 74 kDa, or 70 kDa as determined by SDS-PAGE (see Table 1). In one embodiment, the low iron condition is growth in the presence of 2,2′-dipyridyl. An example of an 82 kDa metal-regulated protein isolatable from an E. coli is SEQ ID NO:10 (a FecA protein). An example of an 83 to 80 kDa metal-regulated protein isolatable from an E. coli is SEQ ID NO:8 (a FepA protein). An example of a 76 kDa metal-regulated protein isolatable from an E. coli is SEQ ID NO:12 (an IutA protein). An example of a 78 kDa metal-regulated protein isolatable from an E. coli is SEQ ID NO:6 (an IroN protein). An example of a 74 kDa metal-regulated protein isolatable from an E. coli is SEQ ID NO:4 (an IreA protein). An example of a 70 kDa metal-regulated protein isolatable from an E. coli is SEQ ID NO:2 (a ChuA protein). Reference to molecular weight here refers to molecular weight as determined by resolving a protein using an SDS polyacrylamide gel having a stacking gel of about 4% and a resolving gel of about 10% under reducing and denaturing conditions. The skilled person will recognize that determining molecular weight by SDS-PAGE electrophoresis is less accurate than calculating the molecular weight of a protein based on its amino acid sequence, and as a result the molecular weight of a protein identified by SDS-PAGE includes a range of molecular weights of 1, 2, 3, 4, or 5 kDa above and below the stated value.

This disclosure also describes certain proteins that are not metal-regulated. Such proteins are expressed in the presence of a metal ion such as, for example, in the presence of ferric chloride, and also expressed when grown in low iron conditions. Examples of this type of protein isolatable from E. coli are shown as the lower molecular weight OmpC and OmpA proteins of FIG. 1.

### Polynucleotides

Proteins as described herein also may be identified in terms of the polynucleotide that encodes the protein (see FIG. 9). Thus, this disclosure provides polynucleotides that encode a protein as described herein or hybridize, under standard hybridization conditions, to a polynucleotide that encodes a protein as described herein, and the complements of such polynucleotide sequences.

As used herein, reference to a polynucleotide as described herein and/or reference to the nucleic acid sequence of one or more SEQ ID NOs:1, 3, 5, 7, 9, 11, 19, 21, or 43) can include polynucleotides having a sequence identity of at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an identified reference polynucleotide sequence.

In this context, “sequence identity” refers to the identity between two polynucleotide sequences. Sequence identity is generally determined by aligning the bases of the two polynucleotides (for example, aligning the nucleotide sequence of the candidate sequence and a nucleotide sequence that includes, for example, a nucleotide sequence disclosed herein, such as SEQ ID NO: 1, 3, 5, 7, 9, 11, 19, 21, or 43) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate sequence is the sequence being compared to a known sequence—e.g., a nucleotide sequence that includes a nucleotide sequence described herein, for example, SEQ ID NO: 1, 3, 5, 7, 9, 11, 19, 21, or 43. For example, two polynucleotide sequences can be compared using the Blastn program of the BLAST 2 search algorithm, as described by Tatusova et al., (FEMS Microbiol Lett., 174:247-250 (1999)), and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on.

Finally, a polynucleotide as described herein can include any polynucleotide that encodes a protein as described herein. Thus, the nucleotide sequence of the polynucleotide may be deduced from the amino acid sequence that is to be encoded by the polynucleotide.

### Whole Cells

This disclosure also provides whole cell preparations of a microbe, where the microbe expresses one or more of the proteins described herein. The cells present in a whole cell preparation may be inactivated such that the cells cannot replicate but the immunological activity of the proteins as described herein expressed by the microbe is maintained. Typically, the cells may be killed by exposure to heat or chemical agents such as glutaraldehyde, formalin, formaldehyde, and the like. In one embodiment, the whole cell is an E. coli that naturally expresses one or more of the proteins described herein. In one embodiment, an E. coli cell produces proteins that are identical to or structurally similar to SEQ ID NO:2 or 13 (a ChuA protein), SEQ ID NO:4 or 14 (an IreA protein), SEQ ID NO:6 or 15 (an IroN protein), and SEQ ID NO:8 or 16 (a FepA protein). An example of such an E. coli is isolate 1966 described herein. In one embodiment, an E. coli cell produces proteins that are identical to or structurally similar to SEQ ID NO:8 or 16 (a FepA protein), SEQ ID NO:10 or 17 (a FecA protein), and SEQ ID NO:12 or 18 (an IutA protein). An example of such an E. coli is isolate 1967 described herein. Other E. coli that express a subset of proteins disclosed herein, e.g., express proteins that are identical to or structurally similar to SEQ ID NO:8 or 16, SEQ ID NO:6 or 15, SEQ ID NO:4 or 14, and SEQ ID NO:2 or 13 under low iron conditions, or express proteins that are identical to or structurally similar to SEQ ID NO:10 or 17, SEQ ID NO:8 or 16, and SEQ ID NO:12 or 18 under low iron conditions can be identified by the skilled person by screening wild-type isolates as described herein (Examples 1 and 2). The inventors are not aware of a single E. coli that expresses all six of these proteins.

In one embodiment, a microbe is engineered to express a recombinantly produced protein that identical to or has structural similarity to one of the proteins described herein. For instance, a cell can be engineered to produce proteins that are identical to or structurally similar to SEQ ID NO:2 or 13 (a ChuA protein), SEQ ID NO:4 or 14 (an IreA protein), SEQ ID NO:6 15 (an IroN protein), SEQ ID NO:8 16 (a FepA protein), SEQ ID NO:10 or 17 (a FecA protein), SEQ ID NO:12 or 18 (an IutA protein), SEQ ID NO:20 or 23(a BtuB protein), SEQ ID NO:22 or 24 (a CirA protein), SEQ ID NO:44 or 45 (a FyuA protein) or any combination thereof. In one embodiment, the whole cell is a member of the family Enterobacteriaceae, such as the E. coli or the genus Salmonella, including S. Typhimurium.

### Compositions

A composition can include one protein described herein, at least one protein described herein, or a number of proteins described herein that is an integer greater than one (e.g., at least two, at least three, at least four, at least five, at least six, at least seven, at least eight, and so on). Non-limiting examples of compositions of proteins include, but are not limited to; one, two, three, four, five, or six proteins selected from ChuA, IreA, IroN, FepA, FecA, and IutA; one, two, three, four, five, or six proteins having structural similarity with SEQ ID NOs:2, 4, 6, 8, 10, or 12; the proteins in FIG. 1, lane 1, lane 2 and the combination of both lanes 1 and 2; one or more of the proteins shown in FIG. 7, and the compositions of FIG. 8.

A recombinantly-produced protein may be expressed from a vector that permits expression of the protein when the vector is introduced into an appropriate host cell. A host cell may be constructed to produce one or more recombinantly-produced proteins as described herein and, therefore, can include one or more vectors that include at least one polynucleotide encoding a protein described herein. Thus, each vector can include one or more polynucleotides as described herein—i.e., a polynucleotide that encodes a protein as described herein. Methods for the genetic manipulation of microbes, such as E. coli, are known and routine in the art.

Certain compositions such as, for example, those including recombinantly-produced proteins, can include a maximum number of proteins. In some embodiments, the maximum number of proteins can refer to the maximum total number of proteins. Certain compositions can include, for example, no more than 30 proteins, no more than 25 proteins, no more than 20 proteins, no more than 17 proteins, no more than 16 proteins, no more than 15 proteins, no more than 14 proteins, no more than 13 proteins, no more than 12 proteins, no more than 11 proteins, no more than 10 proteins, no more than nine proteins, no more than eight proteins, no more than seven proteins, no more than six proteins, no more than five proteins, no more than four proteins, no more than three proteins, no more than two proteins, or no more than one protein. In other embodiments, a maximum number of recombinantly-produced proteins may be specified in a similar manner. In still other embodiments, a maximum number of non-recombinantly-produced proteins may be specified in a similar manner.

In one embodiment, a composition can include proteins isolatable from one microbe when the microbe is engineered to express one or more proteins described herein. In one embodiment, a microbe is engineered to express one, two, three, four, five, or six proteins selected from ChuA, IreA, IroN, FepA, FecA, and IutA, or identical to or having structural similarity with SEQ ID NOs:2, 4, 6, 8, 10, or 12. Optionally, a microbe is engineered to express a BtuB protein, and CirA protein, and/or a FyuA protein, such as one identical to or structurally similar with SEQ ID NOs:20 (a BtuB protein) and/or SEQ ID NO:22 (a CirA protein) and/or SEQ ID NO:44 (a FyuA protein). In one embodiment, a composition can include proteins isolatable from two or more microbes. For instance, a composition can include proteins isolatable from two or more wild-type E. coli.

In certain embodiments, a composition can include a preparation of whole cells. In one embodiment, the preparation includes two or more whole cells, each of which express a subset of proteins that are identical to or structurally similar to SEQ ID NO:2 (a ChuA protein), SEQ ID NO:4 (an IreA protein), SEQ ID NO:6 (an IroN protein), SEQ ID NO:8 (a FepA protein), SEQ ID NO:10 (a FecA protein), SEQ ID NO:12 (an IutA protein). In one embodiment, an E. coli cell produces proteins that are identical to or structurally similar to SEQ ID NO:2 (a ChuA protein), SEQ ID NO:4 (an IreA protein), SEQ ID NO:6 (an IroN protein), and SEQ ID NO:8 (a FepA protein). An example of such an E. coli is isolate APEC-1966 described herein. In one embodiment, an E. coli cell produces proteins that are identical to or structurally similar to SEQ ID NO:8 (a FepA protein), SEQ ID NO:10 (a FecA protein), and SEQ ID NO:12 (an IutA protein). An example of such an E. coli is isolate APEC-1967 described herein. In some of these embodiments, the whole cell can be an E. coli, such as a wild type E. coli.

In one embodiment, the preparation is a whole cell that has been engineered to express one or more proteins identical to or having structural similarity with a protein described herein. In one embodiment, the microbe is engineered to express proteins selected from ChuA, IreA, IroN, FepA, FecA, and IutA, or identical to or structurally similar with SEQ ID NOs:2, 4, 6, 8, 10, 12. Optionally, a microbe is engineered to express a BtuB, a CirA, and/or a FyuA protein, such as a protein identical to or structurally similar with SEQ ID NOs:20 (a BtuB protein) and/or SEQ ID NO:22 (a CirA protein) and/or SEQ ID NO:44 (a FyuA protein). In one embodiment, the preparation is two or more populations of microbes where each of the populations express a subset of the proteins, e.g., the six proteins (proteins selected from ChuA, IreA, IroN, FepA, FecA, and IutA, or identical to or having structural similarity with SEQ ID NOs:2, 4, 6, 8, 10, and/or 12, and the optional SEQ ID NOs: 20, and/or 22, and/or 44), and the two or more populations when considered as a whole express the six proteins. In some embodiments, a composition can include whole cell preparations from two, three, four, five, or six strains.

In one embodiment, a composition can include proteins isolatable from one or more microbes when the microbe(s) naturally expresses the proteins during growth in low iron conditions. In one embodiment, the microbe is E. coli. In one embodiment, the composition includes metal-regulated proteins having a molecular weight of 82 kDa, 80-83 kDa, 78 kDa, 76 kDa, 74 kDa, and 70 kDa as determined by SDS-PAGE. In one embodiment, the low iron condition is growth in the presence of 2,2′-dipyridyl. An example of an 82 kDa metal-regulated protein isolatable from an E. coli is SEQ ID NO:10 a (FecA protein). An example of an 80-83 kDa metal-regulated protein isolatable from an E. coli is SEQ ID NO:8 (a FepA protein). An example of a 76 kDa metal-regulated protein isolatable from an E. coli is SEQ ID NO:12 (an IutA protein). An example of a 78 kDa metal-regulated protein isolatable from an E. coli is SEQ ID NO:6 (an IroN protein). An example of a 74 kDa metal-regulated protein isolatable from an E. coli is SEQ ID NO:4 (an IreA protein). An example of a 70 kDa metal-regulated protein isolatable from an E. coli is SEQ ID NO:2 (a ChuA protein). Reference to molecular weight here refers to molecular weight as determined by resolving a protein using an SDS polyacrylamide gel having a stacking gel of about 4% and a resolving gel of about 10% under reducing and denaturing conditions. Typically, extracts of two or more microbes can be combined to achieve a composition with the six proteins. In one embodiment, one E. coli cell produces proteins having molecular weights of 80-83 kDa (a FepA protein), 78 kDa (an IroN protein), 74 kDa (an IreA protein), and 70 kDa (a ChuA protein), and a different E. coli cell produces proteins having molecular weights of 82 kDa (a FecA protein), 80-83 kDa (a FepA protein), and 76 kDa (an IutA protein).

A composition that includes proteins isolatable from one or more microbes when the microbe(s) naturally expresses the proteins during growth in low iron conditions can also include proteins that are not metal-regulated. Such proteins are expressed in the presence of a metal ion such as, for example, in the presence of ferric chloride, and are expressed when grown in low iron conditions. Examples of this type of protein isolatable from E. coli include the low molecular weight OmpC and OmpA proteins shown in FIG. 1.

Optionally, a protein described herein can be covalently bound to a carrier protein to improve the immunological properties of the protein. Useful carrier proteins are known in the art. The chemical coupling of a protein described herein can be carried out using known and routine methods. For instance, various homobifunctional and/or heterobifunctional cross-linker reagents such as bis(sulfosuccinimidyl) suberate, bis(diazobenzidine), dimethyl adipimidate, dimethyl pimelimidate, dimethyl superimidate, disuccinimidyl suberate, glutaraldehyde, m-maleimidobenzoyl-N-hydroxysuccinimide, sulfo-m-maleimidobenzoyl-N-hydroxysuccinimide, sulfosuccinimidyl 4-(N-maleimidomethyl) cycloheane-1-carboxylate, sulfosuccinimidyl 4-(p-maleimido-phenyl) butyrate and (1-ethyl-3-(dimethyl-aminopropyl) carbodiimide can be used (Harlow and Lane, Antibodies, A Laboratory Manual, generally and Chapter 5, Cold Spring Harbor Laboratory, Cold Spring Harbor, New York, NY (1988)).

A composition described herein can include low concentrations of lipopolysaccharide (LPS). LPS is a component of the outer membrane of most gram negative microbes (see, for instance, Nikaido and Vaara, Outer Membrane, In: Escherichia coli and Salmonella typhimurium, Cellular and Molecular Biology, Neidhardt et al., (eds.) American Society for Microbiology, Washington, D.C., pp. 7-22 (1987), and typically includes polysaccharides (O—specific chain, the outer and inner core) and the lipid A region. The lipid A component of LPS is the most biologically active component of the LPS structure and together induces a wide spectrum of pathophysiological effects in mammals. The most dramatic effects are fever, disseminated intravascular coagulation, complement activation, hypotensive shock, and death. The non-specific immunostimulatory activity of LPS can enhance the formation of a granuloma at the site of administration of compositions that include LPS. Such reactions can result in undue stress on the animal whereby the animal may reduce feed or water intake for a period of time and exacerbate infectious conditions in the animal. In addition, the formation of a granuloma at the site of injection can increase the likelihood of possible down grading of the carcass due to scaring or blemishes of the tissue at the injection site.

The concentration of LPS can be determined using routine methods known in the art. Such methods typically include measurement of dye binding by LPS (see, for instance, Keler and Nowotny, Analyt. Biochem., 156, 189 (1986)) or the use of a Limulus amebocyte lysate (LAL) test (see, for instance, Endotoxins and Their Detection With the Limulus Amebocyte Lystate Test, Alan R. Liss, Inc., 150 Fifth Avenue, New York, NY (1982)). There are four basic commercially available methods that are typically used with an LAL test: the gel-clot test; the turbidimetric (spectrophotometric) test; the colorimetric test; and the chromogenic test. An example of a gel-clot assay is available under the tradename E-TOXATE (Sigma Chemical Co., St. Louis, MO; see Sigma Technical Bulletin No. 210), and PYROTELL (Associates of Cape Cod, Inc., East Falmouth, MA). Typically, assay conditions include contacting the composition with a preparation containing a lysate of the circulating amebocytes of the horseshoe crab, Limulus polyphemus. When exposed to LPS, the lysate increases in opacity as well as viscosity and may gel. About 0.1 mL of the composition is added to lysate. Typically, the pH of the composition is between 6 and 8, preferably, between 6.8 and 7.5. The mixture of composition and lysate is incubated for 1 hour undisturbed at 37° C. After incubation, the mixture is observed to determine if there was gelation of the mixture. Gelation indicates the presence of endotoxin. To determine the amount of endotoxin present in the composition, dilutions of a standardized solution of endotoxin are made and tested at the same time that the composition is tested. Standardized solutions of endotoxin are commercially available from, for instance, Sigma Chemical (Catalog No. 210-SE), U.S. Pharmacopeia (Rockville, MD, Catalog No. 235503), and Associates of Cape Cod, Inc., (Catalog No. E0005). In general, when a composition of the present disclosure is prepared by isolating proteins from a microbe, such as P. multocida, by a method as described herein (e.g., a method that includes disrupting and solubilizing the cells, and collecting the insoluble proteins), the amount of LPS in a composition of the present disclosure is less than the amount of LPS present in a mixture of same amount of the microbe that has been disrupted under the same conditions but not solubilized. Typically, the level of LPS in a composition of the present disclosure is decreased by, in increasing order of preference, at least 50%, at least 60%, at least 70%, at least 80%, or at least 90% relative to the level of LPS in a composition prepared by disrupting, but not solubilizing, the same microbe.

A composition described herein optionally further includes a pharmaceutically acceptable carrier. “Pharmaceutically acceptable” refers to a diluent, carrier, excipient, salt, etc., that is compatible with the other ingredients of the composition, and not deleterious to the recipient thereof. Typically, the composition includes a pharmaceutically acceptable carrier when the composition is used as described herein. Exemplary pharmaceutically acceptable carriers include buffer solutions and generally exclude blood products such as, for example, whole blood and/or plasma. As used herein, the term “vaccine composition” and “vaccine” refers to a pharmaceutical composition containing one or more protein described herein, where the composition can be used to prevent or treat a disease or condition in a subject.

The compositions as described herein may be formulated in pharmaceutical preparations in a variety of forms adapted to the chosen route of administration, including routes suitable for stimulating an immune response to an antigen. Thus, a composition as described herein can be administered via known routes including, for example, oral; parenteral including intradermal, transcutaneous and subcutaneous, intramuscular, intravenous, intraperitoneal, etc. and topically, such as, intranasal, intrapulmonary, intramammary, intravaginal, intrauterine, intradermal, transcutaneous and rectally, etc. It is foreseen that a composition can be administered to a mucosal surface, such as by administration to the nasal or respiratory mucosa (e.g., via a spray or aerosol), in order to stimulate mucosal immunity, such as production of secretory IgA antibodies, throughout the animal's body.

A composition as described herein can also be administered via a sustained or delayed release implant. Implants suitable for use according to the disclosure are known and include, for example, those disclosed in International Publication No. WO 2001/037810 and/or International Publication No. WO 1996/001620. Implants can be produced at sizes small enough to be administered by aerosol or spray. Implants also can include nanospheres and microspheres.

A composition of the present disclosure is administered in an amount sufficient to provide an immunological response to proteins or whole cells described herein. The amount of protein present in a composition can vary. For instance, the dosage of protein can be from 0.01 micrograms (μg) to 500 μg, such as from 0.5 μg to 300 μg or from 50 μg to 200 μg. In one embodiment the dosage is 100 μg. When protein-enriched extracts derived from more than one isolate are combined, equivalent amounts of protein from each extract can be combined. When the composition is a whole cell preparation, the cells can be present at a concentration of 10⁶ bacteria/mL, 10⁷ bacteria/mL, 108 bacteria/mL, or 109 bacteria/mL. When a mixture of whole cells is administered (e.g., one population of cells expressing a subset of proteins and a second population expressing another subset of proteins) the ratio of populations can be 1:1. For an injectable composition (e.g. subcutaneous, intramuscular, etc.) the protein is preferably present in the composition in an amount such that the total volume of the composition administered is 0.05 mL to 1.0 mL, including 0.1 to 0.5 mL. Typically, one day-of-age chickens and turkeys receive a dose of 0.1 mL, but lower and higher volumes are possible. Adult chickens receive a dose of 0.25 mL to 0.5 mL, and adult turkeys receive a dose of 0.5 mL, but lower and higher volumes are possible mL. When the composition is a whole cell preparation, the cells are preferably present in the composition in an amount that the total volume of the composition administered is 0.05 mL to 1.0 mL, including 0.1 to 0.5 mL. The amount administered will vary depending on various factors including, but not limited to, the specific proteins or cells chosen, the weight, physical condition and age of the animal, and the route of administration. Thus, the absolute weight of the protein or number of cells included in a given unit dosage form can vary, and depends upon factors such as the species, age, weight and physical condition of the animal, as well as the method of administration. Such factors can be determined by one skilled in the art. Other examples of dosages suitable for the disclosure are disclosed in Emery et al. (U.S. Pat. No. 6,027,736).

The formulations may be conveniently presented in unit dosage form and may be prepared by methods well known in the art of pharmacy. All methods of preparing a composition including a pharmaceutically acceptable carrier include the step of bringing the active compound (e.g., a protein or whole cell described herein) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

A composition including a pharmaceutically acceptable carrier can also include an adjuvant. An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest. Adjuvants may include, for example, IL-1, IL-2, emulsifiers, muramyl dipeptides, dimethyldiocradecylammonium bromide (DDA), avridine, aluminum hydroxide, oils, saponins, alpha-tocopherol, polysaccharides, emulsified paraffins (available from under the tradename EMULSIGEN© from MVP Laboratories, Omaha, NE), RIBI, water-in-oil and/or oil-in-water using light mineral oil using tweens, spans or Arlacel A as emulsifiers, montanide adjuvants commercially available such as ISA 70, ISA 71 VG, ISA 78 VG, ISA 71 R-VG and/or Gel O2 PR (Sepic Inc, New Jersey), polymer-type adjuvants (available under the tradename CARBIGEN™ and POLYGEN™ from MVP Laboratories (Omaha, NE), and other substances known in the art.

In another embodiment, a composition including a pharmaceutically acceptable carrier can include a biological response modifier, such as, for example, IL-2, IL-4 and/or IL-6, TNF, IFN-alpha, IFN-gamma, and other cytokines that effect immune cells. A composition can also include a chelating agent such as succimer or deferoxamine, a preservative such as polymyxin B, formalin, or gentamicin, an anti-oxidant such as ascorbic acid or calcium stearate, etc. Such components are known in the art.

### Methods of Making

This disclosure also provides methods for obtaining the proteins and whole cells described herein. Proteins and whole cell preparations described herein may be obtained by incubating an E. coli under conditions that promote expression of one or more of the proteins described herein. In one embodiment, an E. coli that expresses FepA, IroN, IreA, and ChuA is used and a second E. coli that expresses FecA, FepA, and IutA is used and the proteins combined to result in a composition described herein. In addition, such microbes are readily obtainable by techniques routine and known in the art. The microbes may be derived from an infected animal as a field isolate, and used to obtain the proteins and/or the whole cell preparations as described herein, or stored for future use, for example, in a frozen repository at from −20° C. to −95° C., or from −40° C. to −50° C., in bacteriological media containing 20% glycerol, and other like media. The proteins and whole cells as described herein may be isolatable from one or more microbes engineered to recombinantly express one or more of the proteins.

The present disclosure also includes compositions prepared by the processes disclosed herein. Typically, such conditions are low metal conditions. As used herein, the phrase “low metal conditions” refers to an environment, typically bacteriological media that contains amounts of a free metal that cause a microbe to express a metal regulated protein at a detectable level. As used herein, the phrase “high metal conditions” refers to an environment that contains an amount of a free metal that causes a microbe to express a metal-regulated protein at a decreased level compared to expression of the metal-regulated protein under low metal conditions. In some cases, “high metal conditions” can refer to an environment that causes a cell to fail to express one or more of the metal-regulated proteins described herein at a detectable level.

In some cases, “high metal conditions” can include a metal-rich natural environment and/or culture in a metal-rich medium without a metal chelator. In contrast, in some cases, “low metal conditions” can include culture in a medium that includes a metal chelator, as described in more detail below. The metal is typically iron.

Low metal conditions are generally the result of the addition of a metal chelating compound to a bacteriological medium, the use of a bacteriological medium that contains low amounts of a metal, or a combination thereof. High metal conditions are generally present when a chelator is not present in the medium, when a metal is added to the medium, or a combination thereof. Examples of metal chelators include natural and synthetic compounds. Examples of natural compounds include plant phenolic compounds, such as flavonoids. Examples of flavonoids include the iron chelator myricetin. Examples of synthetic iron chelators include 2,2′-dipyridyl (also referred to in the art as α,α′-bipyridyl), 8-hydroxyquinoline, ethylenediamine-di-O-hydroxyphenylacetic acid (EDDHA), desferrioxamine methanesulfonate (desferol), transferrin, lactoferrin, ovotransferrin, biological siderophores, such as the catecholates and hydroxamates, and citrate.

In one embodiment, 2,2′-dipyridyl is used for the chelation of iron. Typically, 2,2′-dipyridyl is added to the media at a concentration of at least 0.0025 micrograms/milliliter (μg/mL), at least 0.025 μg/mL, or at least 0.25 μg/mL. High levels of 2,2′-dipyridyl can be 10 μg/mL, 20 μg/mL, or 30 μg/mL.

It is expected that an E. coli with a mutation in a fur gene will result in the constitutive expression of metal regulated proteins. The production of a fur mutation in an E. coli can be produced using routine methods including, for instance, transposon, chemical, or site-directed mutagenesis useful for generating gene knock-out mutations in gram negative bacteria.

In one embodiment, the E. coli used to make a composition described herein, e.g., a composition including isolated proteins or a composition including whole cells, may be produced using one or more E. coli that has been engineered to recombinantly express a protein that is identical to or has structural similarity with SEQ ID NO:2 or 13, is identical to or has structural similarity with SEQ ID NO:4 or 14, is identical to or has structural similarity with SEQ ID NO:6 or 15, is identical to or has structural similarity with SEQ ID NO:8 or 16, is identical to or has structural similarity with SEQ ID NO:10 or 17, is identical to or has structural similarity with SEQ ID NO:12 or 18, is identical to or has structural similarity with SEQ ID NO:20 or 23, is identical to or has structural similarity with SEQ ID NO:22 or 24, is identical to or has structural similarity with SEQ ID NO:44 or 45, or a combination thereof. In one embodiment, an E. coli that naturally produces one or more of the proteins disclosed herein is engineered to produce one or more additional proteins disclosed herein, and the one of more natural metal-regulated proteins and one or more of the recombinant proteins are expressed during the incubation of the engineered E. coli in the low iron conditions. The result is an E. coli that expresses metal-regulated proteins and the one of more recombinant proteins.

The medium used to incubate the microbe is not critical, and conditions useful for the culture of E. coli are known to the skilled person. The volume of medium used to incubate the microbe can vary. When an E. coli is being evaluated for the ability to produce the proteins described herein, the microbe can be grown in a suitable volume, for instance, 10 mL to 1 L of medium. When a microbe is being grown to obtain proteins for use in, for instance, administration to animals, the microbe may be grown in a fermenter to allow the isolation of larger amounts of proteins. Methods for growing microbes in a fermenter are routine and known in the art. The conditions used for growing a microbe preferably include a metal chelator such as desferal, deferoxamine, deferasirox, deferiprone, ethylene di-ortho-hydroxyphenyl—acetic acid (EDDA) and/or ethylenediaminetetraacetic acid (EDTA) more preferably an iron chelator, for instance 2,2′-dipyridyl or 2,2′-bipyridyl, a pH of between 6.5 and 7.5, preferably between 6.9 and 7.1, and a temperature of 37° C.

In some aspects of the disclosure, an E. coli may be harvested after growth. Harvesting includes concentrating the microbe into a smaller volume and suspending in a media different than the growth media. Methods for concentrating a microbe are routine and known in the art, and include, for example, filtration and/or centrifugation. Typically, the concentrated microbe is suspended in decreasing amounts of buffer. Preferably, the final buffer includes a metal chelator, preferably, ethylenediaminetetraacetic acid (EDTA). An example of a buffer that can be used contains Tris-base (7.3 grams/liter) and EDTA (0.9 grams/liter), at a pH of 8.5. Optionally, the final buffer also minimizes proteolytic degradation. This can be accomplished by having the final buffer at a pH of greater than 8.0, preferably, at least 8.5, and/or including one or more proteinase inhibitors (e.g., phenylmethanesulfonyl fluoride). Optionally and preferably, the concentrated microbe is frozen at −20° C. or below until disrupted. In one embodiment, bacterial cells may be concentrated into a pellet by, for instance, centrifugation, and the concentrated cells suspended in osmotic shock buffer (OMS; 7.26 grams/liter Tris-base and 0.93 grams/liter EDTA adjusted to a pH of 8.5). The ratio of cells to OMS may be 50 grams cell pellet, 60 grams cell pellet, or 70 grams cell pellet to 1 liter of OMS. The suspension of cells in OMS can be incubated at 2-8° C. for at least 24 hours, at least 48 hours, or at least 60 hours to remove excess endotoxin from the cells. In one embodiment, the incubation is for no greater than 72 hours. After the incubation the suspension is centrifuged again and the supernatant discarded to remove free endotoxin and any extracellular material, e.g., secreted proteins.

When the E. coli is to be used as a whole cell preparation, the harvested cells may be processed using routine and known methods to inactivate the cells. Alternatively, when an E. coli is to be used to prepare proteins of the present disclosure, the E. coli may be disrupted using chemical, physical, or mechanical methods routine and known in the art, including, for example, french press, sonication, or homogenization. Preferably, homogenization is used. As used herein, “disruption” refers to the breaking up of the cell. Disruption of a microbe can be measured by methods that are routine and known in the art, including, for instance, changes in optical density. Typically, a microbe is subjected to disruption until the percent transmittance is increased by 20% when a 1:100 dilution is measured. The temperature during disruption is typically kept at 4° C., to further minimize proteolytic degradation.

The disrupted microbe is solubilized in a detergent, for instance, an anionic, zwitterionic, nonionic, or cationic detergent. Preferably, the detergent is sarcosine, more preferably, sodium lauroyl sarcosinate. As used herein, the term “solubilize” refers to dissolving cellular materials (e.g., proteins, nucleic acids, carbohydrates) into the aqueous phase of the buffer in which the microbe was disrupted, and the formation of aggregates of insoluble cellular materials. The conditions for solubilization preferably result in the aggregation of proteins of the present disclosure into insoluble aggregates that are large enough to allow easy isolation by, for instance, centrifugation or filtration.

Preferably, the sarcosine is added such that the final ratio of sarcosine to gram weight of disrupted microbe is between 1.0 gram sarcosine per 4.5 grams pellet mass and 6.0 grams sarcosine per 4.5 grams pellet mass, preferably, 4.5 gram sarcosine per 4.5 grams pellet mass. The solubilization of the microbe may be measured by methods that are routine and known in the art, including, for instance, changes in optical density. Typically, the solubilization is allowed to occur for at least 24 hours, more preferably, at least 48 hours, most preferably, at least 60 hours. The temperature during disruption is typically kept low, preferably at 4° C.

The insoluble aggregates that include the proteins described herein may be isolated by methods that are routine and known in the art, such as centrifugation, filtration, or a combination thereof. In one embodiment, the insoluble aggregates are isolated by filtration, such as tangential or crossflow filtration. Examples of a molecular weight cutoff to use with tangential filtration are 40 kDa, 50 kDa, or 60 kDa. In one embodiment, a tangential filtration system has a molecular weight cutoff of 50 kDa. Tangential filtration may aid in removal of residual sarcosine from the protein suspension. Tangential filtration results in concentration of the protein suspension. Thus, the insoluble aggregates can be isolated at a significantly lower cost.

In one embodiment, the sarcosine is removed from the isolated proteins. Methods for removing sarcosine from the isolated proteins are known in the art, and include, for instance, diafiltration, precipitation, hydrophobic chromatography, ion-exchange chromatography, and/or affinity chromatography, and ultrafiltration and washing the proteins in alcohol, such as isopropyl alcohol, by diafiltration. After isolation, the proteins suspended in buffer and stored at low temperature, for instance, −20° C. or below.

Proteins of the present disclosure may also be isolated from microbes using methods that are known to the art. The isolation of the proteins may be accomplished as described in, for instance, Emery et al. (U.S. Pat. No. 7,147,857).

In those aspects of the present disclosure where a whole cell preparation is to be made, after growth of an E. coli the microbe can be killed using heat or by the addition of an agent such as glutaraldehyde or formalin, at a concentration sufficient to inactivate the cells in the culture. For instance, formalin can be added at a concentration of 0.3% (vol:vol). After a period of time sufficient to inactivate the cells, the cells can be harvested by, for instance, diafiltration and/or centrifugation, and washed.

In other aspects, an isolated protein of the disclosure may be prepared recombinantly. When prepared recombinantly, a polynucleotide encoding the protein may be identified and cloned into an appropriate expression host. The recombinant expression host may be grown in an appropriate medium, disrupted, and the proteins isolated as described above.

### Methods of Use

Also provided are methods of using the proteins described herein. The methods include administering to an animal an effective amount of a composition that includes at least one, at least two, at least three, at least four, at least five, at least six, at least seven, or at least eight proteins described herein. The proteins can be isolated or present in a preparation of one or more whole cells. The composition may further include a pharmaceutically acceptable carrier. As used herein, an “effective amount” of a composition of the present disclosure is the amount able to elicit the desired response in the recipient. The composition can be administered at a time that maternal antibody may be present, for instance, as early as one day of age, or at a later time during the life of the animal. The animal can be, for instance, an avian such as, for instance, turkeys, chickens, and ducks. In some embodiments, the chicken can be a layer, a broiler breeder, or a broiler. In some embodiments, the turkey can be a breeder, a commercial bird, or a grandparent stock. In some aspects, the methods may further include additional administrations (e.g., one or more booster administrations) of the composition to the animal to enhance or stimulate a secondary immune response. A booster can be administered at a time after the first administration, for instance, 1 to 8 weeks, preferably 2 to 4 weeks, after the first administration of the composition. Subsequent boosters can be administered one, two, three, four, or more times annually. Without intending to be limited by theory, it is expected that annual boosters will not be necessary, as an animal will be challenged in the field by exposure to E. coli expressing proteins having epitopes that are identical to or structurally related to epitopes present on the proteins present in the composition administered to the animal.

In one aspect, the disclosure is directed to methods for making antibody to a protein described herein, for instance, by inducing the production of antibody in an animal, or by recombinant techniques. The antibody produced includes antibody that specifically binds at least one protein present in the composition. In this aspect of the disclosure, an “effective amount” is an amount effective to result in the production of antibody in the animal. Methods for determining whether an animal has produced antibodies that specifically bind proteins present in a composition of the present disclosure can be determined as described herein.

As used herein, an antibody that can “specifically bind” a protein is an antibody that interacts only with the epitope of the antigen that induced the synthesis of the antibody, or interacts with a structurally related epitope. An antibody that “specifically binds” to an epitope will, under the appropriate conditions, interact with the epitope even in the presence of a diversity of potential binding targets.

In one aspect the disclosure is also directed to treating an infection in an animal caused by an E. coli. The method includes administering an effective amount of the composition to an animal having an infection caused by an E. coli and determining whether the E. coli causing the infection has decreased. Methods for determining whether an infection is caused by an E. coli are routine and known in the art.

In another aspect, the present disclosure is directed to methods for treating one or more signs of certain conditions in animals that may be caused by infection by an E. coli. Examples of conditions caused by E. coli infections include peritonitis, including peritonitis of chickens and/or turkeys; localized colibacillosis, including infection of air sacs, liver, heart, and/or spleen of chickens and/or turkeys; and septicemia. Examples of signs of avian peritonitis are known to the skilled person and include, for instance, reduced egg production and/or the presence of E. coli in oviduct and/or ovary. Examples of signs of localized colibacillosis are known to the skilled person and include the presence of E. coli in air sacs, liver, heart, and/or spleen of chickens and/or turkeys. Examples of signs of septicemia are known to the skilled person and include the presence of E. coli in the bloodstream.

Treatment of these conditions can be prophylactic or, alternatively, can be initiated after the development of a condition described herein. Treatment that is prophylactic, for instance, initiated before a subject manifests signs of a condition caused by E. coli is referred to herein as treatment of a subject that is “at risk” of developing the condition. Typically, an animal “at risk” of developing a condition is an animal likely to be exposed to an E. coli causing the condition. For instance, the animal is present in an area where the condition has been diagnosed in at least one other animal, and/or is being transported to an area where pathogenic E. coli is endemic, and/or where conditions caused by E. coli are prevalent. Accordingly, administration of a composition can be performed before, during, or after the occurrence of the conditions described herein. Treatment initiated before the development of a condition may result in preventing the signs of one of the conditions from occurring in an animal exposed to E. coli. Treatment initiated after the development of a condition may result in decreasing the severity of the signs of one of the conditions, including completely removing the signs. In this aspect of the disclosure, an “effective amount” is an amount effective to prevent the manifestation of signs of a condition, or decrease the severity of the signs of a condition, and/or completely remove the signs.

The potency of a composition described herein can be tested according to standard methods. For instance, the use of a chicken model of avian pathogen E. coli peritonitis is established (Chaudhari and Kariyawasam, Avian Dis. 58:25-33, 2014; Huja et al., mBio. 6:1-13, 2015; Cox et al., Avian Diseases, 65(1):198-204, 2020, and Example 12). These models can also be used to evaluate colonization of air sac, liver, heart, and spleen. The use of a murine model for septicemia is established (Koutsianos et al., 2020 Vet. Sci, 7(3):80 Pages 2-12).

A composition of the disclosure can be used to provide for passive immunization against infection by E. coli. For instance, the composition can be administered to an animal to induce the production of immune products, such as antibodies, which can be collected from the producing animal and administered to another animal to provide passive immunity. Immune components, such as antibodies, can be collected to prepare antibody compositions from serum, plasma, blood, colostrum, etc. for passive immunization therapies. Antibody compositions including monoclonal antibodies, anti-idiotypes, and/or recombinant antibodies can also be prepared using known methods. Passive antibody compositions and fragments thereof, e.g., scFv, Fab, F(ab′)₂ or Fv or other modified forms thereof, may be administered to a recipient in the form of serum, plasma, blood, colostrum, and the like. However, the antibodies may also be isolated from serum, plasma, blood, colostrum, and the like, using known methods and spray dried or lyophilized for later use in a concentrated or reconstituted form. Passive immunizing preparations may be particularly advantageous for treatment of acute systemic illness, or passive immunization of young animals that failed to receive adequate levels of passive immunity through maternal colostrum.

Another aspect of the present disclosure provides methods for detecting antibody that specifically binds proteins of the present disclosure. These methods are useful in, for instance, detecting whether an animal has antibody that specifically binds proteins of the present disclosure, and diagnosing whether an animal may have an infection caused by E. coli. Preferably, such diagnostic systems are in kit form. The methods include contacting an antibody with a preparation that includes at least one protein of the present disclosure to result in a mixture. Preferably, the antibody is present in a biological sample, such as blood.

The method further includes incubating the mixture under conditions to allow the antibody to specifically bind a protein to form a protein:antibody complex. As used herein, the term “protein:antibody complex” refers to the complex that results when an antibody specifically binds to a protein. The preparation that includes the proteins present in a composition of the present disclosure may also include reagents, for instance a buffer, that provide conditions appropriate for the formation of the protein:antibody complex. The protein:antibody complex is then detected. The detection of antibodies is known in the art and can include, for instance, immunofluorescence and peroxidase.

The methods for detecting the presence of antibodies that specifically bind to proteins of the present disclosure can be used in various formats that have been used to detect antibody, including radioimmunoassay and enzyme-linked immunosorbent assay.

The present disclosure also provides a kit for detecting antibody that specifically binds proteins of the present disclosure. The kit includes at least one protein of the present disclosure in a suitable packaging material in an amount sufficient for at least one assay. Optionally, other reagents such as buffers and solutions needed to practice the disclosure are also included. Instructions for use of the packaged proteins are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the proteins can be used for detecting antibodies induced by infection with E. coli. In addition, the packaging material contains instructions indicating how the materials within the kit are employed to detect such antibodies. As used herein, the term “package” refers to a solid matrix or material such as glass, plastic, paper, foil, and the like, capable of holding within fixed limits the proteins. Thus, for example, a package can be a microtiter plate well to which microgram quantities of proteins have been affixed. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is a composition that comprises an isolated ChuA protein, such as a protein that has at least 80% identity with SEQ ID NO:2, an isolated IreA protein, such as a protein that has at least 80% identity with SEQ ID NO:4, an isolated IroN protein, such as a protein that has at least 80% identity with SEQ ID NO:6, an isolated FepA protein, such as a protein that has at least 80% identity with SEQ ID NO:8, an isolated FecA protein, such as a protein that has at least 80% identity with SEQ ID NO:10, an isolated IutA protein, such as a protein that has at least 80% identity with SEQ ID NO:12, a pharmaceutically acceptable carrier, and an adjuvant.

Aspect 2 is the composition of aspect 1, that further comprises an isolated BtuB protein, such as a protein that has at least 80% identity with SEQ ID NO:20, an isolated CirA protein, such as a protein that has at least 80% identity with SEQ ID NO:22, or an isolated BtuB protein and an isolated CirA protein.

Aspect 3 is a composition that comprises any two, three, four, five, six, seven, or eight of the proteins chosen from: an isolated ChuA protein, such as a protein that has at least 80% identity with SEQ ID NO:2, an isolated IreA protein, such as a protein that has at least 80% identity with SEQ ID NO:4, an isolated IroN protein, such as a protein that has at least 80% identity with SEQ ID NO:6, an isolated FepA protein, such as a protein that has at least 80% identity with SEQ ID NO:8, an isolated FecA protein, such as a protein that has at least 80% identity with SEQ ID NO:10, an isolated IutA protein, such as a protein that has at least 80% identity with SEQ ID NO:12, an isolated BtuB protein, such as a protein that has at least 80% identity with SEQ ID NO:20, an isolated CirA protein, such as a protein that has at least 80% identity with SEQ ID NO:22, and an isolated FyuA protein, such as a protein that has at least 80% identity with SEQ ID NO:44, the composition further comprising a pharmaceutically acceptable carrier and an adjuvant.

Aspect 4 is a composition comprising: an isolated IreA protein, such as a protein that has at least 80% identity with SEQ ID NO:4, an isolated ChuA protein, such as a protein that has at least 80% identity with SEQ ID NO:2, an isolated FepA protein, such as a protein that has at least 80% identity with SEQ ID NO:8, and an isolated IroN protein, such as a protein that has at least 80% identity with SEQ ID NO:6; an isolated ChuA protein, such as a protein that has at least 80% identity with SEQ ID NO:2, an isolated IroN protein, such as a protein that has at least 80% identity with SEQ ID NO:6, and an isolated FyuA protein, such as a protein that has at least 80% identity with SEQ ID NO:44; an isolated IreA protein, such as a protein that has at least 80% identity with SEQ ID NO:4 and an isolated ChuA protein, such as a protein that has at least 80% identity with SEQ ID NO:2; an isolated FepA protein, such as a protein that has at least 80% identity with SEQ ID NO:8 and an isolated IroN protein, such as a protein that has at least 80% identity with SEQ ID NO:6; or an isolated ChuA protein, such as a protein that has at least 80% identity with SEQ ID NO:2 and an isolated IroN protein, such as a protein that has at least 80% identity with SEQ ID NO:6; the composition further comprising a pharmaceutically acceptable carrier and an adjuvant.

Aspect 5 is a method comprising administering to a subject an amount of the composition of any one of aspects 1 to 4, 21, or 22, or the whole cell of aspects 19 or 20 effective to induce the subject to produce antibody that specifically binds to at least one protein of the composition.

Aspect 6 is a method for treating peritonitis in a subject, the method comprising administering an effective amount of the composition of any one of aspects 1 to 4, 21, or 22, or the whole cell of aspects 19 or 20 to a subject having or at risk of having peritonitis caused by E. coli, wherein the subject is a domesticated fowl.

Aspect 7 is a method for treating a sign of peritonitis in a subject, the method comprising administering an effective amount of the composition of any one of aspects 1 to 4, 21, or 22, or the whole cell of aspects 19 or 20 to a subject having or at risk of having peritonitis caused by E. coli, wherein the subject is a domesticated fowl.

Aspect 8 is a method for treating localized colibacillosis in a subject, the method comprising administering an effective amount of the composition of any one of aspects 1 to 4, 21, or 22, or the whole cell of aspects 19 or 20 to a subject having or at risk of having localized colibacillosis caused by E. coli, wherein the subject is a domesticated fowl.

Aspect 9 is a method for treating a sign of localized colibacillosis in a subject, the method comprising administering an effective amount of the composition of any one of aspects 1 to 4, 21, or 22, or the whole cell of aspects 19 or 20 to a subject having or at risk of having localized colibacillosis caused by E. coli, wherein the subject is a domesticated fowl.

Aspect 10 is a method for treating septicemia in a subject, the method comprising administering an effective amount of the composition of any one of aspects 1 to 4, 21, or 22, or the whole cell of aspects 19 or 20 to a subject having or at risk of having septicemia caused by E. coli, wherein the subject is a domesticated fowl.

Aspect 11 is a method for treating a sign of septicemia in a subject, the method comprising administering an effective amount of the composition of any one of aspects 1 to 4, 21, or 22, or the whole cell of aspects 19 or 20 to a subject having or at risk of having septicemia caused by E. coli, wherein the subject is a domesticated fowl.

Aspect 12 is a method for treating peritonitis in a subject, the method comprising administering an effective amount of a composition to a subject having or at risk of having peritonitis caused by E. coli, wherein the composition comprises antibody that specifically binds to a protein of the composition of any one of aspects 1 to 4, wherein the subject is a domesticated fowl.

Aspect 13 is a method for treating localized colibacillosis in a subject comprising administering an effective amount of a composition to a subject having or at risk of having localized colibacillosis caused by E. coli, wherein the composition comprises antibody that specifically binds to a protein of the composition of any one of aspects 1 to 4, wherein the subject is a domesticated fowl.

Aspect 14 is a method for treating a sign of localized colibacillosis in a subject, the method comprising administering an effective amount of a composition to a subject having or at risk of having localized colibacillosis caused by E. coli, wherein the composition comprises antibody that specifically binds to a protein of the composition of any one of aspects 1 to 4, wherein the subject is a domesticated fowl.

Aspect 15 is a method for treating septicemia in a subject comprising administering an effective amount of a composition to a subject having or at risk of having septicemia caused by E. coli, wherein the composition comprises antibody that specifically binds to a protein of the composition of any one of aspects 1 to 4, wherein the subject is a domesticated fowl.

Aspect 16 is a method for treating a sign of septicemia in a subject, the method comprising administering an effective amount of a composition to a subject having or at risk of having septicemia caused by E. coli, wherein the composition comprises antibody that specifically binds to a protein of the composition of any one of aspects 1 to 4, wherein the subject is a domesticated fowl.

Aspect 17 is the method of any one of aspects 5 to 16, wherein the domesticated fowl is a chicken, a turkey, or a duck.

Aspect 18 is the method of any one of aspects 5 to 17, wherein at least 0.01 micrograms (μg) and no greater than 500 μg of protein is administered.

Aspect 19 is an isolated whole cell engineered to express six proteins, wherein the six proteins are an isolated ChuA protein, such as a protein that has at least 80% identity with SEQ ID NO:2, an isolated IreA protein, such as a protein that has at least 80% identity with SEQ ID NO:4, an isolated IroN protein, such as a protein that has at least 80% identity with SEQ ID NO:6, an isolated FepA protein, such as a protein that has at least 80% identity with SEQ ID NO:8, an isolated FecA protein, such as a protein that has at least 80% identity with SEQ ID NO:10, an isolated IutA protein, such as a protein that has at least 80% identity with SEQ ID NO:12.

Aspect 20 is the whole cell of aspect 19, wherein the cell is E. coli.

Aspect 21 is a composition comprising two or more populations of microbes, wherein each of the populations express a subset of the six proteins of aspect 20, and the two or more populations considered as a whole express the six proteins.

Aspect 223 is the composition of aspect 21, wherein the microbes are E. coli.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Evaluation of E. coli Isolates for Production of Metal Regulated Proteins

To further examine and to get a better understanding of SRP protein expression, the banding profiles of 440 avian pathogenic E. coli (APEC) field isolates were determined. The E. coli were isolated from infected organs (liver, spleen and/or oviducts) derived from chickens and turkeys obtained from multiple commercial facilities throughout the United States showing clinical signs of peritonitis or related clinical signs of colibacillosis.

To obtain a better perspective of the up-regulation of metal-regulated proteins of each E. coli isolate, they were grown in iron replete and iron deplete media conditions. Briefly, the organisms were grown by sub-culturing into two separate 500 mL bottles. One bottle contained 200 mL of sterile TSB containing 300 μM 2,2-diprydyl (Sigma-Aldrich St. Louis, MO) while the second bottle contained 200 mL of Tryptic Soy broth containing 200 μM ferric chloride (Sigma-Aldrich St. Louis, MO). Cultures were incubated for 12 hours with continuous stirring at 200 rpm at 37° C. Following the 12-hour incubation period, the cultures were sub-cultured (1:100) into 500 mL of either the iron-replete and/or the iron-deplete media and incubated at 37° C. for 8 hours. After 8 hours each culture was centrifuged at 10,000×g for 20 minutes and resuspended in 40 mL of osmotic shock buffer (7.3 g/l Tris Base; 1.86 g/l EDTA), pH 8.9. The suspensions were centrifuged at 32,000×g for 12 minutes to clarify or remove large cellular debris. The supernatants were collected and solubilized by the addition of 4% sodium lauroyl sarcosinate at 4° C. for 24 hours. The detergent-insoluble outer membrane protein-enriched fractions were collected by centrifugation at 32,000×g for 2.5 hours at 4° C. The protein pellets were resuspended in 200 μl Tris-buffer (pH 7.2).

The protein-enriched extracts derived from each isolate were size-fractionated on SDS-PAGE gels using a 4% stacking gel and 10% resolving gel. Samples for electrophoresis were prepared by combining 10 μl of sample with 30 μl of SDS reducing sample buffer (62.5 mM Tris-HCL pH 6.8, 20% glycerol, 2% SDS, 5% β-mercaptoethanol) and boiled for 4 minutes. Samples were electrophoresed at 18 mA constant current for 5 hours at 4° C. using a Protein II xi cell power supply (BioRad Laboratories, Richmond, CA, model 1000/500).

Visual comparison of the SRP banding profiles of the 440 isolates examined by SDS-PAGE showed the number of bands (1, 2, 3, 4, 5, 6, or 7) expressed by each isolate in this diverse population. Table 2 shows the number of SRP bands expressed by the individual E. coli isolates examined. For example, 9 isolates expressed one SRP band whereas 87 isolates expressed two, 130 expressed three, 106 expressed four, 58 expressed five, 48 expressed six, and only two isolates expressed seven SRP bands (Table 2). Isolates expressing 3 and 4 SRP bands represented 54% of the total population of isolates examined.

TABLE 2
Number of SRP Bands Expressed by Individual E. coli
Isolates as Examined by Gel Electrophoresis
SRP Banding Profiles (number of SRP expressed)

| | 1 | 2 | 3 | 4 | 5 | 6 | 7 |
|---|---|---|---|---|---|---|---|
| No. of Isolates | 9 | 87 | 130 | 106 | 58 | 48 | 2 |
| % of Total | 2.1 | 19.8 | 29.5 | 24.1 | 13.2 | 10.9 | 0.45 |

Table 2 shows the number of SRP bands expressed by individual E. coli isolates as examined by gel electrophoresis. Four hundred and forty (N = 440) individual E. coli isolates were examined. Row 1 shows the number of E. coli isolates expressing one to seven SRPs. Row 2 shows the percent of the population (440 isolates examined) expressing one to seven SRP bands.

Two isolates were chosen (isolate APEC-1966 and isolate APEC-1967) on the basis of their SRP banding profile which, together, represented the commonality of banding patterns that contained many of the SRPs in those isolates that induced peritonitis. Isolate APEC-1966 was serotype 0156 and isolate APEC-1967 was serotype O78. We hypothesized that use of a composition of bands present in most of the avian pathogenic E. coli examined would yield a vaccine providing broader protection, that is, protection against more serotypes than the serotypes 0156 and O78, against peritonitis in the field. Mass spectrometry analyses of trypsin fragments of gel-isolated SRP from these isolates collectively identified the following SRP proteins: ChuA, IroN, IreA, IutA, FecA and FepA. Genomic analyses by PCR indicated that genes for other SRPs (FhuE, Fiu, CirA FhuA, FyuA and BtuB) exist in these isolates, but it is unknown if they are expressed during iron-restricted antigen production.

### Example 2

Characterization of Metal Regulated Proteins of Multiple E. coli Field Isolates

The E. coli protein banding profiles of the APEC-1966 and APEC-1967 were characterized using matrix assisted laser desorption/ionization time-of-flight mass spectrometry (MALDI-TOF MS). After the proteins of the individual E. coli isolates had been resolved using a sodium dodecyl sulfate-polyacrylamide gel electrophoresis, the gel was stained with either Coomassie brilliant blue or silver to visualize the proteins.

### Materials and Methods

Excision and washing. After resolving proteins using SDS-PAGE and staining to visualize the proteins, the gel was washed for 10 minutes with water twice. Each protein band of interest was excised by cutting as close to the protein band as possible to reduce the amount of gel present in the sample. Eight gel fragments were prepared using the eight bands identified in FIG. 1 as FepA, IroN, IreA, ChuA, FecA, IutA, OmpC, and OmpA.

Each gel slice was cut into 1×1 mm cubes and placed in 1.5 mL tube. The gel pieces were washed with water for 15 minutes. All the solvent volumes used in the wash steps were approximately equal to twice the volume of the gel slice. The gel slice was next washed with water/acetonitrile (1:1) for 15 minutes. When the proteins had been stained with silver, the water/acetonitrile mixture was removed, the gel pieces dried in a SpeedVac (ThermoSavant, Holbrook, NY) and then reduced and alkylated as described below. When the gel pieces were not silver-stained, the water/acetonitrile mixture was removed, and acetonitrile was added to cover until the gel pieces turned a sticky white, at which time the acetonitrile was removed. The gel pieces were rehydrated in 100 mM NH₄HCO₃, and after 5 minutes, a volume of acetonitrile equal to twice the volume of the gel pieces was added. This was incubated for 15 minutes, the liquid removed, and the gel pieces dried in a SpeedVac.

Reduction and alkylation. The dried gel pieces were rehydrated in 10 mM DTT and 100 mM NH₄HCO₃, and incubated for 45 minutes at 56° C. After allowing the tubes to cool to room temperature, the liquid was removed and the same volume of a mixture of 55 mM iodoacetamide and 100 mM NH₄HCO₃ was immediately added. This was incubated for 30 minutes at room temperature in the dark. The liquid was removed, acetonitrile was added to cover until the gel pieces turned a sticky white, at which time the acetonitrile was removed. The gel pieces were rehydrated in 100 mM NH₄HCO₃, and after 5 minutes, a volume of acetonitrile equal to twice the volume of the gel pieces was added. This was incubated for 15 minutes, the liquid removed, and the gel pieces dried in a Speed vac. If the gel was stained with Coomassie blue, and residual Coomassie still remained, the wash with 100 mM NH₄HCO₃/acetonitrile was repeated.

In-gel digestion. Gel pieces were completely dried down in a Speed Vac. The pieces were rehydrated in digestion buffer (50 mM NH₄HCO₃, 5 mM CaCl₂), 12.5 nanograms per microliter (ng/μl) trypsin) at 4° C. Enough buffer was added to cover the gel pieces, and more was added as needed. The gel pieces were incubated on ice for 45 minutes, and the supernatant removed and replaced with 5-2 μl of same buffer without trypsin. This was incubated at 37° C. overnight in an air incubator.

Extraction of peptides. A sufficient volume of 25 mM NH₄HCO₃ was added to cover gel pieces and incubated for 15 minutes (typically in a bath sonicator). The same volume of acetonitrile was added and incubated for 15 minutes (in a bath sonicator if possible), and the supernatant was recovered. The extraction was repeated twice, using 5% formic acid instead of NH₄HCO₃. A sufficient volume of 5% formic acid was added to cover gel pieces and incubated for 15 minutes (typically in a bath sonicator). The same volume of acetonitrile was added and incubated for 15 minutes (typically in a bath sonicator), and the supernatant was recovered. The extracts were pooled, and 10 mM DTT was added to a final concentration of 1 mM DTT. The sample was dried in a SpeedVac to a final volume of approximately 5 μl.

Desalting of peptides. The samples were desalted using a ZIPTIP pipette tips (C18, Millipore, Billerica, MA) as suggested by the manufacturer. Briefly, a sample was reconstituted in reconstitution solution (5:95 acetonitrile:H₂O, 0.1% -0.5% trifluoroacetic acid), centrifuged, and the pH checked to verify that it was less than 3. A ZIPTIP was hydrated by aspirating 10 μl of solution 1 (50:50 acetonitrile:H₂O, 0.1% trifluoroacetic acid) and discarding the aspirated aliquots. This was followed by aspirating 10 μl of solution 2 (0.1% trifluoroacetic acid in deionized H₂O) and discarding the aspirated aliquots. The sample was loaded into the tip by aspirating 10 μl of the sample slowly into the tip, expelling it into the sample tube, and repeating this 5 to 6 times. Ten microliters of solution 2 was aspirated into the tip, the solution discarded by expelling, and this process was repeated 5-7 times to wash. The peptides were eluted by aspirating 2.5 μl of ice cold solution 3 (60:40, acetonitrile:H₂O, 0.1% trifluoroacetic acid), expelling, and then re-aspirating the same aliquot in and out of the tip 3 times. After the solution has been expelled from the tip, the tube was capped and stored on ice.

Mass spectrometric peptide mapping. The peptides were suspended in 10 μL to 30 μL of 5% formic acid, and analyzed by MALDI-TOF MS (Bruker Daltonics Inc., Billerica, MA). The mass spectrum of the peptide fragments was determined as suggested by the manufacturer. Briefly, a sample containing the peptides resulting from a tryptic digest were mixed with matrix cyano-4-hydroxycinnamic acid, transferred to a target, and allowed to dry. The dried sample was placed in the mass spectrometer, irradiated, and the time of flight of each ion detected and used to determine a peptide mass fingerprint for each protein present in the composition. Known proteins were used to standardize the machine.

Data analysis. The experimentally observed masses for the peptides in each mass spectrum were compared to the expected masses of proteins using the Peptide Mass Fingerprint search method of the Mascot search engine (Matrix Science Ltd., London, UK, and www.matrixscience.com, see Perkins et al., 1999, Electrophoresis 20, 3551-3567). The search parameters included: database, NCBInr; taxonomy, bacteria (eubacteria); type of search, peptide mass fingerprint; enzyme, trypsin; fixed modifications, carbamidomethyl (C) or none; variable modifications, oxidation (M), carbamidomethyl (C), the combination, or none; mass values, monoisotopic; protein mass, unrestricted; peptide mass tolerance, between ±100 ppm and ±300 ppm or 450 ppm, or ±1 Da; peptide charge state, Mr; max missed cleavages, 0 or 1; number of queries, 25.

SDS-PAGE analysis of the proteins indicated that, under the SDS-PAGE conditions used, proteins derived from 1966 and 1967 migrated at 83 kDa, 82 kDa, 78 kDa, 76 kDa, 74 kDa 70 kDa, and lower molecular weight proteins of 36 kDa to 33 kDa determined by SDS-PAGE (Table 1). MALDI analysis and predicted molecular weight based on amino acid sequence showed there was good agreement between the molecular weights of the proteins as estimated using SDS-PAGE and using MALDI (Table 1). Of the multiple E. coli isolates screened for metal regulated protein expression, six strains were selected for the following experimental studies based on their metal regulated protein expression. The avian isolates were given the following designations; APEC-1966, APEC-1967, APEC-O78, APEC-O1 and APEC-O2 derived from poultry. The APEC-01, APEC-02, and APEC-O78 isolates had 3, 5, and 4 bands when examined by SDS-PAGE as described in Example 1. In addition, we selected the uropathogenic E. coli (UPEC) isolate CFTO73 as a challenge strain to be used to evaluate the efficacy of individual recombinant proteins in a murine mouse model that represented the peritonitis composition. MALDI analysis identified the majority of the iron-regulated proteins that were found in APEC isolates to include; BtuB, CirA, FhuE, Fiu, and FyuA in addition to ChuA, FepA, FhuA, IreA, IutA, IroN,

### Example 3

Preparation of E. coli Seed Stocks

To preserve the original isolates of APEC-1966, APEC-1967, APEC-O78, APEC-01, APEC-O2 and CFTO73 a master seed stock of each isolate was prepared by inoculating the appropriate isolate into 200 mL of tryptic soy broth (TSB, Difco Laboratories, Detroit, Mich.) containing 0.34 g/L 2,2-dipyridyl (Sigma-Aldrich St. Louis, Mo.). The culture was grown while stirring at 200 rpm for 6 hours at 37° C. and collected by centrifugation at 10,000×g. The bacterial pellet was re-suspended into 100 mL TSB broth containing 20% glycerol, and sterilely dispensed into 2 mL cryogenic vials (1 mL per vial) and stored at −90° C. until use. The master seed stock was expanded into a working seed. One vial of the previously prepared master seed was inoculated into 200 mL TSB, containing 300 μM 2,2-dipyridyl (Sigma). The culture was grown while stirring at 200 rpm for 6 hours at 37° C. and collected by centrifugation at 10,000×g. The bacterial pellet was resuspended into 100 mL TSB broth containing 20% glycerol, and sterilely dispensed into 2 mL cryogenic vials (1 mL per vial) and stored at −90° C. until use.

### Example 4

Selection of E. coli Challenge Strains

Of the multiple isolates of E. coli collected and examined of Example 1, two of the isolates designated as APEC-1966 and APEC-1967 were selected based on their SRP banding profiles. The APEC isolates APEC-O78, APEC-01, and APEC-O2 were selected and evaluated for their virulence characteristics of for inducing lesions of peritonitis and ability to cause mortality upon challenge in chickens. APEC-O78 was serotype O78, APEC-O1 was serotype 01, and APEC O2 was serotype 02. Multiple routes of inoculation were evaluated including intratracheal, intravenous, intravaginal and intraperitoneally in SPF-chickens (Valo BioMedia, Adel, IA)). One isolate designated as APEC-O78 induced repeatable clinical signs of peritonitis when challenged via the intraperitoneal route was selected as the challenge strain for model development of peritonitis.

### Example 5

Preparation of Nalidixic Acid Resistant E. coli Challenge Strain

The E. coli isolates APEC-O78, APEC-01, APEC-O2 and CFTO73 of Example 3 were made nalidixic acid resistant. The importance of inducing resistance to a known antibiotic in the challenge strain is to be able to differentiate the challenge strain from other E. coli strains that may contaminate challenged samples due to its prevalence in the environment. To induce antibiotic resistance each isolate was grown in increasing concentrations of nalidixic acid. Briefly, two 1-liter stock solutions of TSB containing 35 gm Tryptic Soy; 5 gm yeast extract and 2,2-dipyridyl at 25 μg was prepared and autoclaved for 30 minutes at which point was cooled to 4° C. Nalidixic acid was added to one bottle of TSB by membrane filtration through a 0.2 μm filter to a final concentration of 150 μg/mL. The TSB now containing 150 μg nalidixic acid was diluted in 20 mL stocks (50 mL conical tubes) solution using the TSB without nalidixic acid as the diluent to obtain the following concentrations; 0 (no nalidixic acid); 25 μg; 50 μg; 75 μg; 100 μg and non-diluted 150 μg.

The isolates were removed from frozen storage and plated onto sheep blood agar and incubated at 37° C. for 24 hours at which point a single colony was picked and sterilely inoculated into one of the non-nalidixic acid tubes and incubated for 3 hours at 37° C. while stirring at 200 rpm. At three hours post inoculation 2 mL of the culture was transferred into 20 mL of the 25 μg nalidixic acid tubes that was pre-warmed to 37° C. The cultures were allowed to grow at 37° C. while rapidly stirring at 200 rpm for 3 hours. This process was repeated two times and then transferred to the next concentration of nalidixic acid. If growth did not occur the process was repeated in the previous concentration and then transferred to the next increasing concentration. This was done for each concentration until growth was established at the highest concentration of nalidixic acid. Once growth was established at the 150 μg level, the cultures were then plated onto EMB containing 80 μg nalidixic acid. A single colony of each isolate was selected and transferred into 100 mL TSB containing 80 μg/mL. The cultures were allowed to grow at 37° C. for 4.5 hours or until an OD of 1.0 at 540 nm was achieved. The cultures were centrifuged at 8000 rpm for 20 minutes at which point the supernatants were discarded and the pellets re-suspended in 90 mL of TSB media as described above but containing 20% glycerol and 25 μg/mL 2,2-dipyridyl. One mL aliquots of each bacterial suspension were dispensed into 2 mL cryovials and stored at −90° C. until use.

### Example 6

Serial Passage of E. coli in SPF Chickens

To enhance the virulence of the challenge strains O78, 01 and O2 the nalidixic acid resistant isolates were serially passaged in SPF Chickens. Briefly, using the culture as described above, four chickens were intravenously injected with either 0.1 or 0.2 cc at 1.0×10⁹ CFU/mL of the isolate. Twenty-four hours post inoculation chickens were morbid but did not die. Chickens were euthanatized by cervical dislocation and examined for gross pathology lesions characteristic of peritonitis. The liver of each chicken was cultured using a flamed loop and plated onto blood agar and Eosin Methylene Blue (EMB) agar containing 150 μg/mL nalidixic acid. Plates were incubated at 37° C. for 24 hours. A number of colonies from the 0.2 mL dose had grown on both the EMB and blood plate indicating the isolates had gone systemic. These colonies were streaked for isolation and again passed through chickens using the same regiment. This time the 0.2 mL dose had approximately 20-50 colonies. This was repeated; livers cultured, and plates had greater than 100 colonies. The liver cultured isolates were passed two additional times in chickens (3 serial passes total) which resulted in an increase in lesions of the air sacs, pericarditis, perihepatitis and extensive lesions in the oviducts typical of peritonitis. These results clearly demonstrate that the passage of the isolate in the host species (chickens) increased the pathogenesis of the isolate, inducing lesions typically seen as peritonitis.

### Example 7

Serial Passage of E. coli CFTO73 in Mice

To enhance the virulence of the challenge strain CFTO73 the isolate was serially passaged in Harlan CF-1 mice obtained from Charles River Laboratory (Wilmington, MA) weighing 16-22 grams. Briefly, using the cultures as described in Example 3, two mice were subcutaneously injected with either 0.1 or 0.2 mL at 1.0×10⁹ CFU/mL of each isolate. Twenty-four hours post inoculation mice were morbid but did not die. Mice were humanely killed and each liver was cultured using a flamed loop and plated onto blood agar and Eosin Methylene Blue (EMB) agar containing 150 μg nalidixic acid. Plates were incubated at 37° C. for 24 hours. A number of colonies from the 0.2 mL dose had grown on both the EMB and blood plate indicating the isolates had gone systemic. These colonies were streaked for isolation and again passed through mice using the same regiment. This time the 0.2 mL dose had approximately 20-50 colonies. This was repeated; livers cultured and plates had greater than 100 colonies. The liver cultured isolates were passed two additional times in mice (five serial passes total) which resulted in all mice dying at 24 hours post challenge; clearly demonstrating that each of the isolates adapted to grow in the new host species by the enhancement of virulence with death as the outcome parameter.

### Example 8

### Preparation of Frozen Working Seeds

The nalidixic acid resistant E. coli isolates of serial chicken pass three of Example 5 was subcultured from EMB plates and expanded into frozen working seeds. Briefly single colonies from the EMB plates (serial pass five) were subcultured into 20 mL of TSB containing 32 gm TSB; 5 gm yeast extract and 2,2-dipyridyl at 25 μg/liter. The cultures were allowed to stir at 200 rpm for 2 hours at which point were subcultured in the same media that was pre-warmed to 37° C. After the 2 hour time period 10 mL of each culture was transferred to 100 mL of pre-warmed TSB as described above except the concentration of 2,2-dipyridyl was 25 μg/l. These cultures were allowed to grow until they reached an OD 1.0 at 540 nm at which point were centrifuged at 8000 rpm for 10 minutes and re-suspended into 90 mL cold TSB as described above; except it contained 20% glycerol. One mL aliquots of each bacterial suspension was dispensed into 2 mL cryovials; labeled and stored at −90° C. until use.

### Example 9

### Enzyme-Linked Immunosorbent Assay (ELISA)

The serological response to the bacterial extract of E. coli consisting of metal regulated proteins as described in Table 1 was determined by measuring the IgG titers by ELISA. In brief, 100 μl of bacterial extract that included ChuA, IroN, IreA, IutA, FecA, FepA, OmpC, and OmpA was diluted in carbonate-bicarbonate buffer, pH 9.6 at 400 ng per well of a 96-well polystyrene plates (Immunlon 2HB, Thermo-Scientific) 96-well EIA/RIA plate (Corning/Costar 3590) and incubated overnight at 4° C. All remaining steps were performed at room temperature. The plate was washed three times with PBS wash buffer (PBS containing 0.05% Tween 20) and subsequently blocked with 1% polyvinyl acetate in PBS for one hour and re-washed. Serum samples, in duplicate, were diluted 1:1,000 and then four-fold within plates using wash buffer and incubated for 1 hour at 37° C. Plates were then washed and subsequently incubated with goat anti-chicken IgG horse radish peroxidase (KPL, Seracare, USA) for one hour at 37° C. followed by a final wash step. Development of the plates was done using ABTS Substrate System (KPL, Seracare, USA) prior to reading on a Biotek spectrophotometer at 405-490 nm. Gen5 software (Biotek, USA) was used to calculate titers as defined as the point at which a sample's dilution curve intercepted at 50% of the mean optical density value of a positive control sera on the plate. Geometric mean titers and confidence intervals are reported for each treatment group.

### Example 10

### Expression and Purification of Recombinant Proteins

Recombinant proteins cloned from E. coli strain CFT073 were expressed in E. coli and purified using standard methods. In brief, frozen bacterial stocks (100 μl) were used to inoculate 20 mL of Luria-Bertani (LB) Broth containing the appropriate selective antibiotic (kanamycin for pET29b), and the culture was grown at 37° C. in a shaking incubator at 250 rpm. After 16 hours, the culture was diluted into 1 L of LB Broth containing the appropriate selective antibiotic, grown to an optical density (600 nm) of 0.6, and induced by the addition of 1 M IPTG to a final concentration of 1 mM. Alternatively, for larger batches, the culture was used to seed a 10 L fermenter that was induced with IPTG to a final concentration of 1 mM. Bacterial cell pellets were harvested by centrifugation at 4,000×g for 20 minutes at 4° C., washed in PBS, and stored at −80° C. until lysis. Insoluble inclusion bodies were enriched for through treatment with BugBuster (Millipore) and were solubilized in an 8M urea buffer. Anion exchange (AEX) chromatography was utilized to remove endotoxin followed by a second AEX chromatography step to buffer exchange the sample into a final buffer consisting of 20 mM sodium phosphate, 51 mM N-Octyl-β-D-glucopyranoside (nOG), 300 mM sodium chloride, and 300 mM urea (pH 9.5). The protein concentration was determined using the BCA method (Thermo Scientific, Rockford, IL) and protein purity was measured by SDS-PAGE and densitometry (LI-COR Odyssey; LI-COR, Lincoln, NE).

### Example 11

### Large Scale Process for the Manufacture of Metal-Regulated Proteins

Fermentation. A cryogenic vial of the working seed (1 mL at 10⁹ CFU/mL) was used to inoculate 500 mL of 37° C. tryptic soy broth (TSB) without dextrose (Bacto) containing 34 micrograms/liter 2,2-dipyridyl (Sigma), 2.5 grams/liter yeast extract (Bacto) and glycerol (3% vol/vol). The culture was incubated at 37° C. for 16 hours while agitating at 160 rpm, and then divided between two 1.5 L bottles of the above media. This second culture was allowed to grow for an additional 2.5 hours at 37° C. This culture was used to inoculate a 400 L DCI-Biolafitte SIP fermentor, (DCI, St. Cloud, MN) charged with 300 liters of the above-described media with the addition of Mazu DF 204 defoamer (150 mL). The parameters of the fermentation were as follows: dissolved oxygen (DO) was maintained at 60%+/−20% by increasing agitation to 500 rev/minute sparged with 17-120 liters of air/minute, 0-60 liters of air/minute and 5 pounds per square inch (psi) back pressure. The pH was held constant between 6.9 and 7.2 by automatic titration with 50% NaOH and 25% H3PO4. The temperature was maintained at 37° C. The fermentation was allowed to continue growth for 5.5 hours at which point the fermentation was terminated by lowing the temperature of the fermentor to 15° C. and lowering pH to 5.0 with 25% H3PO4 (optical density 15 at 540 nanometers at a 1:20 dilution). The culture was sterilely transferred to a 200-liter tank (LEE Process Systems and Equipment model 2000LDBT) in preparation for harvest.

Harvest. The bacterial fermentation was concentrated and washed using a Pall Filtron Tangential Flow Maxisette-25 (Pall Filtron Corporation, Northboro, MA) equipped with four 30 ft2 Alpha 0.1 um open channel filters (Pall Filtron, catalog No. PSM10C52) connected to a Waukesha Model 130 U2 feed pump (Waukesha Cherry-Burrell, Delevan, WI) The original culture volume of 300 liters was reduced to 60 liters using a filter inlet pressure of 30-40 psi and a retentate pressure of 2-15 psi. The bacterial retentate was then washed using 200 liters of a sodium acetate tryhidrate solution pH 5.0 which was composed of 2.72 grams/liter sodium acetate tryhidrate. The 60 liters of bacterial retentate was then washed with 100 liters of osmotic shock buffer (OMS) containing 14.52 grams/liter Tris-base and 1.86 grams/liter EDTA adjusted to a pH of 8.6. The EDTA in the OMS served to assist removal of much of LPS from the cell wall, while the elevated pH prevented much of the proteolytic degradation after freezing and disruption. Protease inhibitors may be used instead of, or in addition to, an elevated pH. The retentate was then concentrated down to 40 liters to help remove any contaminating exogenous proteins, 200 more liters of the above OMS was then added to wash all bacteria through the filters into the harvest tank. The retentate was mixed thoroughly while in the 200-liter tank using a bottom mount magnetically driven mixer. The retentate was sterilely dispensed (5 liters) into gamma irradiated 5 liter Invitro™ containers and placed into a −20° C. freezer for storage. Freezing the bacterial pellet served to weaken the cell wall structure making downstream disruption more efficient. The pellet mass was calculated by centrifuging 1 mL sample of the fermented culture and final harvest. Pre-weighted 1 mL conical tubes were centrifuged at 13,000 rpms for 10 minutes in a Microfuge 18. The supernatant was poured off and the pellet was re-suspended in sterile water. This mixture was again centrifuged at 13,000 rpms for 5 minutes before it was once again decanted. This washed pellet was placed in a 125° C. oven for 75 minutes before being weighed and extrapolated to determine harvest volume pellet mass. The fermentation process yielded a dry pellet mass of 2.3 kilograms.

Alternative methods for bacterial harvest can be used. Bacterial harvest may be performed by the use of hollow fiber filter methods. Bacterial culture is harvested using filter cartridges ranging in size from 0.2 μM to 5 kDa; preferably with a 750 kDa cartridge. Culture is reduced in volume from 2-20× and subsequently washed 1-5× by diafiltration with buffer prior to storage at 4° C. or freezing at −20° C. In this manner, undesired media proteins, bacterial proteins and LPS are removed from the culture. In another alternative, bacterial harvest may be performed by the use of industrial scale centrifugation, for example, by use of a disc-stack centrifuge.

Disruption (Homogenization). Frozen bacterial cell slurry in OMS were thawed at 4° C. (2.3 kg of pellet mass). The liquid culture suspension from each container was aseptically aspirated into a 200 liter process tank (Model 200LDBT) with a bottom mounted mixer (Lightnin Mixer Model MBI610H55) containing 13 liters OMS pH 8.5. The volume of OMS was determined by calculating the homogenizing volume by multiplying the pellet mass by 30.8 L/Kg and taking the homogenizing volume and subtracting the volume of bacteria from the fermentation harvest. The bulk bacterial suspension was chilled to 4° C. with continuous mixing for 18 hours at 18 Hz at which time it was disrupted by homogenization. Briefly, the 200 liter tank containing the bacterial suspension was connected to an Avestin Model EF-C500B Homogenizer (Avestin, Rosemont, IL). A second 200 liter process tank (empty) was connected to the homogenizer such that the fluid in the process tank could be passed through the homogenizer, into the empty tank and back again, allowing for multiple homogenizing passes while still maintaining a closed system. The temperature during homogenization was kept at 4° C. At the start of each pass, fluid was circulated at 60 psi via a Waukesha model 30U2 pump (Waukesha) through the homogenizer (500 Liters/hour) and back to the tank of origin, while the homogenizer pressure was adjusted to 11,000-30,000 psi. Prior to the first pass, two pre-homogenizing samples were withdrawn from the homogenizer to establish a baseline for determining the degree of disruption and monitoring of pH. The degree of disruption was monitored by transmittance (% T at 540 nm at 1:100 dilution) compared to the non-homogenized sample. The number of passes through the homogenizer was standardized for different organisms based on the integrity of the cell wall and variation in the degree of disruption, which had a direct correlation in the efficiency of solubilization and quality of end product. For example, the disruption of Salmonella passed two times through the homogenizer gave a final percent transmittance between 78-83% T at a 1:100 dilution. E. coli having the same pellet mass and starting OD gave a % T of 80-86% (at a 1:100 dilution) after the second pass. It has been observed that bacteria differ in their cell wall integrity and vary in their capacity of disruption under identical condition. This variation can affect the degree and efficiency of solubilization and recovery of metal regulated proteins. In general, cells were passed through the homogenizer until the transmittance of at least 80% was reached after a minimum of two passes.

After homogenization, sodium lauroyl sarcosinate (Hamptosyl L-30, Chem/Serv) was aseptically added to the homogenized bacterial suspension for solubilization. The amount of sarcosine (30%) added equaled 0.083 times the solubilizing volume, in liters, (solubilizing volume was determined by multiplying the fermentation dry pellet mass by 34.7 L/Kg). The tank was removed from the homogenizer and placed in a 2-7° C. cooler and mixed at 18 Hz for 12-96 hours. This time period was helpful to complete solubilization. It was discovered that increasing the solubilization time in OMS at an elevated pH (8.0-8.5) that metal regulated proteins aggregated together forming large insoluble aggregates that were easily removed by centrifugation. The optimal OD after solubilization was usually between 25-30% T at 540 nm. 12-24 hours prior to protein harvest 0.15% of formalin was added to the final solubilizing volume as a preservative.

Protein Harvest. The aggregated metal regulated proteins within the solubilized process fluid were collected by centrifugation using T-1 Sharples, (Alfa Laval Separations, Warminster, PA). Briefly, the tank of solubilized homogenate was fed into twelve Sharples with a feed rate of 200 mL/minute at 11 psi at a centrifugal speed of 30,000 rpm. The effluent was collected into a second 200 liter process tank through a closed sterile loop allowing for multiple passes through the centrifuges while maintaining a closed system. The temperature during centrifugation was kept at 4° C. The solubilized homogenate was passed up to 12 times across the centrifuges with a feed rate of 150 mL/minute at 21 psi at a centrifugal speed of 50,000 rpm. Protein was collected after the first pass and discarded, at which point the solubilized fluid was concentrated to 1/3 of its original volume. This decrease in volume shortened the process time for passes 2-12. Briefly, the solubilized homogenate tank was connected to a Pall Filtron AT25 Holder, equipped with three 30.1 ft2 screen-channel series Omega 10 kd Maxisette filters (Pall Filtron) connected to a Waukesha Model 130U2 feed pump for concentration. After concentration, centrifugation was continued until the process was completed. Protein was collected after each pass. The protein was collected, resuspended and dispensed into two 8 Liter containers containing Tris-buffer pH 8.5 containing 0.3% formalin (Sigma) as preservative. The containers were placed into a mixer Model Turbula T10B (M.O. Industries, Wippany, New Jersey) and mixed until the protein was re-suspended in the buffer solution.

Diafiltration. The protein suspension was washed by diafiltration at 4° C. to remove any contaminating sarcosine that may have been bound to the protein. The two containers of protein were aspirated into a 200 Liter tank containing 40 mL TBW/g protein harvested of Tris-Buffer pH 8.5 containing 0.3% formalin equipped with a bottom mount Lightnin mixer, Model MBI610H55 mixing at 20 Hz. The process tank was placed in a 33° C. incubator for a minimum of 12 hours for protein inactivation. The process tank was sterilely connected to a Millipore Pellicon Tangential Flow Filter assembly (Millipore Corporation, Bedford, MA), equipped with two 26.9 ft2 screen-channel series Omega 10K Centrasette filter (Pall Filtron) connected to a Waukesha Model 30U2 feed pump. The solution was concentrated down to approximately 35 liters and was re-suspended with 200 liters of Tris-buffer, pH 7.4, containing 0.1% formalin solution. The solution was again concentrated down to approximately 35 liters and re-suspended again with 200 liters of a Tris-buffer, pH 7.4, containing 0.1% formalin solution. The solution was then concentrated down to approximately 35 liters and re-suspended with 80 liters of Tris-buffer, pH 7.4, containing 0.1% formalin solution. The solution was then concentrated by filtration to a target volume of 6.5 times the protein pellet mass. The protein concentrate was aseptically dispensed into sterile 20 liter Nalgene containers and placed into a 33° C. incubator for 12-24 hours for the final antigen inactivation.

This process produced a composition containing metal regulated proteins with a decrease in the amount of LPS and very little to no sarcosine residue. The protein was examined by SDS-PAGE for purity and banding profile, and also examined for bacterial contamination, residual sarcosine and LPS. The banding profile of the finished product showed consistent patterns as examined by electrophoresis. The composition was tested for sarcosine by the use of a modified agar gel diffusion test in which sheep red blood cells (5%) were incorporated into an agar base (1.5%). Wells were cut into the agar and samples of the finished product along with control samples of known concentrations of sarcosine at 0.05, 0.1, 0.2, 0.3, 0.4, 0.5 1.0 and 2.0% were placed into the wells. The gel was incubated at 25° C. for 24 hours and the degree of hemolysis was determined compared to the controls. The process removes the level of detectable sarcosine below 0.05%, which at this concentration showed minimal hemolysis in control samples. The concentration of LPS was examined by a Limulus amebocyte lysate (LAL) test available under the tradename PYROTELL (Associates of Cape Cod, Inc., East Falmouth, MA).

After cell lysis by freezing and homogenization, protein may be harvested by hollow fiber methods. Bacterial lysate is filtered to separate whole cells and large debris from small particulates and soluble protein. This may be accomplished using a range of sizes of hollow fiber cartridges from 0.2 μM to 5 kDa; preferably with a 0.65 μM nominal pore size. In this manner, whole unlysed cells and large debris are retained and possibly concentrated by the filter while protein and small particulates of interest are passed through the filter and collected. Additionally, it may be desirable to wash the retentate from 1-20× with buffer to increase the harvest of proteins of interest.

Subsequent to the primary harvest above, bacterial membranes of the small particulates are solubilized with sarcosine as described above, followed by further fractionation or protein harvest and wash by hollow fiber methods. This serves three functions: the removal of undesired cytosolic proteins, the removal of undesired membrane components including LPS and the hydrophobic aggregation of desired metal-regulated proteins and porin proteins into higher molecular weight forms. After the solubilizing step, the solution is filtered using hollow fiber cartridges ranging in size from 0.2 μM to 5 kDa; preferably with a Laboratory and/or Pilot Scale Ultrafiltration Cartridge (for example, (UFP-750-E-6A) size 6A Ultrafiltration Hollow Fiber Cartridge (63.5 cm L); Polysulfone membrane, optionally having a 750 000 NMWC pore size, GE Healthcare Pittsburgh, PA). This step can also include concentration (2-20×) and diafiltration wash steps (1×-20×) with buffer and ethanol to enhance the removal of undesired protein, membraneous components, DNA and sarcosine and thus increase the purity of the harvested metal-regulated proteins and porin proteins.

An example of the proteins present in the composition prepared as described above is shown in FIG. 1. Six higher molecular weight proteins ChuA, IroN, IreA, IutA, FecA FepA (and two lower molecular weight proteins identified as OmpC and OmpA in lanes 1 and 2) were observed after resolving the proteins on an SDS-PAGE gel.

### Example 12

### Peritonitis Model Development in Pathogen Free White Leghorn Chickens

Studies were undertaken in specific pathogen free white leghorn chickens for the development of a chicken model of avian pathogenic Escherichia coli (APEC) peritonitis and generalized colibacillosis herein. The model was used to measure the efficacy of a composition of siderophore receptor and porin proteins of Example 11. Examples 12-17 are described in Cox et al., 2020, Avian Diseases, 65(1):198-204, https://doi.org/10.1637/aviandiseases-D-20-00093.

Five pilot studies were performed to i) compare the virulence of E. coli serotypes (APEC-01, APEC-O2 and APEC-O78) in their ability to induce lesions of peritonitis, ii) evaluate routes of challenge, and iii) standardize the dose of inoculum that results in pathology characteristic of peritonitis observed in commercial layer facilities such as widespread organ infection, atrophy, discoloration, corrugation of yolk sacs and the presence of caseous exudate.

Preliminary ModelDevelopment. Isolates of serotype O1, O2 and O78 of Example 4 were tested by intravenous (IV), intravaginal (IVAG), intratracheal (IT) and intraperitoneal (IP) routes and were compared at varying levels of challenge inoculum. Daily observations of mortality and morbidity were made, and at necropsy, gross lesion scores were collected, and bacterial colonization of internal organs determined.

Outcomes varied from a complete lack of mortality or detectable pathology and low, or no, organ colonization in the case of IVAG and IT routes with each E. coli serotype to moderate to high levels of mortality, pathology and colonization after challenge via the IV and IP routes with O2 and O78 serotypes, respectively. The O78 serotype was found to result in pathologies consistent with field observations of peritonitis and therefore, subsequent studies were performed only with O78 (Table 2). In addition to the relative failure with both the IT and IVAG routes of challenge, the IV route was found to be inconsistent and often resulted in lameness not observed with the IP route (refer to Table 2). A final pilot study confirmed that the dose (˜8 log 10 CFU) administered by the IP route replicated peritonitis. In addition, the IP route induced lesions of the organs such as airsacculitis, pericarditis, and perihepatitis.

Three studies (Table 3, A, B, C, grouped by serotype) were initially performed to compare O1, O2 and O78 serotypes using IV, IT, and IVAG challenge routes. Our goal was to mimic the development of E. coli peritonitis pathology that had been observed in commercial egg-laying facilities. These studies were largely ineffective with any of the three serotypes using either the IVAG or IT challenge routes and only the IV route resulted in pathology or colonization. Mortality with the O1 serotype was severe at 9 log 10 CFU of challenge and no mortality at 8 log 10 CFU with no observed pathology in all ten chickens. Mortality with the O2 serotype was lower (4/15 chickens) and all but one chicken had pathology within the entire abdominal cavity. All but two chickens were positive for O2 challenge colonization. For the O78 serotype, the IV route at 8 log 10 CFU resulted in no mortality, no observable pathology and only two of ten chickens were colonized. Consistent across the three serotypes was frequent lameness in the IV challenged chickens (data not shown). The lack of peritonitis from either of the IT and IVAG routes of challenge administration eliminated them from further consideration.

In a subsequent study (Table 3, D), the IV route was compared to the IP route using the O78 serotype. Both routes resulted in peritonitis, organ colonization, and low or no mortality. However, once again, the IV route did result in significant lameness (data not shown). Due to this lameness and the interest in establishing a route of challenge more closely replicating a natural infection, the IV route was dropped from further consideration. In the final two studies (Table 3, E, F), challenge dose-ranging was performed with the O78 serotype and by the IP route. Taken together, the studies were characterized by low to moderate mortality, a high incidence of peritonitis, and moderate levels of organ colonization at challenge doses of 7-8 log 10 CFU. Due to the success of these final O78 serotype studies, the same conditions were used in the following vaccination/challenge studies.

TABLE 3
Incidence of mortality, pathology and organ colonization with
different challenge serotype, route and dose administered.
Data are compiled from five separate studies and
grouped (A through F) by serotype, where each
row represents a unique treatment.

| | | Challenge | Challenge | | | |
|---|---|---|---|---|---|---|
| | | Age | Route¹ | | | Organ |
| Challenge | | (wks), | and Titer | Mor- | | Colo- |
| Serotype | | n = | (CFU) | tality² | Pathology³ | nization⁴ |
| A | O1 | 20, n = 10 | IV 8 log₁₀ | 0/10 | 0/10 | nd⁵ |
| | | 21, n = 15 | IV 9 log₁₀ | 14/15 | nd | nd |
| | | 20, n = 10 | IT 9 log₁₀ | 0/10 | 0/10 | nd |
| | | 20, n = 10 | IVAG 9 log₁₀ | 0/10 | 0/10 | nd |
| B | O2 | 24, n = 10 | IV 8 log₁₀ | 0/10 | 0/10 | 2/10 |
| | | 21, n = 15 | IV 9 log₁₀ | 4/15 | 14/15 | 13/15 |
| | | 24, n = 10 | IT 9 log₁₀ | 0/10 | 0/10 | 0/10 |
| | | 24, n = 10 | IVAG 9 log₁₀ | 0/10 | 0/10 | 0/10 |
| C | O78 | 24, n = 10 | IV 8 log₁₀ | 0/10 | 0/10 | 2/10 |
| | | 24, n = 10 | IT 9 log₁₀ | 0/10 | 0/10 | 0/10 |
| | | 24, n = 10 | IVAG 9 log₁₀ | 0/10 | 0/10 | 0/10 |
| D | O78 | 25, n = 10 | IV 9 log₁₀ | 3/10 | 9/10 | 6/10 |
| | | 25, n = 5 | IP 8 log₁₀ | 0/5 | 3/5 | 2/5 |
| E | O78 | 24, n = 4 | IP 8 log₁₀ | 1/4 | 3/3 | nd |
| | | 24, n = 5 | IP 7 log₁₀ | 1/5 | 4/4 | nd |
| | | 24, n = 4 | IP 6 log₁₀ | 0/4 | nd | nd |
| F | O78 | 8, n = 10 | IP 1.5 × 10⁸ | 3/10 | 10/10 | 6/10 |
| | | 8, n = 10 | IP 0.9 × 10⁸ | 4/10 | 9/10 | 7/10 |
| | | 8, n = 10 | IP 0.1 × 10⁸ | 1/10 | nd | nd |

¹Challenge Route: IV intravenous, IT intratracheal, IVAG intravaginal, IP intraperitoneal. Volume of challenge 1 mL.
²Chickens found dead at observation periods or euthanatized due to morbidity. Necropsies were performed on deceased chickens or 7 days after challenge.
³Sum of pathology scores: No pathology = 0, Pathology in any site or organ = 1.
⁴A chicken was considered positive for colonization if any site or organ was positive for challenge culture.
⁵nd. Not done. Example 13

### Chicken Husbandry

Five vaccination/challenge studies were done based on the model development as described above where variables of chicken age, vaccination interval and vaccination to challenge interval were examined. For all studies, Specific Pathogen Free (SPF) White Leghorn chickens or embryonated eggs of the same breed were obtained from Valo BioMedia (Adel, IA) and either hatched at Epitopix facilities and/or reared until enrolled into a study. Chickens enrolled in studies were housed in single rooms within a floor pen containing wood shavings and sunflower seed hulls and were fed and watered ad libitum. Roosts and environmental enrichments were provided, and housing density was lower than that recommended in the USA National Chicken Council Animal Care Guidelines. In the course of all studies, chickens unable to access food or water under their own efforts were humanely euthanatized. Lighting was provided at 12 L:12D throughout each study and the intensity set at 15 LUX. For studies with mature hens older than 17 weeks of age (WOA), lighting was increased to 16 L:8D and 30 LUX to support reproductive organ development and egg laying. Chickens were tagged with numbered wing bands on both wings upon enrollment into a study, randomly allocated, and commingled for the duration of the study. All chickens used in these studies were hens except for Study 4 (Table 4) which was a mixed sex population.

### Example 14

Preparation of the Immunizing Compositions containing metal regulated proteins (ChuA, IroN, IreA, IutA, FecA and FepA)

The metal regulated proteins prepared from E. coli as described in Example 11 were used to prepare compositions for administration to Chickens to determine the efficacy of the vaccine against a live virulent E. coli challenge as established in Example 12. Two separate vaccine formulations were prepared using either a water-in-oil emulsion using light mineral oil and/or formulated in aluminum hydroxide (AlOH).

The water-in-oil emulsion was prepared using the following constituents; 44.44% aqueous protein suspension (standardized to 100 μg total protein per chicken dose, 50 μg derived from 1966 and 50 μg derived from 1967), 50% Drakeol 6 mineral oil (VOPAK USA, Inc, Kirkland, Wash.), 3.0% Span 85 and 2.56% Tween 85 (Ruger Chemicals, Hillside, N.J.). The constituents were combined and dispensed into a vessel equipped with a high-speed emulsifier (IKA model Process pilot 2000/4 or equivalent). The emulsifier was set at 60 hz, and the aqueous solution was pumped into the oil, which was pre-cooled to 4° C. The vaccine was continuously stirred as it was pumped into sterile high-density polyethylene bottles using silicone stoppers and aluminum seals. The bottled vaccine was stored at 4° C. until use. The Vaccine using aluminum hydroxide (AlOH) was formulated at 250 μg dose levels of total protein as described above in phosphate buffered saline (PBS) containing 8.0 g/l NaCl, 0.2 g/l KCl, 1.44 g/l Na2HPO4 and 0.24 g/l KH2PO4 pH 7.4 formulated with 25 percent (v/v) Rehydragel HPA; (General Chemical; Berkeley Heights; New Jersey). Briefly, the antigen/aluminum hydroxide suspensions were stirred for 24 hours at 4° C. to allow maximum adsorption of the protein to the adjuvant in a final volume of 0.25 mL. The final vaccine was aliquoted into sterile high density 500 mL polyethylene bottles using silicone stoppers and aluminum seals and stored at 4° C. until use. Placebo vaccines for each formulation as described above was prepared by substituting physiological saline for the aqueous protein suspension. The placebo vaccines were bottled in 500 mL polyethylene bottles as described above and stored at 4° C. until use.

All vaccines and challenge regiments were done where variables of chicken age, vaccination interval, and vaccination to challenge interval were examined. Farm and laboratory staff were blind to all challenge and vaccine treatments, and observations and samples were identified only by wing tag number. Investigators were similarly blind to treatments and study groups except for the last two studies listed in Table 3, (E and F). Chickens were vaccinated subcutaneously in the back of the neck with 0.25 mL of vaccine unless otherwise specified. Blood was taken two weeks after first or second vaccination from the wing vein of chickens into SST tubes (Becton, Dickinson and Co., Franklin Lakes, NJ) and allowed to clot at ambient temperature for four hours. Sera was then collected by centrifugation and subsequently stored at −20° C. until analyzed. The serological response to vaccination was measured by ELISA of Example 9.

Statistical methods. Analyses of mortality, colonization and pathology data were performed by Fisher's Exact Test using Graphpad Prism (Graphpad Software, Inc., La Jolla, CA). P-values <0.05 were considered statistically significant. Antibody titers between treatment groups were analyzed by ANOVA of log-transformed titer values and Tukey's Honest Significant Differences to assess which groups differed from each other.

### Example 15

### Vaccination and Challenge

Three studies were conducted in SPF White Leghorn chickens looking at the age of vaccination and interval of vaccination. Studies were designated as Study-1, Study-2 and Study-3 (Table 4, column 1); evaluating the efficacy of the vaccine on mortality, pathology and clearance of the challenge organism in the air-sac, liver, heart spleen, oviduct and ovary compared to placebo vaccinated controls. Study-1 received their first vaccination at 28 weeks of age and boosted three weeks later and challenged 2 weeks after second vaccination (28, 31, 33) whereas Study 2 and 3 were vaccinated at 10 and 18 weeks of age, and 4 and 6 weeks of age, respectively, and subsequently challenged 2 weeks later (Table 4, column 4).

All chickens were IP challenged using a 1 mL syringe with a 1″ 23-gauge needle and injection between the cloaca and tip of the keel bone into the peritoneal cavity. The challenge dose was 1.0×10⁸ CFU in a volume of 1 mL.

Necropsy was performed on all chickens after death or seven days after challenge. Chickens unable to access feed and water and considered moribund and were euthanatized to alleviate pain and suffering. These chickens were counted as mortalities for the purpose of statistical analyses. At the time of necropsy, observations of tissue pathology were made and recorded as either positive or negative for some or all of the following tissues: heart, liver, spleen, air sacs and, for mature hens, oviduct and ovary. Specific pathologies included in data capture encompassed caseous exudates to include accumulation of fibrinous material around the heart (pericarditis), liver (perihepatitis), yellowish caseous exudate in subcutaneous tissue (cellulitis), inside the abdominal cavity (peritonitis), caseous exudate in oviduct (salpingitis), necrotic or petechial lesions, off-colors and hyperemia, adhesions, yolk sac fusion, atrophy and cloudiness. Tissues were sampled and tested for the presence of the challenge organism by plating on eosin-methylene blue containing nalidixic acid (80 ug/mL) agar plates. Pathology and colonization are separate events. Sites positive for pathology were observed that were free of the challenge organism, and colonized sites were observed that were negative for pathology.

### Example 16

Preparation of E. coli O78 Challenge Organism

The E. coli O78 isolate as previously described in Example 4 was used for the challenge. Briefly, the isolate, from a frozen stock of Example 8, was subcultured into 100 mL of tryptic soy broth (Difco) containing 25 μg per mL of 2,2′ dipyridyl (Sigma) and 80 μg/mL nalidixic acid. The culture was allowed to grow for 12 hours at 37° C. while rotating at 200 rpm, at which point the culture was subcultured 1:10 into a final volume of 100 mL of tryptic soy broth as described above. The culture was incubated at 37° C. for 2-3 hours while rotating at 200 rpm and allowed to reach an optical density (540 nm) of 0.9-1.3. The cultures were then centrifuged at 8,000×g for 15 minutes at 4° C. to pellet the bacteria. The bacterial pellet was washed twice with cold PBS held at 4° C. and final dilutions made in PBS to achieve ˜1×10⁸ CFU/mL. Just after challenge, the final bacterial suspension was serially diluted and plated onto blood agar and eosin methylene blue (EMB) agar (containing 80 μg per mL of Nalidixic acid) to enumerate the number of colony-forming units (CFU) per dose.

### Example 17

### Results

Five vaccine studies are summarized here that demonstrate the efficacy and performance of the immunizing composition containing the siderophore receptor proteins ChuA, IroN, IreA, IutA, FecA and FepA (Table 4). In each study, the effectiveness of the SRP-E. coli vaccine was dramatic in the face of a severe E. coli challenge. Across all the vaccinated groups the vaccine prevented septicemia, shown as 100% protection, no mortality occurred, compared to incidence rates of 40% to 85% in placebo vaccinated control chickens (Study 1-5). Similarly, pathology rates in vaccinates were completely absent or much reduced (0% to 24%) compared to control chickens (59% to 100%) (Study 1-4). Consistent with these data, the incidence of colonization of vaccinates was greatly reduced (0% to 40%) compared to placebo treated chickens (57% to 88%) (Study 1-3). In every case of comparison of vaccinates to control chickens, the groups were significantly different from each other (p<0.05).

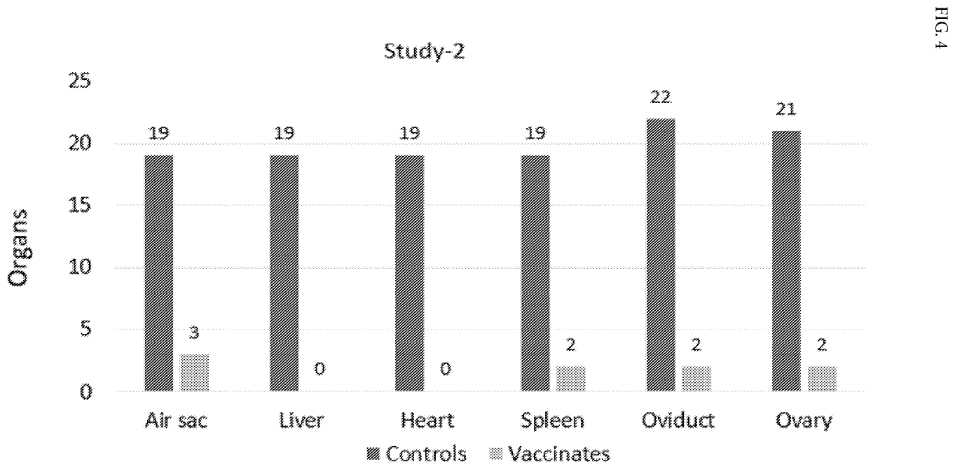

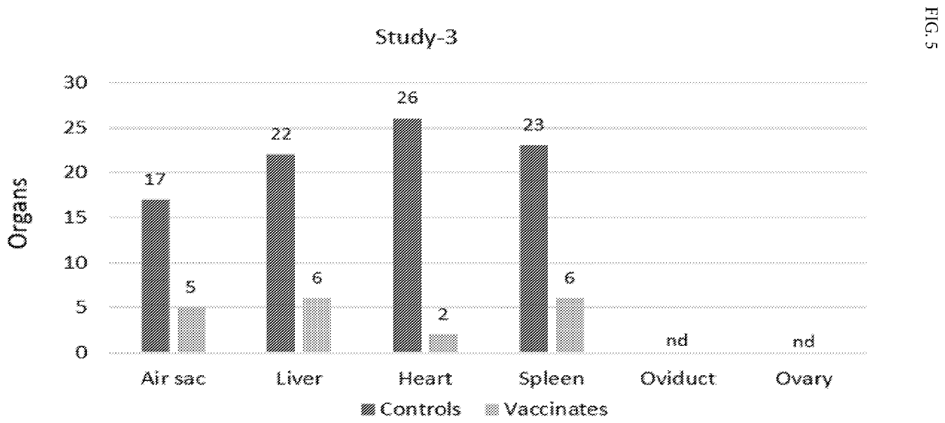

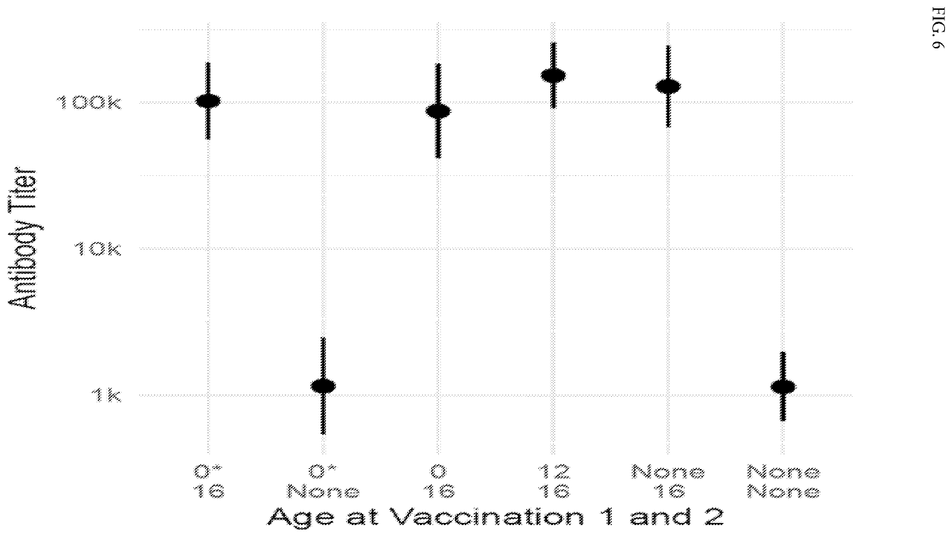

Specific interests within the vaccine studies themselves included an understanding of the onset of immunity, the duration of immunity and the possible influence of the age of chickens, as well as vaccination-challenge intervals on study outcomes. Study-1 was undertaken as the first comprehensive test of the model itself when the chickens were in peak egg production and when stress of this condition may be thought to interfere with the development of immunity sufficient to protect against this severe challenge. Here, the vaccination interval of 3 weeks was typical of many commercial operations and it is clearly demonstrated that the onset of immunity is rapid and robust, with complete protection from mortality two weeks after booster vaccination. Consistent with these data, the incidence of pathology and organ colonization were significantly reduced in vaccinated chickens (Table 4, Study-1.) Study-3 was conducted in younger aged chickens, and the onset of immunity is similar in that complete protection from mortality was observed 2 weeks after booster vaccination. Furthermore, Study-3 indicates that a narrow vaccination interval of 2 weeks is highly effective in protecting young pullets from mortality, colonization and the development of tissue pathology (Table 4, Study-3). Regardless of the treatment, age, and/or vaccination interval the results in studies 1, 2 and 3 were consistent in decreasing mortality and decreasing colonization of organs after an E. coli challenge. FIG. 2 and Table 43 show the mortality in studies 1, 2 and 3 between non-vaccinated controls and vaccinates. Birds in study-1 received their first vaccination at 28 weeks of age and boosted at three weeks post and challenged 3 weeks after second vaccination. No mortality was observed in the birds that were vaccinated in contrast to the non-vaccinated controls showing 8 out of 15 birds died after challenge. These results were repeated in studies 2 and 3 in different age groups of birds with mortality of 19 out of 25 and 12 out of 30 respectively (FIG. 2). No mortality was observed in any of the vaccinated birds in either group.

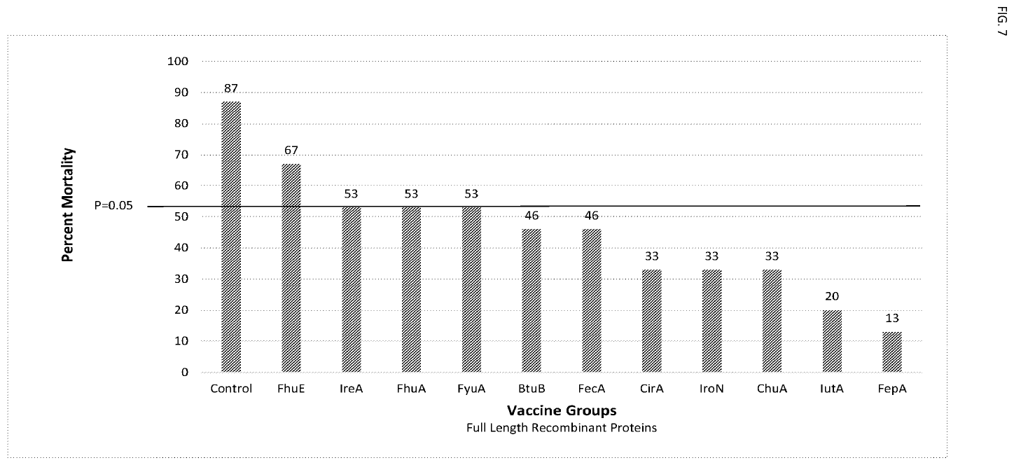

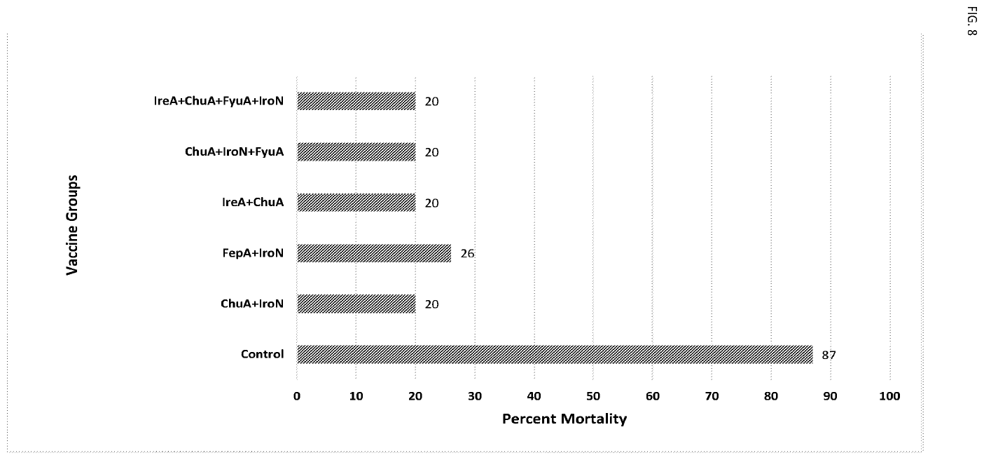

In addition, vaccination decreased the colonization or prevalence of E. coli in organs after challenge (FIGS. 3, 4, and 5). The results clearly show the difference in the efficacy of reducing the incidence of E. coli between vaccinates and non-vaccinated controls. In all organs tested, the results showed statistically significant protection over the non-vaccinated controls. These results clearly demonstrate the efficacy of the vaccine in preventing E. coli septicemia that often leads to generalized infection of the organs, oviduct and ovary.

Following up on vaccination interval as a variable, it was of interest to understand the influence of a broad application window on vaccine effectiveness. The interval in Study-2 (8 wks) is consistent with current autogenous SRP vaccine use in certain commercial pullet rearing facilities and was demonstrated here to be highly effective against mortality, colonization, and the development of peritonitis (Table 4, Study-2). The vaccination interval in Study-4 (16 wks) and Study-5 (15 wks) resulted in similarly effective protection against E. coli challenge (Table 4, Study4 and Study-5).

In addition to the broad interval applied in Study-4, three groups of chickens were vaccinated at day-of-age (DOA) (indicated by Week 0 in Table 4, Study-4) with vaccine adjuvanted with either AlOH or water-in-oil emulsion and their serologic responses measured. A vigorous antibody response was demonstrated by ELISA in all groups that received two vaccinations, regardless of the vaccination interval or age of the chickens (FIG. 6). Geometric mean titers across the responding groups were not significantly different from each other and ranged from ˜87,000 to ˜150,000 (ANOVA on log-transformed titer values; p<0.00000001; Tukey's Honest Significant Difference showed groups 2 and 6 as different from groups 1, 3, 4, and 5 (all adjusted p-values <0.000001), but no groups were different from each other within those clusters (all adjusted p-values>0.8). Consistent with the antibody titers and challenge outcomes of the untreated chickens, those chickens that were vaccinated once at day-of-age with the AlOH adjuvant did not have antibody titers above assay background 18 weeks later (FIG. 6, (1, None) and (None, None)), nor were they protected from challenge (Table 4, Study-4). Interestingly, chickens vaccinated once with standard water-in-oil adjuvant at 16 weeks-of-age had serum IgG titers indistinguishable from all twice-vaccinated chickens (FIG. 6, (None, 16)) and were completely protected from mortality and pathology (Table 4, Study-4) two weeks after vaccination. The duration of immunity (DOI) was examined for two doses of vaccine in Study-5. Here chickens were challenged 12 weeks after second vaccination and mortality in vaccinates was nil compared to 46% mortality in the placebo-treated chickens (Table 4, Study-5). Further DOI data from once- or twice-vaccinated flocks are currently being collected from field studies and will be reported elsewhere.

In all studies, the vaccine demonstrated a high degree of efficacy against mortality, showing a 100 percent protection against challenge. In addition, the vaccine dramatically reduced tissue colonization and pathology typical of APEC infections. The vaccine elicited a rapid onset of immunity with both narrow and broad vaccination intervals and in both young and mature chickens. Additionally, the vaccine was demonstrated to sustain robust effectiveness against mortality over a three-month duration. The use of this vaccine under commercial rearing conditions will provide effective protection of young and mature chickens from pathogenic E. coli strains under broadly flexible conditions of use.

TABLE 4
Incidence of mortality, pathology and organ colonization compared by treatment, age, and vaccination interval.

| | | | Age at V1, | | | Colonization | | | | | |
|---|---|---|---|---|---|---|---|---|---|---|---|
| | | | V2 and | | | Air | | | | | |
| Study¹ | Groups | n | CH (wks)² | Mortality³ | Pathology⁴ | Sac | Liver | Heart | Spleen | Oviduct | Ovary |
| 1 | Placebo | 15 | 28, 31, 33 | 8/15^{a} | 13/15^{a} | nd⁵ | 11/15^{a} | 12/15^{a} | nd | 13/15^{a} | 14/15^{a} |
| | Vaccinates | | | 0/15^{b} | 3/15^{b} | nd | 3/15^{b} | 4/15^{b} | nd | 6/15^{b} | 4/15^{b} |
| 2 | Placebo | 25 | 10, 18, 20 | 19/25^{a} | 129/150^{a} | 19/25^{a} | 19/25^{a} | 19/25^{a} | 19/25^{a} | 22/25^{a} | 21/25^{a} |
| | Vaccinates | | | 0/25^{b} | 36/150^{b} | 3/25^{b} | 0/25^{b} | 0/25^{b} | 2/25^{b} | 2/25^{b} | 2/25^{b} |
| 3 | Placebo | 30 | 4, 6, 8 | 12/30^{a} | 71/120^{a} | 17/30^{a} | 22/30^{a} | 26/30^{a} | 23/30^{a} | nd | nd |
| | Vaccinates | | | 0/30^{b} | 25/120^{b} | 5/30^{b} | 6/30^{b} | 2/30^{b} | 6/30^{b} | nd | nd |
| 4 | V1- AlOH⁶ | 15 | 0, 16, 20 | 0/15^{b} | 0/15^{b} | nd | nd | nd | nd | nd | nd |
| | V1-AlOH | 15 | 0, no V2, 20 | 10/15^{a} | 15/15^{a} | nd | nd | nd | nd | nd | nd |
| | V1 - WIO⁷ | 14 | 0, 16,20 | 0/14^{b} | 0/14^{b} | nd | nd | nd | nd | nd | nd |
| | V1V2WIO | 15 | 12, 16, 20 | 0/15^{b} | 0/15^{b} | nd | nd | nd | nd | nd | nd |
| | V1 - WIO | 21 | 16, no V2, 20 | 0/21^{b} | 0/21^{b} | nd | nd | nd | nd | nd | nd |
| | Untreated | 21 | No V1 or | 18/21^{a} | 19/21^{a} | nd | nd | nd | nd | nd | nd |
| | | | V2, 20 | | | | | | | | |
| 5 | Placebo | 35 | 10, 25, 36 | 16/35^{a} | nd | nd | nd | nd | nd | nd | nd |
| | Vaccinates | 34 | | 0/34^{b} | nd | nd | nd | nd | nd | nd | nd |

¹Within each study, column values with different letter superscripts are significantly different from each other (Fisher's Exact Test, p < 0.05).
²V1—first vaccination, V2—second vaccination, CH—challenge. All vaccine was 0.25 mL/dose and water-in-oil (WIO)-adjuvanted, unless otherwise indicated, and injected SQ in the back of the neck. Placebo contained all components of vaccine except antigen. Challenge for all studies was ~1 × 10⁸ CFU in 1 mL injected IP. V1 of 0 for three groups in Study 4 was done at day of age.
³Chickens found dead at observation periods or euthanatized due to morbidity. Necropsies were performed on deceased chickens or otherwise on euthanatized chickens seven days after challenge.
⁴Sum of Pathology scores: No pathology = 0, Pathology in any tissue or organ = 1. Studies 2 and 3 reflect aggregate pathology scores for all tissues examined.
⁵nd. Not done.
⁶V1 dose volume was reduced to 0.2 mL and the adjuvant used was aluminum hydroxide.
⁷V1 dose volume was reduced to 0.1 mL and the adjuvant used was the water-in-oil adjuvant.

### Example 18

Evaluation of recombinant siderophore receptor proteins in protecting against an E. coli challenge

In this study, we evaluated the efficacy of individual recombinant proteins that were identified by MALDI of Example 2 and two other proteins. Mass spectrometry analyses of trypsin fragments of gel-isolated siderophore receptor proteins from these isolates collectively identified the following proteins: ChuA, IroN, IreA, IutA, FecA and FepA. Genomic analyses by PCR indicated that genes for other siderophore receptor proteins (FhuE, Fiu, FhuA, CirA, FyuA and BtuB) exist in these isolates, but it is unknown if they are expressed during growth under iron-restricted conditions. The recombinant proteins were IutA, ChuA, IroN, FepA, and IreA, and were cloned from CFT073 as described in Example 10 (see FIG. 9). Two additional recombinant proteins, BtuB and CirA, were also cloned from CFT073 as described in Example 10 (see FIG. 9). Upon genomic profiling of iron-responsive genes in CFT073 it was shown that this isolate did not have the gene for FecA. Thus, the gene sequence for this protein was derived from Klebsiella and the expressed recombinant protein was used in this study (see FIG. 9). The FecA protein encoded by the Klebsiella-derived coding region has 98.7% identity to the E. coli-derived FecA protein at SEQ ID NO:16. The recombinant proteins IutA, ChuA, IroN, FepA, IreA, and CirA were expressed with the N-terminal His tag MRGSHIIHHHHHGS (SEQ ID NO:46). The recombinant proteins BtuB and FecA were expressed with the N-terminal sequence of MRGSHHHIHHHGSGSGSGIEGRP (SEQ ID NO:47), which includes an N-terminal His tag, an IE/GR recognition site for Factor Xa, and additional amino acids encoded by vector sequences. In addition, the following formulations consisting of multiple combinations of recombinant proteins: IreA+ChuA, FepA+IroN, ChuA+IroN, ChuA+IroN+FyuA and IreA+ChuA+FyuA+IroN were also examined. The primary outcome parameter used to evaluate vaccine efficacy was to i) evaluate the efficacy of each individual recombinant protein, against an E. coli challenge with strain CFT073 in a murine sepsis model and ii) determine the efficacy of combined recombinant proteins in single vaccine formulations.

Briefly, 255 female Harlan CF-1 mice obtained from Charles River Laboratory (Wilmington, MA) weighing 16-22 grams were equally divided into 17 experimental groups (15 mice/group) designated as 1-17 (Table 5). Mice in groups 2-17 were vaccinated subcutaneously twice at a 21-day interval with a volume of 100 ul of the appropriate vaccine formulation containing single and/or multiple recombinant protein and challenged 21 days post second vaccination (Table 5). Mice in group 1 acted as the placebo vaccinated controls. The placebo vaccine of group 1 was prepared by substituting PBS for the aqueous protein suspension. Mice were housed in polycarbonate cages (Ancore Corporation, Bellmore, N.Y.) at 5 mice per cage with food and water supplied ad libitum. All mice were allowed to acclimate one week prior to the first vaccination.

TABLE 5
Experimental Design.

| | | | Total |
|---|---|---|---|
| Group | Mice | Vaccine | Antigen (μg) |
| 1 | 15 | Control | N/A |
| 2 | 15 | FhuE | 100 μg |
| 3 | 15 | IreA | 100 μg |
| 4 | 15 | FhuA | 100 μg |
| 5 | 15 | FyuA | 100 μg |
| 6 | 15 | *FecA | 100 μg |
| 7 | 15 | CirA | 100 μg |
| 8 | 15 | IroN | 100 μg |
| 9 | 15 | ChuA | 100 μg |
| 10 | 15 | IutA | 100 μg |
| 11 | 15 | FepA | 100 μg |
| 12 | 15 | BtuB | 100 μg |
| 13 | 15 | IreA + ChuA + FyuA + IroN | 400 μg |
| 14 | 15 | ChuA + IroN + FyuA | 300 μg |
| 15 | 15 | IreA + ChuA | 200 μg |
| 16 | 15 | FepA + IroN | 200 μg |
| 17 | 15 | ChuA + IroN | 200 μg |

Mice were divided into 17 experimental groups (15 mice/group) designated as 1-17 and vaccinated two times at a 21-day interval. Mice were challenged 21 days after second vaccination with E. coli CFTO73. Mice in group six were vaccinated with *FecA derived from Klebsiella.

### Example 19

### Vaccine Preparation and Vaccination

Vaccines containing the single and multiple recombinant proteins were prepared at 100 μg dose level of each protein in phosphate buffered saline (PBS) containing 8.0 g/l NaCl, 0.2 g/l KCl, 1.44 g/l Na2HPO4 and 0.24 g/l KH2PO4 pH 7.4 formulated with 20 percent (v/v) Alhydrogel (Invivogen, San Diego, CA) in a final injectable volume of 0.1 mL (Table 5). The placebo vaccines were prepared by substituting PBS for the aqueous protein suspension. Mice were vaccinated twice at a 21-day interval.

### Example 20

### Preparation of Challenge Organism

The E. coli CFTO73 isolate previously described (Example 7) was used as the challenge strain. This isolate was shown previously to express a large repertoire of siderophore receptor proteins. Briefly, the isolate from a frozen stock of Example 4 was streaked onto a blood agar plate and incubated at 37° C. 18 hours. A single colony was subcultured into 50 mL of Tryptic Soy Broth (Difco) containing 25 μg per mL of 2,2′ dipyridyl (Sigma). The cultures were allowed to grow for 12 hours at 37° C. while rotating at 200 rpm, at which point was subcultured 1:100 into a final volume 50 mL of Tryptic Soy Broth as described above. The cultures were incubated at 37° C. for 3 hours while rotating at 200 rpm and allowed to reach an optical density (540 nm) of 0.6-0.8. The culture was then subcultured a final time by transferring 10 mL into 90 mL of pre-warmed TSB containing 25 μg per mL of 2,2′ dipyridyl (Sigma) and incubated at 37° C. for approximately 4 hours while rotating at 200 rpm until an optical density of 0.90-0.95 at 540 nm was reached. The growth of each culture was stopped by adding 100 mL of PBS at 4° C. The cultures were then centrifuged at 10,000×g for 15 minutes at 4° C. to pellet the bacteria. The bacterial pellet was washed once by centrifugation in PBS at 4° C. Twenty-five milliliters of cold PBS was added to each bacterial pellet and vortexed vigorously for 30 seconds to thoroughly resuspend the pellets. This was followed by 75 mL of cold PBS added to the bacterial suspension for a final volume of 100 mL. The culture was then diluted 1:2 in cold PBS and used for challenge. Just prior to challenge, 1.0 mL of the final bacterial suspension was serially diluted 10-fold and plated onto blood agar and eosin methylene blue (EMB) agar (containing 100 μg per mL of nalidixic acid) to enumerate the number of colony-forming units (CFU) per mouse dose.

### Example 21

### Challenge

Twenty-one days after the second vaccination, all mice in groups 1-17 were subcutaneously challenged with 0.1 mL at 1.0×10⁵ colony forming units (CFU) of the virulent E. coli challenge isolate to evaluate the protective efficacy of each recombinant protein in single and multiple protein formulations. Mortality was recorded daily for 7 days post-challenge. Table 6 shows the daily mortality and total percent mortality of all groups following challenge with E. coli for the 7-day observation period.

TABLE 6
Daily mortality of all groups following an E. coli challenge

| | | | Day | Day | Day | Day | Day | Day | Day | Percent |
|---|---|---|---|---|---|---|---|---|---|---|
| Groups | Vaccine | Challenge | 1 | 2 | 3 | 4 | 5 | 6 | 7 | Mortality |
| 1 | Control/Non Vac/Chlg | 0 | 5 | 5 | 2 | 0 | 1 | 0 | 0 | 87 |
| 2 | FhuE | 0 | 0 | 3 | 5 | 1 | 1 | 0 | 0 | 67 |
| 3 | Ire A | 0 | 0 | 1 | 4 | 2 | 0 | 1 | 1 | 53 |
| 4 | FhuA | 0 | 0 | 3 | 4 | 1 | 0 | 0 | 0 | 53 |
| 5 | FyuA | 0 | | 5 | 2 | 1 | 0 | 0 | 0 | 53 |
| 6 | FecA | 0 | 0 | 3 | 1 | 3 | 0 | 0 | 0 | 46* |
| 7 | CirA | 0 | 0 | 1 | 2 | 2 | 0 | 0 | 0 | 33* |
| 8 | IroN | 0 | 0 | 2 | 1 | 2 | 0 | 0 | 0 | 33* |
| 9 | ChuA | 0 | 0 | 2 | 2 | 1 | 0 | 0 | 0 | 33* |
| 10 | IutA | 0 | 0 | 1 | 2 | 0 | 0 | 0 | 0 | 20* |
| 11 | FepA | 0 | 0 | 1 | 1 | 0 | 0 | 0 | 0 | 13* |
| 12 | BtuB | 0 | 0 | 3 | 1 | 3 | 0 | 0 | 0 | 46* |
| 13 | IreA + ChuA + FyuA + IroN | 0 | 0 | 2 | 0 | 1 | 0 | 0 | 0 | 20* |
| 14 | ChuA + IroN + FyuA | 0 | 0 | 1 | 1 | 1 | 0 | 0 | 0 | 20* |
| 15 | IreA + ChuA | 0 | 0 | 0 | 1 | 1 | 1 | 0 | 0 | 20* |
| 16 | FepA + IroN | 0 | 0 | 1 | 0 | 2 | 0 | 1 | 1 | 26* |
| 17 | ChuA + IroN | 0 | 0 | 0 | 2 | 1 | 0 | 0 | 0 | 20* |

Table 6 shows the daily mortality of all groups following an E. coli CFTO73 challenge. Mice were vaccinated two times at 21-day interval and challenged 21 days after the second vaccination. Note the difference in mortality between vaccinates and placebo controls shown as the total Percent Mortality. Groups with an asterisk were statistically significant as compared to non-vaccinated controls.

### Example 22

### Challenge Results

The results showed that 7 out of the 11 recombinant proteins evaluated; showed a high degree of efficacy following challenge. The results showed statistically significant protection over the placebo controls for BtuB, FecA, CirA, IronN, ChuA, IutA and FepA. In contrast, the following proteins, FhuE, IreA, FhuA and FyuA did not reach statistical significance in protection as measured by mortality when compared to the non-vaccinated/challenged controls (Table 6). Interestingly, five out of the six proteins (FecA, CirA, IronN, ChuA, IutA and FepA) have been identified in the peritonitis composition of Example 14 and were highly efficacious against an E. coli systemic challenge showing a high degree of statistical significance. The IreA protein was the only protein identified in the peritonitis composition that did not show protection against challenge in the murine sepsis model, as a single protein. The remaining three proteins (FhuE, FhuA and FyuA) in which the gene sequence was identified in the genomic profile of strains 1966 and 1967 also provided no protection (Table 6, FIG. 7).

All of the recombinant proteins formulated into combination vaccines to include IreA+ChuA; FepA+IroN; ChuA+IroN, ChuA+IroN+FyuA and IreA+ChuA+FyuA+IroN were highly protective against challenge with a degree of significance of p<0.05 (FIG. 8). It is interesting to note that two proteins, the ChuA and IroN proteins, were common in each combination vaccine that showed protection against challenge.

In addition, we chose the E. coli CFT073 isolate for use in this challenge model based on its large repertoire of iron-regulated protein expression and high virulence in mice. It has also been shown that there is a correlation of virulence and certain genes shared between APEC and UPEC isolates. Because avian pathogenic E. coli (APEC) and human uropathogenic E. coli (UPEC) may encounter similar challenges when establishing infection in extraintestinal locations, they share a similar content of virulence genes and capacity to cause disease (Kylie et al., 2005, Microbiology, 151(Pt 6):2097-110). The ability of APEC to acquire and metabolize iron, which enhances growth and virulence, has been extensively documented and is due to proteins encoded by several operons found on large plasmids within APEC isolates. Many of these proteins are shared with UPEC isolates that similarly enhance virulence.

### Example 22

The analysis of Genbank genome sequences of E. coli against 1966 and 1967 siderophore receptor proteins

### Data Overview

The selected genome sequences representing FepA, IroN, IreA, ChuA, FecA and IutA proteins as found in FIGS. 9-1 thru 9-14 were searched against 21,213 published genomes. Briefly, in view of the high level of identity between E. coli FepA proteins, between E. coli IroN proteins, between E. coli IreA proteins, between E. coli ChuA proteins, between E. coli FecA proteins, and between E. coli IutA proteins, hits were limited to those with e-values of less than 0.0000001 and with greater than 90% identity. Further, to avoid genes being counted as hits to multiple genes, the search was limited each to a gene from a genome to its best hit (highest bit score after filters). It is important to note that there was no available data expressing the source of the isolates examined and isolates are assumed to be sourced from different animal species as well as closely related E. coli isolates.

The results showed that of the 21,213 genomes searched, at least 99.5% of the 21, 213 E. coli genomes have at least one of the six siderophore receptor proteins present in the 1966 and 1967 vaccine composition consisting of ChuA, FecA, FepA, IroN, IreA, and IutA. These results were based on a BLAST search of the selected proteins from each genome confirming the presence of a given SRP in the BLAST search; alignment was over 90% and there was not a higher alignment to an additional SRP in the search.

Taken together these results along with the results of example 18 show the importance of these proteins as a vaccine composition and the broad spectrum protection against clinical conditions in multiple animal species the vaccine has against different sero-groups of E. coli.

### CITATIONS

1. Landman W J, van Eck J H. The incidence and economic impact of the Escherichia coli peritonitis syndrome in Dutch poultry farming. Avian Pathol. 44:370-378; 2015.
2. Nolan L K, Barnes H J, Vaillancourt J-P, Abdul-Aziz T, Logue C M. Colibacillosis. In: Swayne D, editor. Diseases of poultry. 13th ed. Ames (IA): Wiley-Blackwell. p. 751-805; 2013.
3. Zanella A, Alborali G L, Bardotti M, Candotti P, Guadagnini P F, Martino P A, Stonfer M. Severe Escherichia coli O111 septicaemia and polyserositis in hens at the start of lay. Avian Pathol. 29:311-317; 2000.
4. Landman W J, Heuvelink A, van Eck J H. Reproduction of the Escherichia coli peritonitis syndrome in laying hens. Avian Pathol. 42:157-162; 2013.
5. Chaudhari A A, Kariyawasam S. An experimental infection model for Escherichia coli egg peritonitis in layer chickens. Avian Dis. 58:25-33; 2014.
6. Huja S, Oren Y, Trost E, Brzuszkiewicz E, Biran D, Blom J, Goesmann A, Gottschalk G, Hacker J, Ron E Z, et al. Genomic avenue to avian colisepticemia. mBio. 6:1-13; 2015.
7. Fernandes Filho T, Favaro C Jr, Ingberman M, Beirao B C, Inoue A, Gomes L, Caron L F. Effect of spray Escherichia coli vaccine on the immunity of poultry. Avian Dis. 57:671-676; 2013.
8. Ghunaim H, Abu-Madi M A, Kariyawasam S. Advances in vaccination against avian pathogenic Escherichia coli respiratory disease: potentials and limitations. Vet Microbiol. 172:13-22; 2014.
9. Gregersen R H, Christensen H, Ewers C, Bisgaard M. Impact of Escherichia coli vaccine on parent stock mortality, first week mortality of broilers and population diversity of E. coli in vaccinated flocks. Avian Pathol. 39:287-295; 2010.
10. La Ragione R M, Woodward M J, Kumar M, Rodenberg J, Fan H, Wales A D, Karaca K. Efficacy of a live attenuated Escherichia coli O78:K80 vaccine in chickens and turkeys. Avian Dis. 57:273-279; 2013.
11. Landman W J M, van Eck J H H. The efficacy of inactivated Escherichia coli autogenous vaccines against the E. coli peritonitis syndrome in layers. Avian Pathol. 46:658-665; 2017.
12. Li L, Thofner I, Christensen J P, Ronco T, Pedersen K, Olsen R H. Evaluation of the efficacy of an autogenous Escherichia coli vaccine in broiler breeders. Avian Pathol. 46:300-308; 2017.
13. Sadeyen J R, Wu Z, Davies H, van Diemen P M, Milicic A, La Ragione R M, Kaiser P, Stevens M P, Dziva F. Immune responses associated with homologous protection conferred by commercial vaccines for control of avian pathogenic Escherichia coli in turkeys. Vet Res. 46:5-14; 2015.
14. Bullen J, Griffiths, E. Iron binding and host defense. In: Bullen J, Griffiths E, editor. Iron and infection: molecular, physiological and clinical aspects, 2nd edition. New York (NY). John Wiley & Sons Inc. p. 327-368; 1999.
15. Hood M I, Skaar E P. Nutritional immunity: transition metals at the pathogen-host interface. Nat Rev Microbiol. 10:525-537; 2012.
16. Miethke M, Marahiel M A. Siderophore-based iron acquisition and pathogen control. Microbiol Mol Biol Rev. 71:413-451; 2007.
17. Caza M, Lepine F, Dozois C M. Secretion, but not overall synthesis, of catecholate siderophores contributes to virulence of extraintestinal pathogenic Escherichia coli. Mol Microbiol. 80:266-282; 2011.
18. Caza M, Lepine F, Milot S, Dozois C M. Specific roles of the iroBCDEN genes in virulence of an avian pathogenic Escherichia coli O78 strain and in production of salmochelins. Infect Immun. 76:3539-3549; 2008.
19. Dozois C M, Daigle F, Curtiss R 3rd. Identification of pathogen-specific and conserved genes expressed in vivo by an avian pathogenic Escherichia coli strain. Proc Natl Acad Sci USA. 100:247-252; 2003.
20. Emery D A, Nagaraja K V, Shaw D P, Newman J A, White D G. Virulence factors of Escherichia coli associated with colisepticemia in chickens and turkeys. Avian Dis. 36:504-511; 1992.
21. Garenaux A, Caza M, Dozois C M. The ins and outs of siderophore mediated iron uptake by extra-intestinal pathogenic Escherichia coli. Vet Microbiol. 153:89-98; 2011.
22. Gomis S M, Riddell C, Potter A A, Allan B J. Phenotypic and genotypic characterization of virulence factors of Escherichia coli isolated from broiler chickens with simultaneous occurrence of cellulitis and other colibacillosis lesions. Can J Vet Res. 65:1-6; 2001.
23. Sabri M, Caza M, Proulx J, Lymberopoulos M H, Bree A, Moulin-Schouleur M, Curtiss R, 3rd, Dozois C M. Contribution of the SitABCD, MntH, and FeoB metal transporters to the virulence of avian pathogenic Escherichia coli O78 strain chi7122. Infect Immun. 76:601-11; 2008.
24. Trampel D W, Wannemuehler Y, Nolan L K. Characterization of Escherichia coli isolates from peritonitis lesions in commercial laying hens. Avian Dis. 51:840-844; 2007.
25. Heidemann Olsen R, Bisgaard M, Christensen J P, Kabell S, Christensen H. Pathology and molecular characterization of Escherichia coli associated with the avian salpingitis-peritonitis disease syndrome. Avian Dis. 60:1-7; 2016.
26. Johnson T J, Siek K E, Johnson S J, Nolan L K. DNA sequence of a ColV plasmid and prevalence of selected plasmid-encoded virulence genes among avian Escherichia coli strains. J Bacteriol. 188:745-758; 2006.
27. Johnson T J, Skyberg J, Nolan L K. Multiple antimicrobial resistance region of a putative virulence plasmid from an Escherichia coli isolate incriminated in avian colibacillosis. Avian Dis. 48:351-360; 2004.
28. Johnson T J, Wannemuehler Y, Doetkott C, Johnson S J, Rose-nberger SC, Nolan L K. Identification of minimal predictors of avian pathogenic Escherichia coli virulence for use as a rapid diagnostic tool. J Clin Microbiol. 46:3987-3996; 2008.
29. Pires-Dos-Santos T, Bisgaard M, Christensen H. Genetic diversity and virulence profiles of Escherichia coli causing salpingitis and peritonitis in broiler breeders. Vet Microbiol. 162:873-880; 2013.
30. Skyberg J A, Johnson T J, Johnson J R, Clabots C, Logue C M, Nolan L K. Acquisition of avian pathogenic Escherichia coli μlasmids by a commensal E. coli isolate enhances its abilities to kill chicken embryos, grow in human urine, and colonize the murine kidney. Infect Immun. 74:6287-6292; 2006.
31. Tivendale K A, Allen J L, Browning G F. Plasmid-borne virulence-associated genes have a conserved organization in virulent strains of avian pathogenic Escherichia coli. J Clin Microbiol. 47:2513-2519; 2009.
32. Carver C, Griffith B, Huisinga A, Sandstrom J, Straub D. Vaccination with Siderophore receptors and porins protects against fowl cholera challenge by heterologous serotypes. In: Frame D D, editor. Proceedings of the 66th Western Poultry Disease Conference. 2017 March 20-22; Sacramento, CA. Jacksonville (FL): American Association of Avian Pathologists. p. 31-33; 2017.
33. Gorden P J, Kleinhenz M D, Ydstie J A, Brick T A, Slinden L M, Peterson M P, Straub D E, Burkhardt D T. Efficacy of vaccination with a Klebsiella pneumoniae siderophore receptor protein vaccine for reduction of Klebsiella mastitis in lactating cattle. J Dairy Sci. 101:10398-10408; 2018.
34. Bolin C A, Jensen A E. Passive immunization with antibodies against iron-regulated outer membrane proteins protects turkeys from Escherichia coli septicemia. Infect Immun. 55:1239-1242; 1987.
35. Holden K M, Browning G F, Noormohammadi A H, Markham P, Marenda M S. Avian pathogenic Escherichia coli DtonB mutants are safe and protective live-attenuated vaccine candidates. Vet Microbiol. 173:289-298; 2014.
36. Kariyawasam S, Wilkie B N, Gyles C L. Resistance of broiler chickens to Escherichia coli respiratory tract infection induced by passively transferred egg-yolk antibodies. Vet Microbiol. 98:273-284; 2004.
37. Cox G, Griffith B, Reed M, Sandstrom J, Petersen M, Straub D. Vaccination against Salmonella enterica Enteritidis using siderophore receptor and porin proteins. In: Frame D D, editor. Proceedings of the 66th Western Poultry Disease Conference. 2017 March 20-22; Sacramento, CA Jacksonville (FL): American Association of Avian Pathologists. p. 40-43; 2017.
38. Thomson D U, Loneragan G H, Thornton A B, Lechtenberg K F, Emery D A, Burkhardt D T, Nagaraja T G. Use of a siderophore receptor and porin proteins-based vaccine to control the burden of Escherichia coli O157:H7 in feedlot cattle. Foodborne Pathog Dis. 6:871-877; 2009.
39. Emery D, Straub, D, Slinden, L. Evaluation of a novel vaccine consisting of siderophore receptor proteins and porins for controlling salmonellosis in a commercial dairy herd. Proceedings of the 34th Annual Conference; 2001 September 13-15; Vancouver (Canada). Stillwater (OK): Frontier Printers, Inc. p. 132; 2001.
40. Hermesch D R, Thomson D U, Loneragan G H, Renter D R, White BJ. Effects of a commercially available vaccine against Salmonella enterica serotype Newport on milk production, somatic cell count, and shedding of Salmonella organisms in female dairy cattle with no clinical signs of salmonellosis. Am J Vet Res. 69:1229-1234; 2008.
41. National Chicken Council. Animal welfare guidelines. Washing-tonCity (DC): National Chicken Council, September, 2020. https://www.nationalchickencouncil.org/wp-content/uploads/2021/02/NCC-Animal-Welfare-Guidelines_Broilers_Sept2020.pdfPublisher; year.
42. Animal and Plant Health Inspection Service, United States Department of Agriculture. 9 CFR 108.10. Outer premises and stables. Washington, DC.
43. Animal and Plant Health Inspection Service, United States Department of Agriculture. 9 CFR 117.1-117.6. Animals at licensed establishments. Washington, DC.
44. GraphPad Software, Inc. Prism, version 6.07 La Jolla (CA): GraphPad Software, Inc. https://www.graphpad.com/scientific-software/prism/URL; 2015.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-4. (canceled)

5. A method for treating peritonitis or a sign of peritonitis in a subject, the method comprising:
administering an effective amount of the composition to a subject having or at risk of having peritonitis caused by E. coli, wherein the subject is a domesticated fowl, wherein the composition comprises any two, three, four, five, six, seven, or eight of the proteins chosen from:
an isolated protein that has at least 80% identity with SEQ ID NO:2,
an isolated protein that has at least 80% identity with SEQ ID NO:4,
an isolated protein that has at least 80% identity with SEQ ID NO:6,
an isolated protein that has at least 80% identity with SEQ ID NO:8,
an isolated protein that has at least 80% identity with SEQ ID NO:10,
an isolated protein that has at least 80% identity with SEQ ID NO:12,
an isolated protein that has at least 80% identity with SEQ ID NO:20,
an isolated protein that has at least 80% identity with SEQ ID NO:22, and
an isolated protein that has at least 80% identity with SEQ ID NO:44,
the composition further comprising a pharmaceutically acceptable carrier and
an adjuvant.

6-10. (canceled)

11. A method for treating peritonitis or a sign of peritonitis in a subject, the method comprising:
administering an effective amount of a composition to a subject having or at risk of having peritonitis caused by E. coli, wherein the composition comprises antibody that specifically binds to a protein of a composition, wherein the subject is a domesticated fowl, and wherein the composition comprises any two, three, four, five, six, seven, or eight of the proteins chosen from:
an isolated protein that has at least 80% identity with SEQ ID NO:2,
an isolated protein that has at least 80% identity with SEQ ID NO:4,
an isolated protein that has at least 80% identity with SEQ ID NO:6,
an isolated protein that has at least 80% identity with SEQ ID NO:8,
an isolated protein that has at least 80% identity with SEQ ID NO:10,
an isolated protein that has at least 80% identity with SEQ ID NO:12,
an isolated protein that has at least 80% identity with SEQ ID NO:20,
an isolated protein that has at least 80% identity with SEQ ID NO:22, and
an isolated protein that has at least 80% identity with SEQ ID NO:44,
the composition further comprising a pharmaceutically acceptable carrier and
an adjuvant.

12-15. (canceled)

16. The method of claim 5, wherein the domesticated fowl is a chicken, a turkey, or a duck.

17. The method of claim 5, wherein at least 0.01 micrograms (μg) and no greater than 500 μg of protein is administered.

18-21. (canceled)

22. The method of claim 5, wherein the composition comprises:
an isolated protein that has at least 80% identity with SEQ ID NO:2,
an isolated protein that has at least 80% identity with SEQ ID NO:4,
an isolated protein that has at least 80% identity with SEQ ID NO:6,
an isolated protein that has at least 80% identity with SEQ ID NO:8,
an isolated protein that has at least 80% identity with SEQ ID NO:10,
an isolated protein that has at least 80% identity with SEQ ID NO:12,
the pharmaceutically acceptable carrier, and
the adjuvant.

23. The method of claim 22, wherein the composition further comprises:
an isolated protein that has at least 80% identity with SEQ ID NO:20,
an isolated protein that has at least 80% identity with SEQ ID NO:22, or
an isolated protein that has at least 80% identity with SEQ ID NO:20 and an isolated
protein that has at least 80% identity with SEQ ID NO:22.

24. The method of claim 5, wherein the composition comprises:
an isolated protein that has at least 80% identity with SEQ ID NO:4, an isolated protein that has at least 80% identity with SEQ ID NO:2, an isolated protein that has at least 80% identity with SEQ ID NO:8, and an isolated protein that has at least 80% identity with SEQ ID NO:6;
an isolated protein that has at least 80% identity with SEQ ID NO:2, an isolated protein that has at least 80% identity with SEQ ID NO:6, and an isolated protein that has at least 80% identity with SEQ ID NO:44;
an isolated protein that has at least 80% identity with SEQ ID NO:4 and an isolated protein that has at least 80% identity with SEQ ID NO:2;
an isolated protein that has at least 80% identity with SEQ ID NO:8 and an isolated protein that has at least 80% identity with SEQ ID NO:6; or
an isolated protein that has at least 80% identity with SEQ ID NO:2 and an isolated protein that has at least 80% identity with SEQ ID NO:6;
the pharmaceutically acceptable carrier and
the adjuvant.

25. The method of claim 11, wherein the composition comprises:
an isolated protein that has at least 80% identity with SEQ ID NO:2,
an isolated protein that has at least 80% identity with SEQ ID NO:4,
an isolated protein that has at least 80% identity with SEQ ID NO:6,
an isolated protein that has at least 80% identity with SEQ ID NO:8,
an isolated protein that has at least 80% identity with SEQ ID NO:10,
an isolated protein that has at least 80% identity with SEQ ID NO:12,
the pharmaceutically acceptable carrier, and
the adjuvant.

26. The method of claim 25, wherein the composition further comprises:
an isolated protein that has at least 80% identity with SEQ ID NO:20,
an isolated protein that has at least 80% identity with SEQ ID NO:22, or
an isolated protein that has at least 80% identity with SEQ ID NO:20 and an isolated protein that has at least 80% identity with SEQ ID NO:22.

27. The method of claim 11, wherein the composition comprises:
an isolated protein that has at least 80% identity with SEQ ID NO:4, an isolated protein that has at least 80% identity with SEQ ID NO:2, an isolated protein that has at least 80% identity with SEQ ID NO:8, and an isolated protein that has at least 80% identity with SEQ ID NO:6;
an isolated protein that has at least 80% identity with SEQ ID NO:2, an isolated protein that has at least 80% identity with SEQ ID NO:6, and an isolated protein that has at least 80% identity with SEQ ID NO:44;
an isolated protein that has at least 80% identity with SEQ ID NO:4 and an isolated protein that has at least 80% identity with SEQ ID NO:2;
an isolated protein that has at least 80% identity with SEQ ID NO:8 and an isolated protein that has at least 80% identity with SEQ ID NO:6; or
an isolated protein that has at least 80% identity with SEQ ID NO:2 and an isolated protein that has at least 80% identity with SEQ ID NO:6;
the pharmaceutically acceptable carrier and
the adjuvant.

28. The method of claim 11, wherein the domesticated fowl is a chicken, a turkey, or a duck.

29. The method of claim 11, wherein at least 0.01 micrograms (μg) and no greater than 500 μg of protein is administered.
